(19) United States
(12) Patent Application Publication
(10) Pub. No.: US 2026/0317760 A1
(43) Pub. Date: Oct. 15, 2026

(54) TARGETING LONG NON-CODING RNA CHROMR IN INTERFERON-MEDIATED INFLAMMATION IN HUMANS

(71) Applicant: New York University, New York, NY (US)

(72) Inventors: Kathryn J. MOORE, Westfield, NJ (US); Coen VAN SOLINGEN, New York, NY (US); Yannick CYR, New York, NY (US)

(21) Appl. No.: 18/996,924

(22) PCT Filed: Jul. 27, 2023
(86) PCT No.: PCT/US2023/071135

§ 371 Date: Jan. 17, 2025

Related U.S. Application Data
(60) Provisional application No. 63/393,228, filed on Jul. 28, 2022.

(51) Int. Cl. C12N 15/113; A61P 29/00

(52) U.S. Cl. CPC C12N 15/113; A61P 29/00; C12N 2310/113

(57) ABSTRACT

Provided are compositions and methods for prophylaxis or treatment of a disorder associated with type I interferon induced inflammation. The compositions involve use of an agent that inhibits the function and/or reduces the level of long non-coding RNA Cholesterol Homeostasis Regulator of Micro-RNA expression RNA (lncRNA CHROMR) in the individual to thereby reduce the severity of the type I interferon induced inflammation. The agents are provided as antisense oligonucleotides.

### CROSS-REFERENCE TO RELATED APPLICATIONS

This application claims priority to U.S. provisional application Ser. No. 63/393,228, filed Jul. 28, 2022, the entire disclosure of which is incorporated herein by reference.

### STATEMENT REGARDING FEDERALLY SPONSORED RESEARCH

This invention was made with government support under grant nos. R35HL13579 and P01HL131481 awarded by the National Institutes of Health. The government has certain rights in the invention.

### SEQUENCE LISTING

The instant application contains a Sequence Listing which is submitted in .xml format and is hereby incorporated by reference in its entirety. Said .xml file is named “TARGETING LONG NON-CODING RNA CHROMR.xml”, was created on Jul. 25, 2023, and is 136,234 bytes in size.

### FIELD

The present disclosure relates generally to approaches for treatment of conditions that are associated with type I interferon induced inflammation by targeting RNA Cholesterol Homeostasis Regulator of Micro-RNA expression RNA (lncRNA CHROMR).

### BACKGROUND

Human respiratory viruses, including influenza and severe acute respiratory syndrome coronavirus 2 (SARS-CoV-2), are major causes of morbidity and mortality worldwide. Effective antiviral immunity relies on the activation of conserved innate immune signaling pathways that coordinate the production of type I interferons (IFNα/β) and the expression of several hundred interferon-stimulated genes (ISGs), which collectively subvert viral entry, replication and pathogenesis⁽¹⁾. IFNα/β are secreted cytokines that bind IFNα/β receptors (IFNARs) to initiate JAK-STAT signaling and the assembly of the IFN-stimulated gene factor 3 complex (ISGF3), consisting of interferon regulatory factor (IRF)-9 together with a STAT1-STAT2 heterodimer. This complex transcriptionally activates target genes harboring regulatory IFN-stimulated response elements (ISRE), culminating in the expression of hundreds of ISGs^{(1, 2)}. In addition, constitutive and IFN-induced ISG expression can also be regulated by IRF-1 binding of ISREs^{(2, 3)}. These pathways must be strictly controlled, as dysregulation of IFN production, signaling or ISG expression can lead to persistent inflammation and autoimmune disorders, such as systemic lupus erythematosus and Aicardi-Goutieres syndrome⁽⁴⁾.

Emerging evidence suggests that long non-coding RNAs (lncRNAs) regulate the expression of protein coding genes and their interaction networks in diverse biological processes, including innate immunity^{(5, 6)}. Defined as RNA transcripts longer than 200 nucleotides that lack protein-coding potential, lncRNAs execute their structural and regulatory functions by interacting with DNA, protein or other RNAs in the nucleus or cytoplasm. LncRNAs contribute to gene regulation through diverse mechanisms, including through guiding or sequestering chromatin-modifying enzymes and transcriptional complexes in the nucleus; regulating mRNA processing, splicing and translation; and acting as competitive inhibitors of endogenous RNAs (e.g., microRNAs) or proteins in the cytoplasm^{(7, 8)}. To date, a limited number of lncRNAs have been described to regulate the IFN response by altering the function of viral sensors, production of IFNs, and expression of ISGs. For example, lncATV⁽⁹⁾ and lncRNA-LSm3b⁽¹⁰⁾ have been shown to interact with the cytosolic double stranded (ds) RNA sensor RIG-I and restrict its function, whereas Lnczc3h7a promotes RIG-I function by enabling its interaction with TRIM25⁽¹¹⁾. Similarly, Inc-ITPRIP-1 binds and enhances the function of the RIG-I-like receptor MDA5 (IFIH1)⁽¹²⁾. Other lncRNAs have been shown to be induced by IFN-I and mediate feedback inhibition of IFN responses, such as Inc-MxA, which negatively regulates IFNβ expression by impeding NF-κB and IRF3 binding at its promoter⁽¹³), and LUCAT1, which binds and sequesters STAT1 in the nucleus to limit IFN signaling⁽¹⁴⁾. BISPR is an example of a lncRNA expressed from a bidirectional promoter that cis-regulates expression of its neighboring gene, BST2 (Tetherin), an ISG that is known to prevent infection⁽¹⁵⁾. CCR5AS behaves as a decoy for the RNA-binding protein RALY, preventing its binding to and repression of the chemokine receptor CCR5⁽¹⁶⁾. Finally, lncRNA-CMPK2⁽¹⁷⁾, NRAV⁽¹⁸⁾, and NRIR⁽¹⁹⁾ have been shown to broadly alter ISG expression, although the exact mechanisms remain unclear.

Most lncRNAs exhibit poor evolutionary conservation suggesting that functional investigation of human lncRNAs that modulate the IFN response and antiviral immunity may unveil key points of pathogen control and novel targets for therapeutic intervention. There is accordingly an unmet need for identification of targets that can be modulated to address aberrant expression of ISREs and dysregulation of IFN production, signaling, or ISG expression for use in prophylaxis and therapy of conditions associated with persistent inflammation such as autoimmune and other disorders. The present disclosure is pertinent to this need.

### BRIEF SUMMARY

In this disclosure it is shown that CHROMR, a primate-specific lncRNA first identified to regulate cellular lipid metabolism⁽²⁰⁾, is highly induced in the patients infected with the influenza virus or SARS-CoV-2, and in human primary macrophages and cell lines exposed to RNA viruses or the synthetic dsRNA polyinosinic:cytidylic acid (poly(I:C)). Loss-of-function studies identify an important role for CHROMR in the regulation of ISG expression, and restriction of influenza virus replication in macrophages. While activation of NF-κB signaling is intact in CHROMR-depleted macrophages, these cells exhibit reduced expression of an IRF-inducible ISRE luciferase reporter gene indicating a defect in transcriptional activation of IRF signaling and interferon response pathways. The disclosure also reveals that CHROMR sequesters the nuclear transcriptional co-repressor IRF2BP2, which acts together with IRF-2 to repress ISG transcription, thereby licensing IRF-dependent signaling and transcription of the ISG network. These results provide insights into the multilayered regulatory network that controls ISG expression and the innate immune response to viruses and that targeting CHROMR is an approach to treating conditions associated with type I interferon induced inflammation. In this regard, the disclosure also shows that CHROMR expression correlates with systemic lupus erythematosus (SLE) two-score IFN system signature, and knocking down CHROMR expression using antisense oligonucleotides leads to reduced IRF transcriptional activity induced by SLE-relevant agonists Imiquimod. The disclosure also demonstrates that a CHROMR-targeting 7-Mer (ASO_7/1) designed against the first GG-pair of a G-quadruplex present in CHROMR efficiently decreases IFNβ-induced ISG expression without causing CHROMR degradation. The disclosure also demonstrates that a CHROMR-targeting 13-Mer (ASO_13/1) antisense oligonucleotide designed against the first two GG-pairs of a G-quadruplex present in CHROMR efficiently decrease IFNβ-induced ISG expression without causing CHROMR degradation. The disclosure also demonstrates that CHROMR-targeting GapmeR (ASO_Gap/3) designed to cause CHROMR degradation efficiently decreases IFNβ-induced ISG expression and decreases CHROMR expression. The disclosure also demonstrates that CHROMR-targeting antisense oligonucleotides are efficient at reducing cytokine secretion in human vascular explants.

### BRIEF DESCRIPTION OF THE FIGURES

FIG. 1. LncRNA CHROMR is upregulated in SARS-CoV-2 and influenza A infected patients and correlates with transcriptional activation of antiviral gene programs. (A) Experimental design for identification of lncRNAs differentially expressed in whole blood of patients with influenza A virus or SARS-CoV-2 and controls. (B) Scatter plot of the lncRNAs identified as commonly dysregulated in SARS-CoV-2 and influenza A virus infected patients by whole blood high-throughput RNA-sequencing. Upregulated lncRNAs are indicated in red (n=116) and downregulated in blue (n=75); −1.5<fold change (FC)>1.5; P-adj<0.05. Non-significantly changed lncRNAs are indicated in grey. (C) Normalized transcript expression (CPM) of CHROMR and lncRNAs described to regulate interferon responses in blood of control subjects [n=18 (IAV), n=7 (SARS-CoV-2)], and patients infected with influenza A virus (IAV, n=41) or SARS-CoV-2 (CoV-2, n=8). (D) Violin plot showing the distributions of the Pearson correlation coefficient between indicated lncRNAs and 226 differentially expressed interferon-stimulated genes (ISGs) common to IAV- and CoV-2-infected patients. (E) Robust third-order non-linear fit of the lncRNA×ISG Pearson correlation coefficient displayed as a function of the differential expression of the ISGs. (F) Pearson correlation matrix showing the 30 ISGs that are most strongly associated with CHROMR in whole blood in IAV-infected patients. (G) Scatter plot of the Pearson coefficients of bivariate correlations between the 226 ISGs and the corresponding CHROMR-corrected partial correlation coefficients with correlations that are significantly changed by correcting for CHROMR expression highlighted in green. Blue dots representing functionally related gene-gene pairs are displayed in (H). (H) CHROMR-associated ISG interactome clustered on the basis of functional relationships. Data are mean±standard error of the mean (C)±quartiles (D), third order polynomial non-linear fit with robust adjustment (E). P values were calculated using a one-way ANOVA, with Sidak's multiple comparison test (C), or Kruskal-Wallis test with Dunn's correction for multiple comparison (D). All bivariate and partial correlation analyses performed in influenza A infected patients (n=41) (D to H). All data log₁ₚ transformed for linear regression analysis (F to H). Difference in correlation coefficient assessed by Fisher r-to-Z transformation followed by Z-test (G). *P≤0.05; **P<0.01; ***P<0.001; ****P<0.0001.

FIG. 2. CHROMR deficiency leads to diminished expression of interferon-stimulated genes. (A) Time course of CHROMR expression (FPKM) in human monocyte-derived macrophages infected with influenza A/California/04/09 (H1N1), influenza A/Wyoming/03/03 (H3N2), or mock infected. (B) qPCR analysis of CHROMR in human THP-1 macrophages infected with influenza A virus/WSN/1933 (H1N1, 1000 PFU) or stimulated with the synthetic dsRNA poly(I:C) (1 μg/mL). (C) Volcano plot showing differentially expressed genes in CHROMR-depleted (GapCHROMR-treated) and control (GapCTRL-treated) THP-1 macrophages after poly(I:C) (1 μg/mL, 8 h) stimulation and RNA-seq. Dashed lines indicate fold change (log₂)=±1; P-adj=0.05; red dots indicate upregulated genes; blue dots indicate downregulated genes; grey dots indicate non-significantly changed genes. (D) Hierarchical clustering heatmap showing normalized gene expression values in THP-1 macrophages treated with GapCHROMR or GapCTRL in poly(I:C) stimulated conditions (1 μg/mL, 8 h). Cutoffs used for visualization: −2<fold change (FC)>2; and P-adj<0.05. (E) List of most affected canonical pathways identified through Ingenuity Pathway Analysis of (C) ranked by P-adj. (F) Expression of top chemokine genes differentially regulated in CHROMR-depleted and control THP-1 macrophages. Top row: RNA-seq normalized expression counts (CPM) after poly(I:C) (1 μg/mL, 8 h), bottom row: immunoassay of protein levels after poly(I:C) (1 μg/mL, 24 h). (G) Gene expression profiling of 84 interferon stimulated genes in THP-1 macrophages stably overexpressing CHROMR or an empty vector control upregulated genes are indicated in red and downregulated genes in blue. Genes indicated are P<0.1). (H and I) Predicted cytokine (H) and transcriptional regulators (I) of differentially expressed genes in (C); dashed lines indicate Z-score=±2 and P-adj=0.05; red and blue dots indicate significantly upregulated and downregulated factors, respectively. Data related to a constructed hierarchical clustering heatmap based on Z-scores of differentially expressed interferon-stimulated genes in CHROMR-depleted and control THP-1 macrophages treated with poly(I:C) (1 μg/mL) for 8 h, P-adj<0.05. Data are mean±standard error of the mean for 2 (A), 3 (B to F (top), G) independent experiments, or representative of 3 independent experiments (F (bottom)). P values were calculated using a repeated measures two-way ANOVA with Sidak's multiple comparison test (A), one-way ANOVA with Dunnett's multiple comparison test (B), right-tailed Fisher's exact test (E, H and I), or a two-tailed unpaired Student's t-test (F and G). ^{#}P<0.1; *P<0.05; **P<0.01; ***P<0.001; ****P<0.0001. The data related to the hierarchical clustering for differentially expressed interferon-stimulated genes in CHROMR-depleted and control THP-1 macrophages treated with poly(I:C) are shown in Extended Data Table 3.

FIG. 3. CHROMR is required to restrict influenza virus and activate interferon stimulated gene transcription. (A) Percentage of viral infection in CHROMR-depleted (GapCHROMR-treated) and control (GapCTRL-treated) THP-1 macrophages challenged with influenza A virus/WSN/1933 (H1N1) at increasing plaque forming units (PFU). Percentages were calculated relative to GapCTRL transfection at highest infection rate. (B) Transcription factor binding enrichment scores for interferon stimulated genes (ISG) differentially expressed in CHROMR-depleted and control THP-1 macrophages stimulated with poly(I:C) using the ChIP Enrichment Analysis (ChEA 2016) database gene set library. (C) Reporter assay for IRF-driven transcription (Top: luciferase, Relative units, RU) or NF-κB-driven transcription (bottom: secreted alkaline phosphatase, SEAP) in THP-1 Dual Reporter macrophages transfected with GapCHROMR or GapCTRL and left untreated or stimulated with poly(I:C) (1 μg/mL). Relative expression is normalized to time 0 (=100). (D) Volcano plot showing differential H3K27Ac modification in CHROMR-depleted and control THP-1 macrophages stimulated with poly(I:C). ChIP-seq reads that are gained or lost after CHROMR knockdown are indicated in red and blue, respectively. Dashed line indicates P-adj<0.1. (E) Genomic distribution of H3K27Ac marks lost after CHROMR knockdown identified in D, P-adj<0.1. (F) List of biological processes identified using the Genomic Regions Enrichment Annotations Tool (GREAT) analysis of H3K27Ac-depleted promoter regions. (G) Metagene plots showing the mean (top) and individual unique positions (bottom) of normalized H3K27Ac read density around the transcription start site (TSS±1500 base pairs) of ISGs in THP-1 macrophages transfected with GapCHROMR or GapCTRL. (H) Hypergeometric Optimization of Motif EnRichment (HOMER) analysis of promoter regions depleted of H3K27Ac after CHROMR knockdown showing transcription factors with highest similarity score in motif indicated in bars. Data are mean±standard error of the mean for 3 independent experiments. P values were calculated using a repeated measures two-way ANOVA with Sidak's multiple comparison test (A and C) or Binomial test (B, F and H). *P≤0.05; **P<0.01; ***P<0.001.

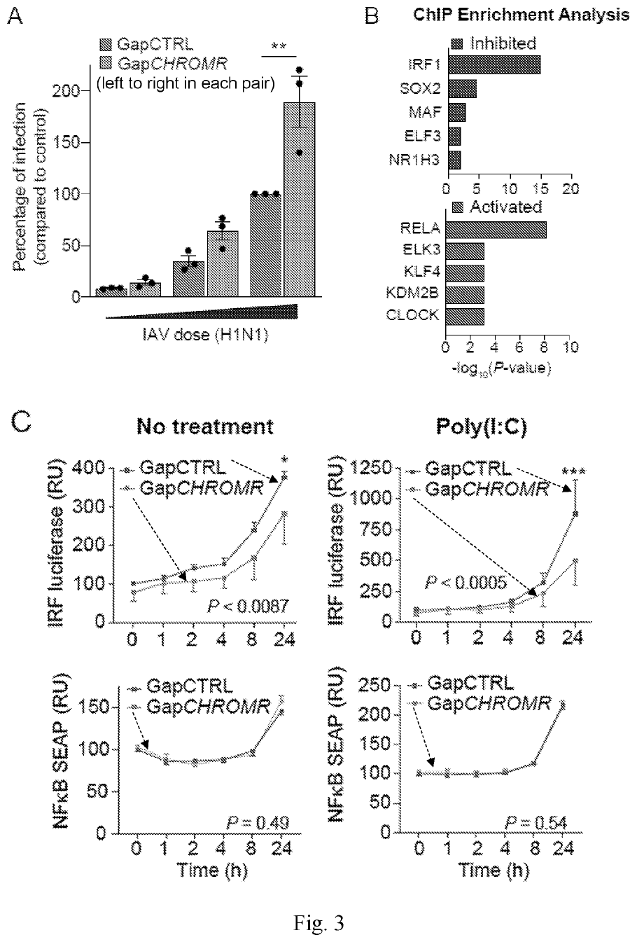

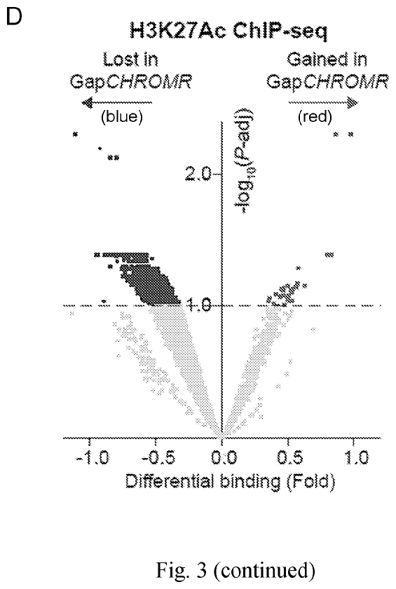

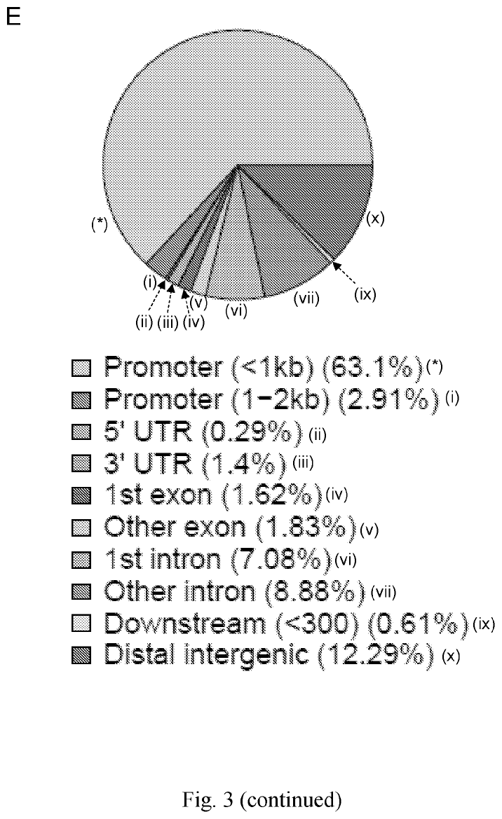

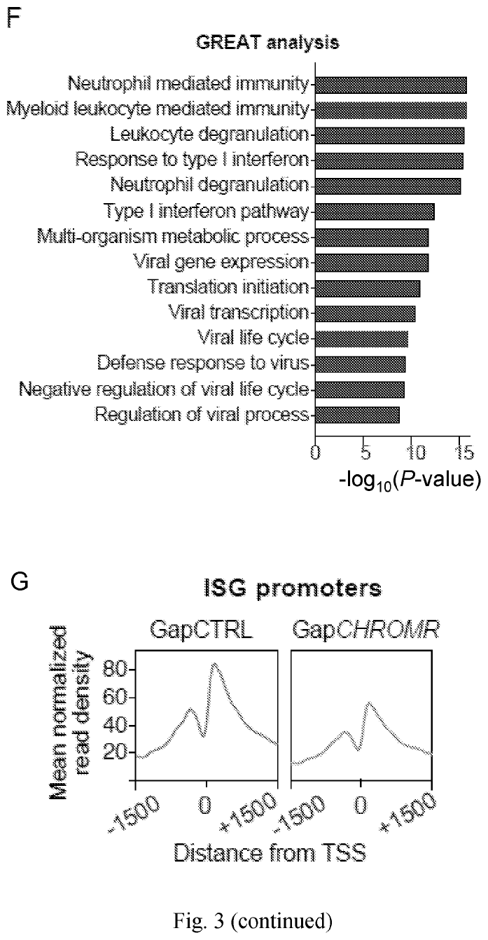

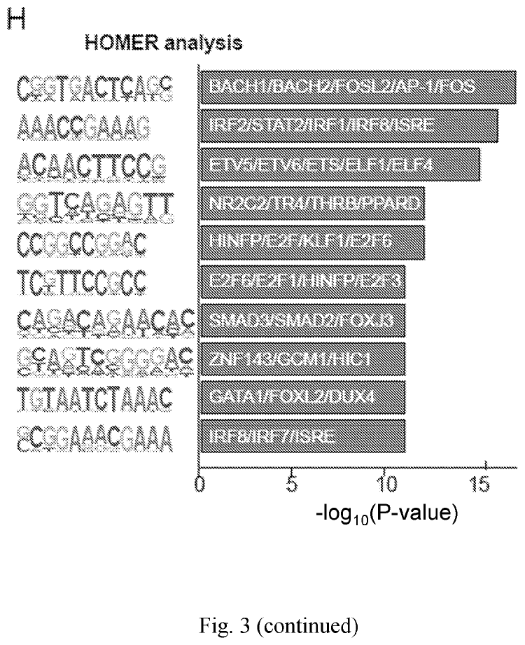

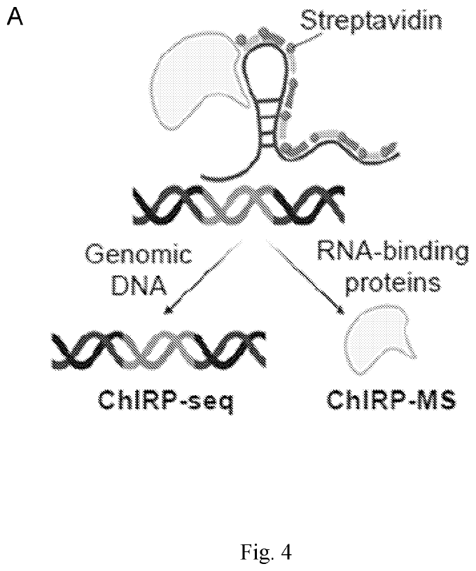

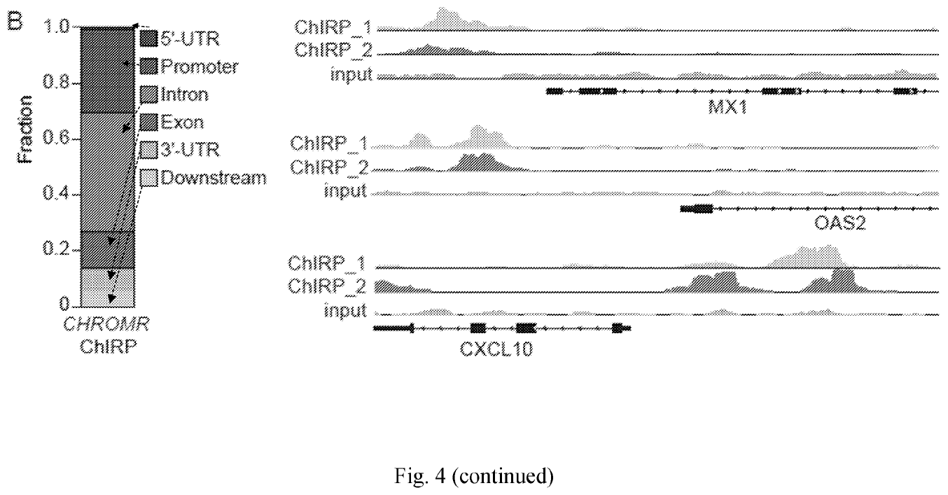

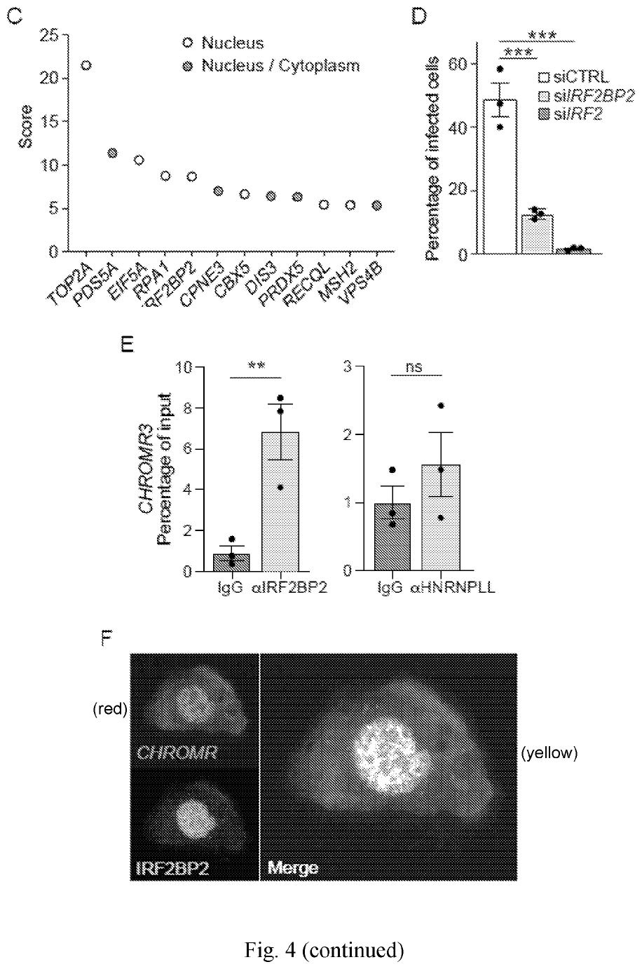

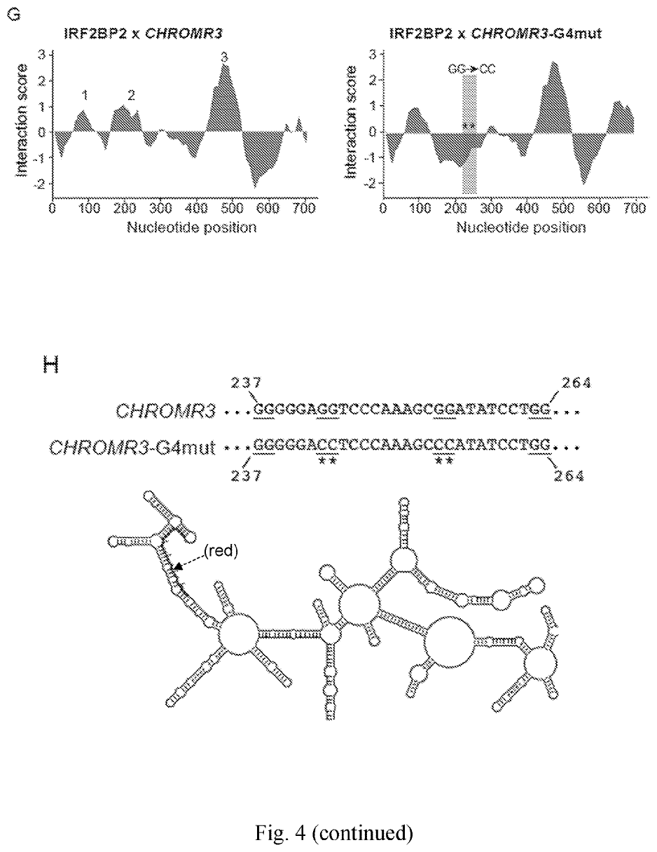

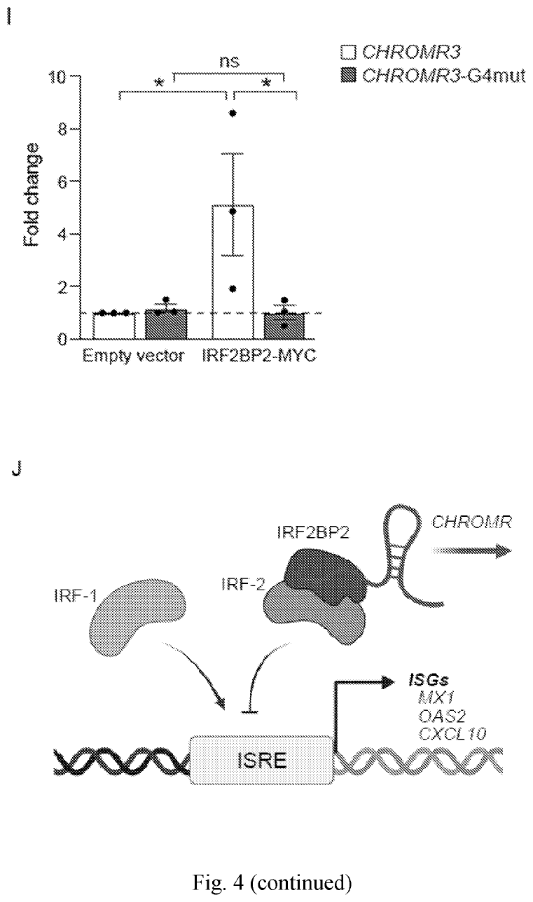

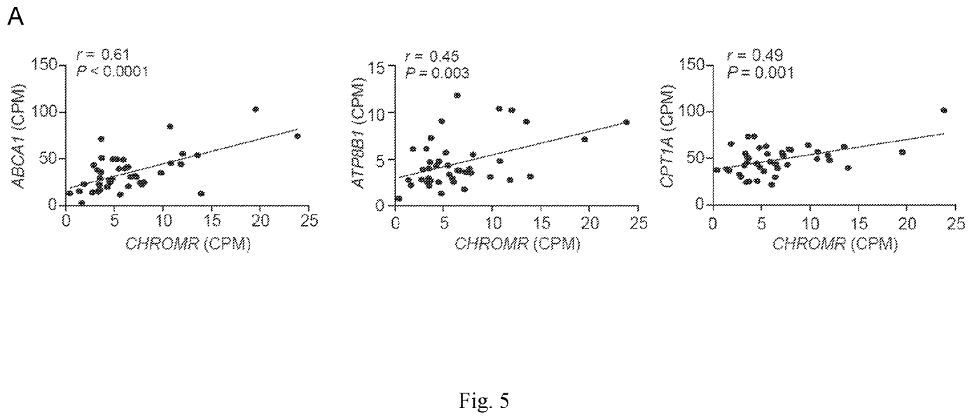

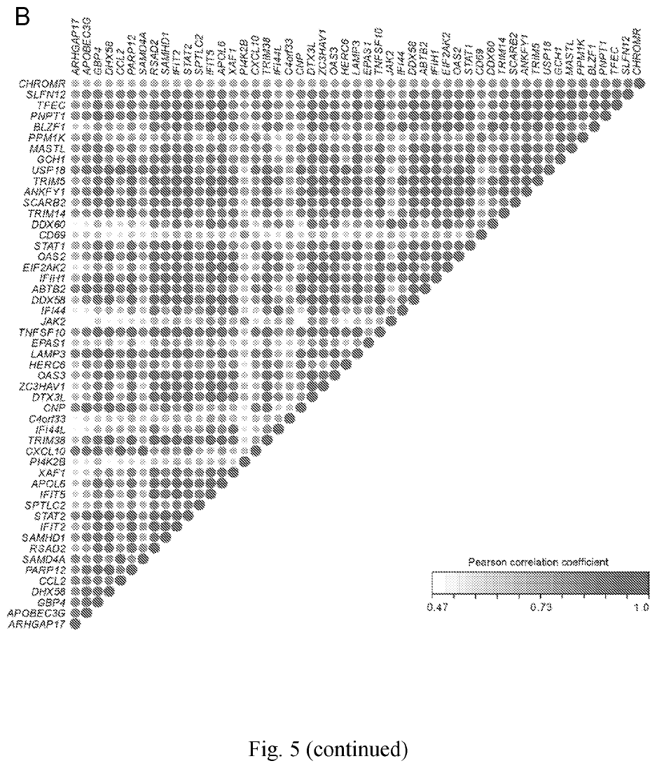

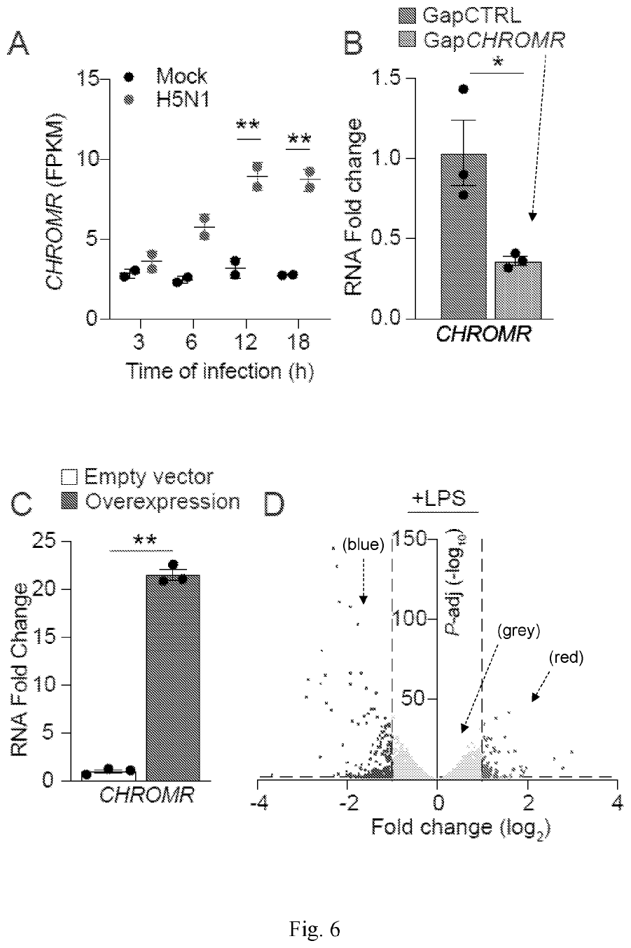

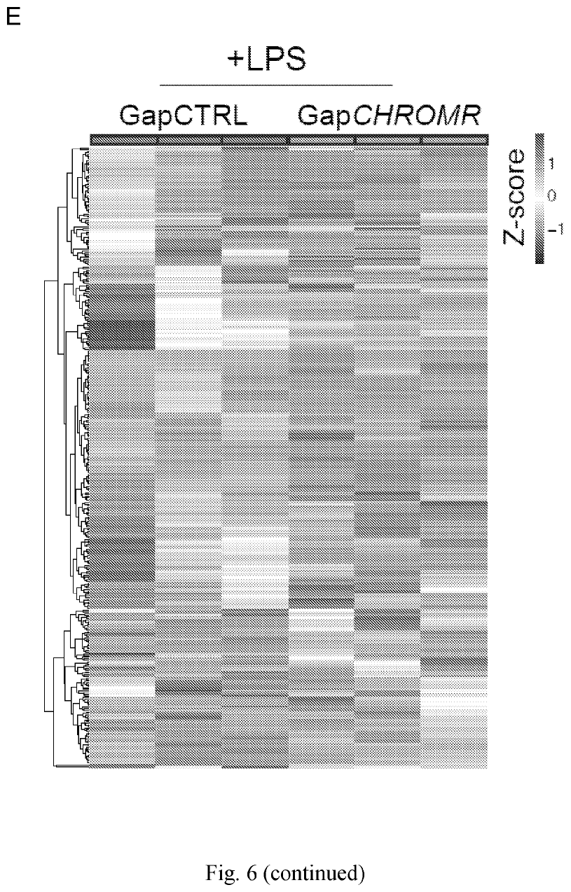

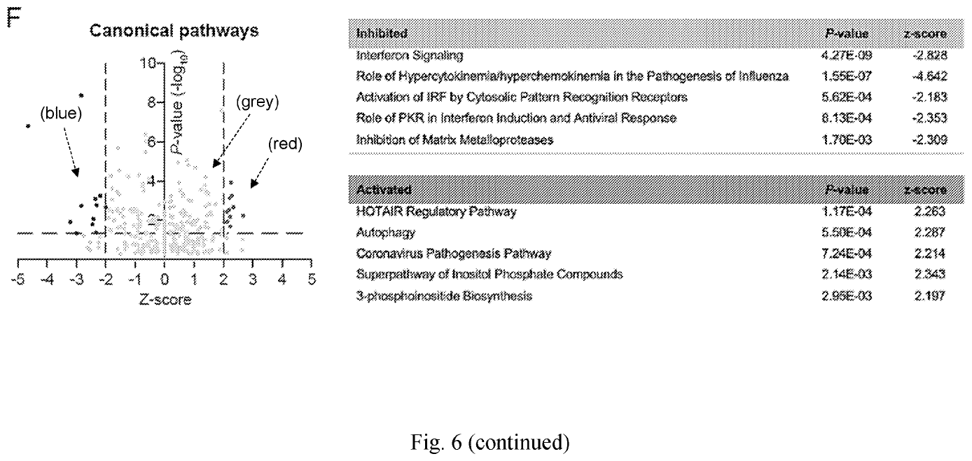

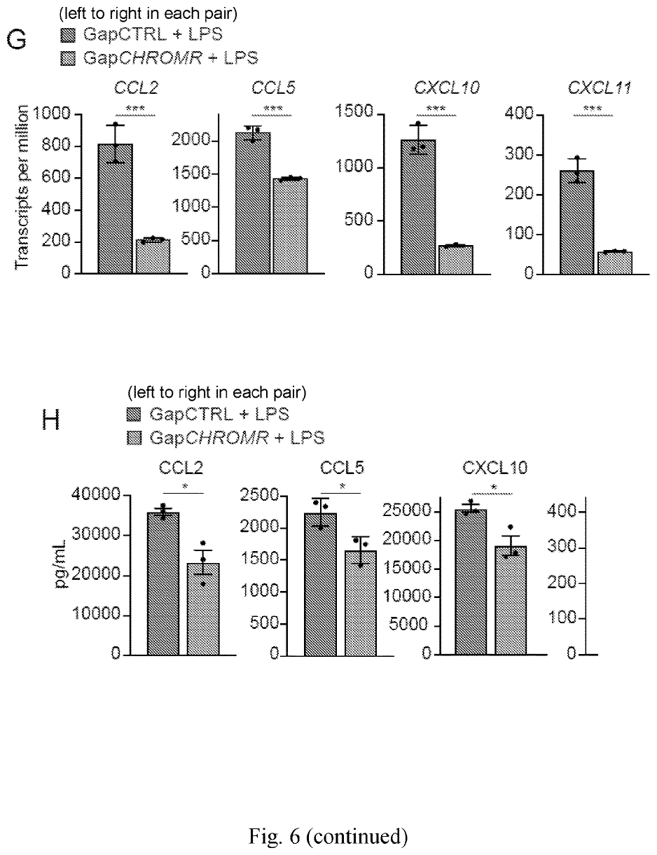

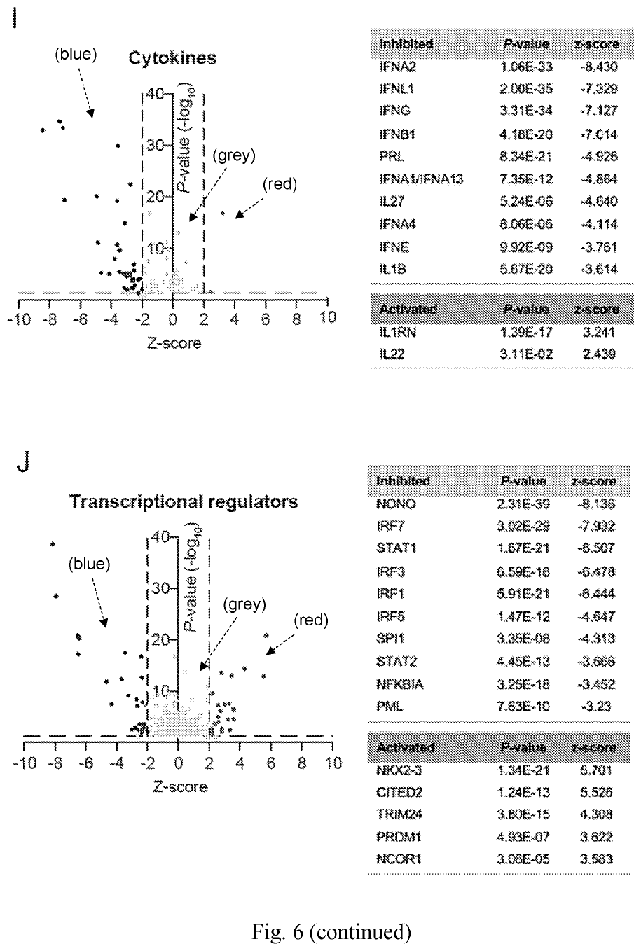

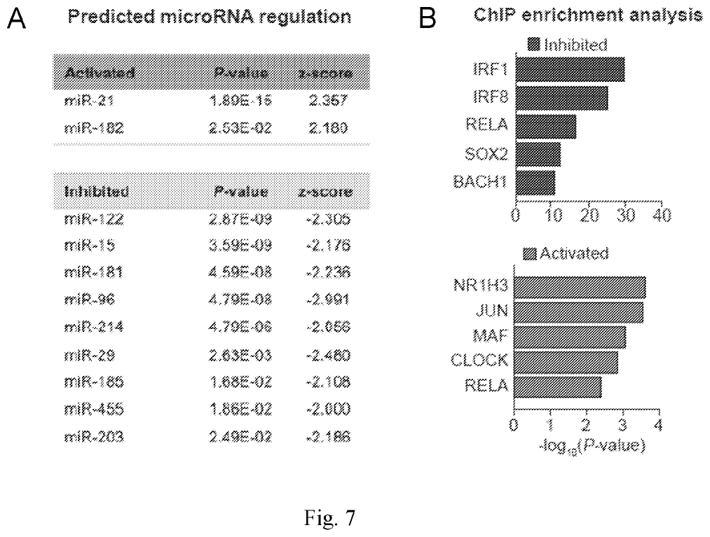

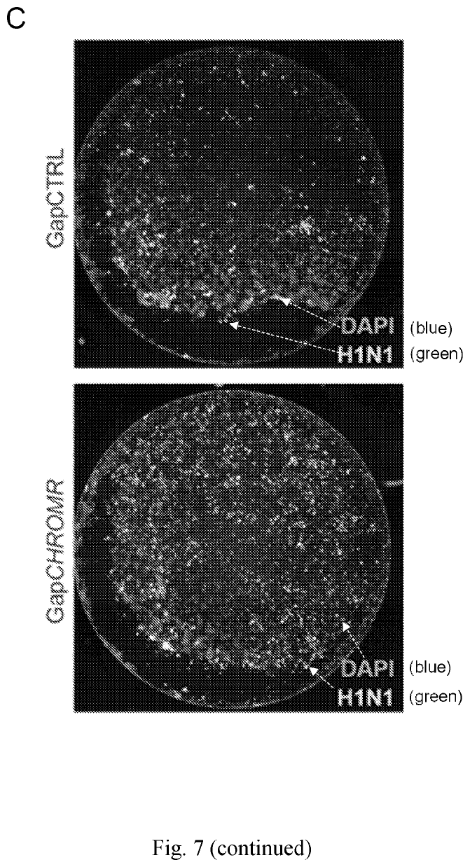

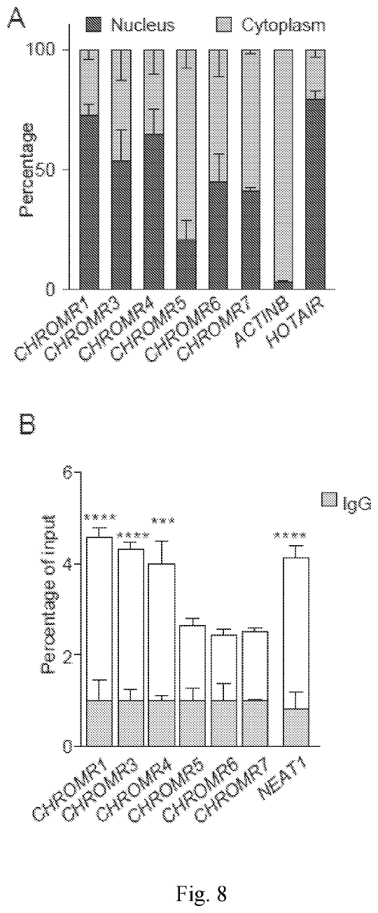

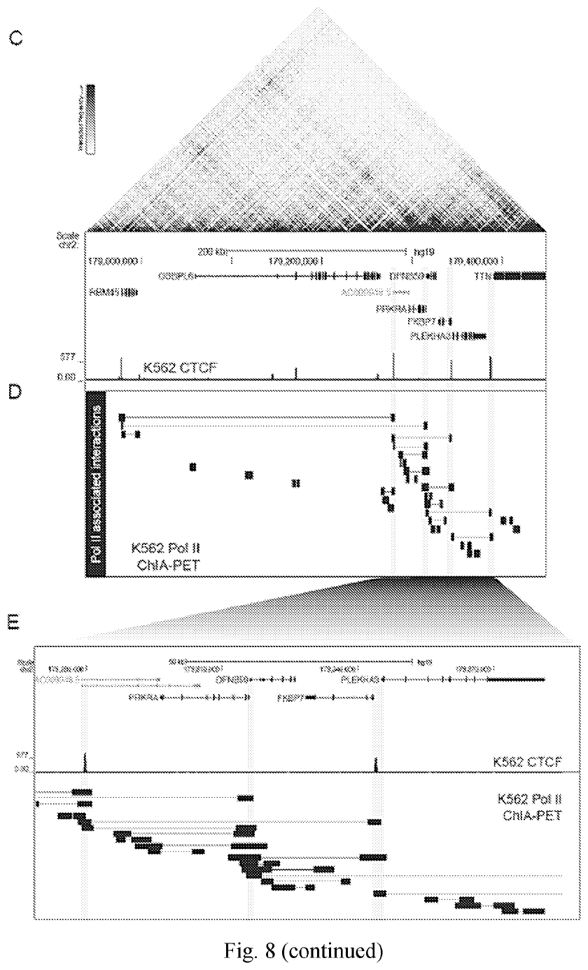

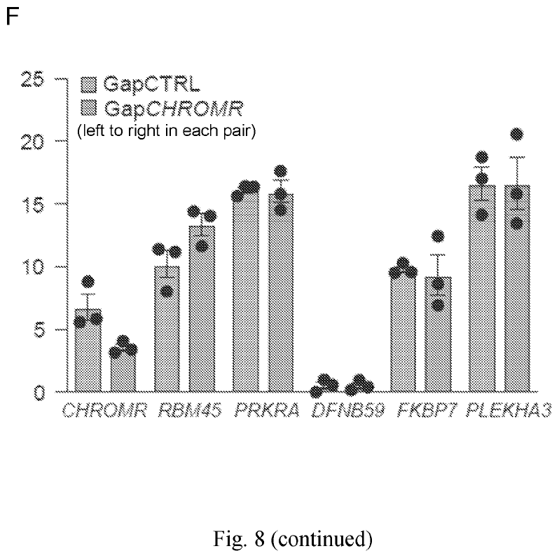

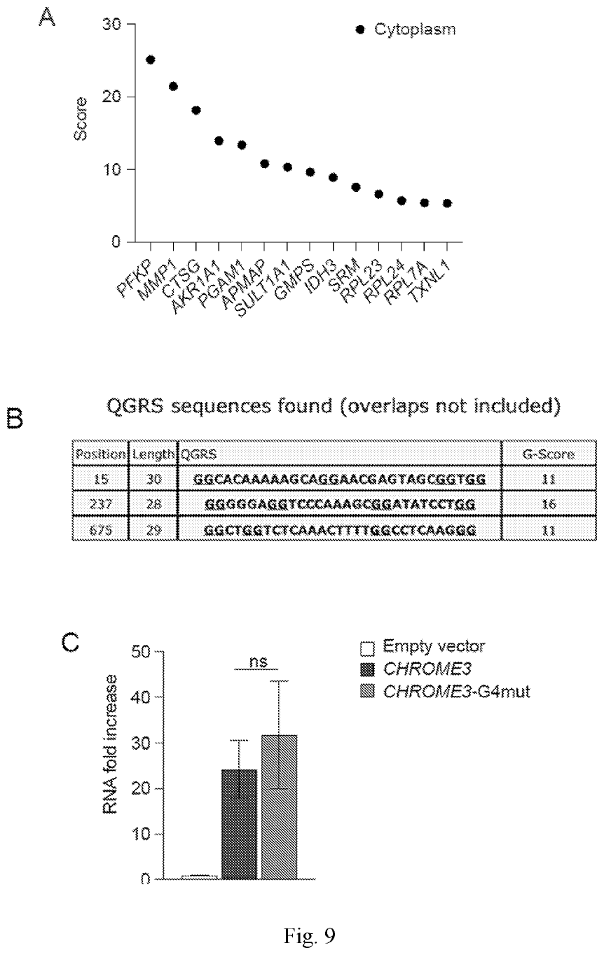

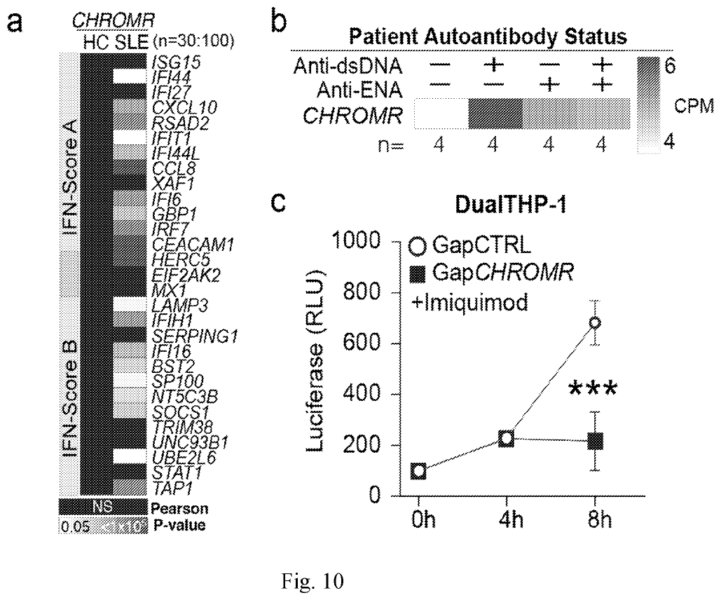

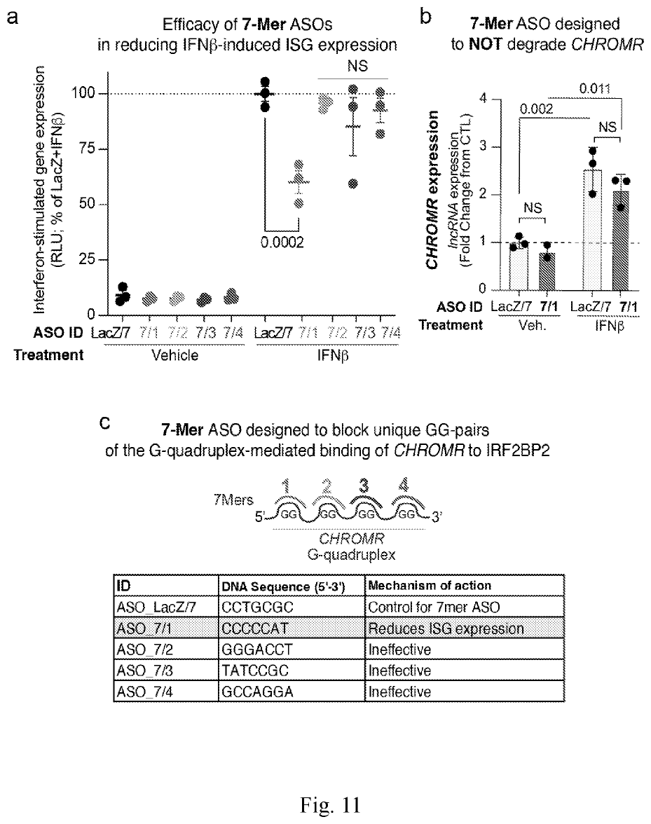

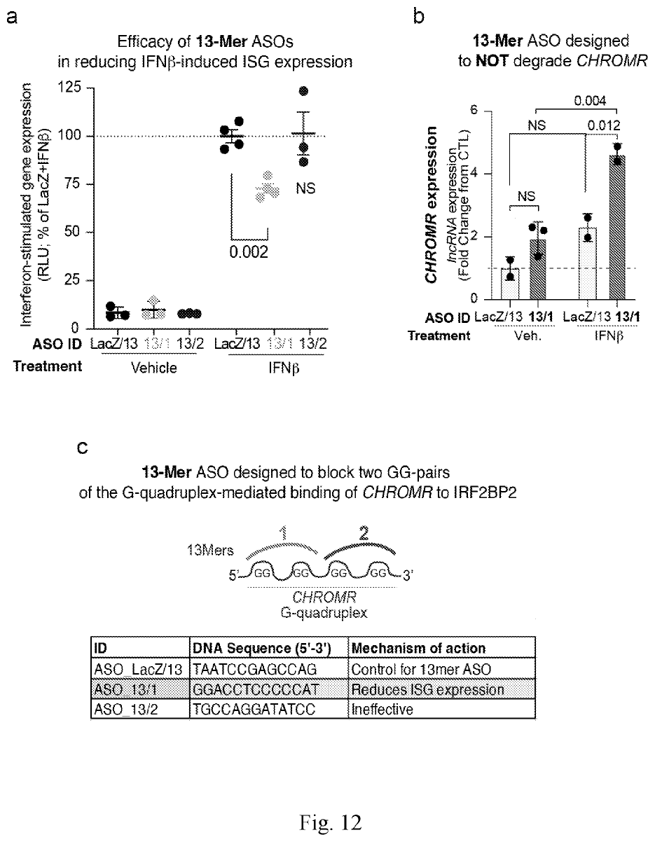

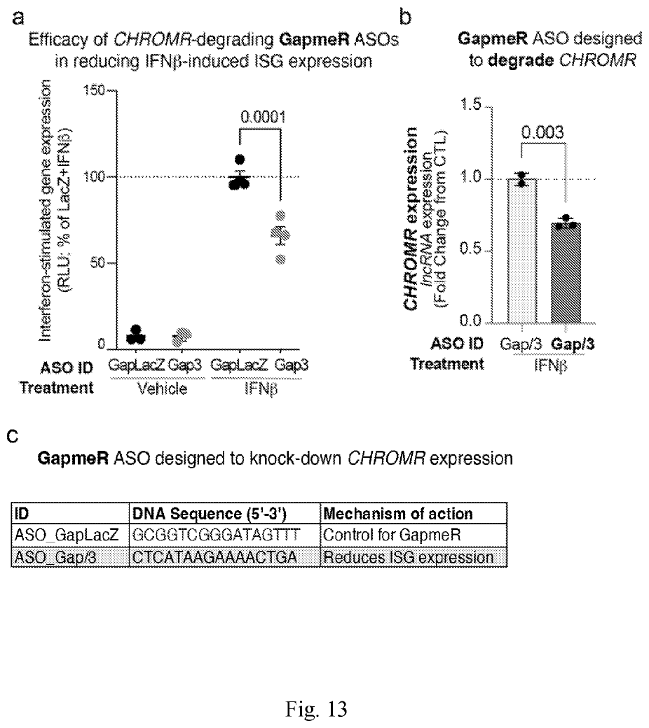

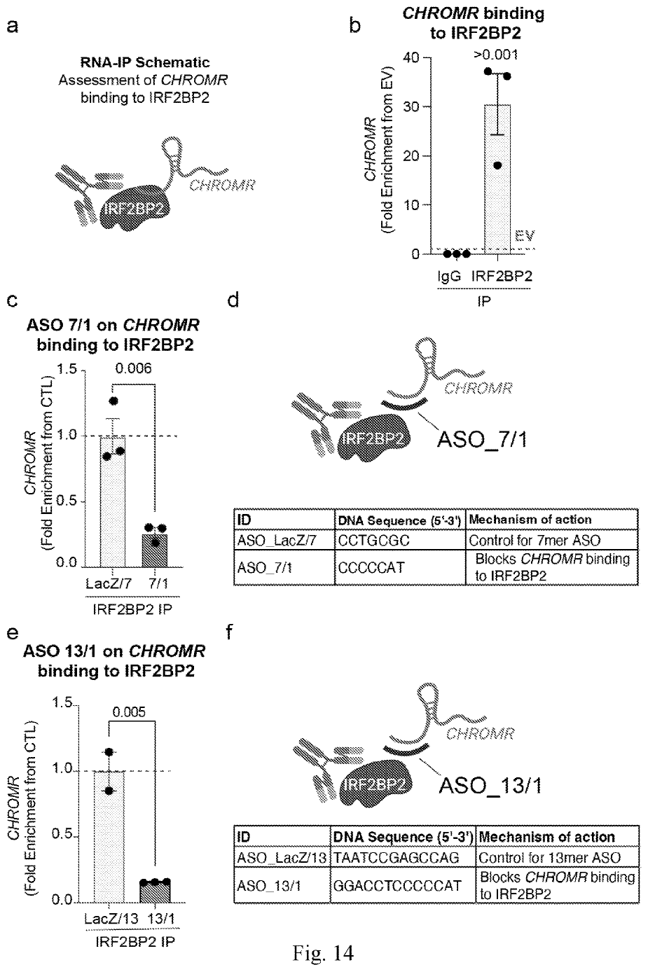

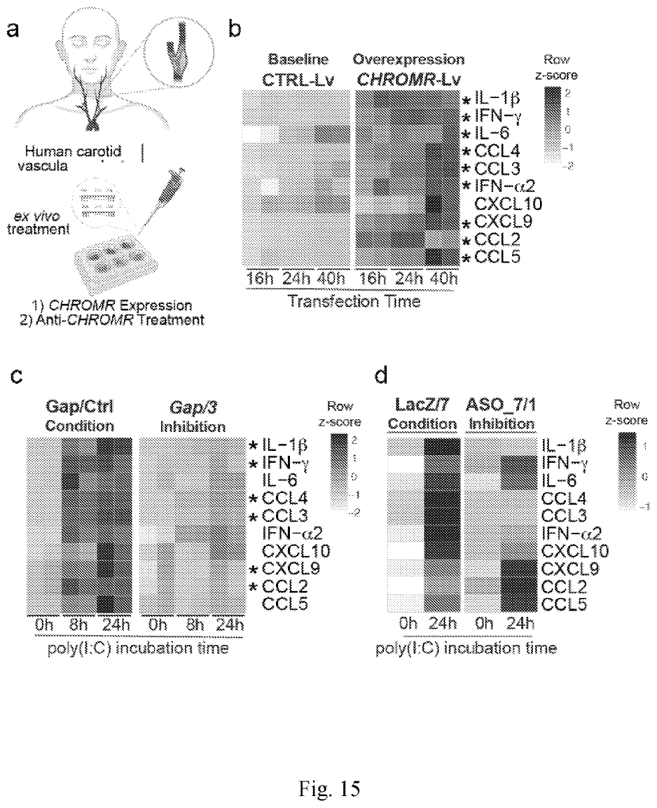

Sequences in FIG. 3, panel H. Predicted sequence, simplified from original analysis that contains a measure of the experimental variation in the detection of the nucleotides in each position. For clarity, most represented nucleotide is shown.

| HOMER analysis | |
|---|---|
| DNA Sequence (5′-3′, | |
| highest prediction) | Transcription factor |
| CGGTGACTCAGC (SEQ ID NO: 3) | BACH1/BACH2/FOSL2/AP-1/FOS |
| AAACCGAAAG (SEQ ID NO: 4) | IRF2/STAT2/IRF1/IRF8/ISRE |
| ACAACTTCCG (SEQ ID NO: 5) | ETV5/ETV6/ETS/ELF1/ELF4 |
| GGTCAGAGTT (SEQ ID NO: 6) | NRC2C2/TR4/THRB/PPARD |
| CCGGCCGGAC (SEQ ID NO: 7) | HINFP/E2F/KLF1/E2F6 |
| TCGTTCCGCC (SEQ ID NO: 8) | E2F6/EF2F1/HINFP/E2F3 |
| CAGACAGAACAC (SEQ ID NO: 9) | SMAD3/SMAD2/FOXJ3 |
| GCAGTCGGGGAC (SEQ ID NO: 10) | ZNF143/GCM1/HIC1 |
| TGTAATCGTAAAC (SEQ ID NO: 11) | GATA1/FOXL2/DUX4 |
| GCGGAAACGAAA (SEQ ID NO: 12) | IRF8/IRF7/ISRE |

FIG. 4. CHROMR binds to IRF2BP2 to control interferon-stimulated gene expression. (A) Schematic representation of Chromatin Isolation by RNA Purification (ChIRP) followed by genomic DNA sequencing (ChIRP-Seq) or mass spectrometry (ChIRP-MS) to identify RNA-binding proteins. (B) Distribution of CHROMR binding sites within interferon stimulated gene (ISG) loci (left) and representative ChIRP-seq reads (top: even probe set; middle: odd probe set; bottom: input) at selected ISG promoters (right). (C) Nuclear CHROMR-binding proteins identified by ChIRP-MS in THP-1 macrophages from 3 independent experiments. (D) Percentage of cells infected with influenza A virus/WSN/1933 (H1N1, 1000 PFU) in THP-1 macrophages transfected with siRNAs against IRF2BP2, IRF2 or a non-targeting siRNA control (siCTRL). (E) qPCR analysis of CHROMR3 in RNA-immunocomplexes precipitated from THP-1 macrophages using IRF2BP2 or HNRNPLL antibodies, or IgG as a control. (F) Representative microscopic image of RNA-FISH staining for CHROMR (red) in combination with immunofluorescent staining for IRF2BP2 in THP-1 macrophages. Merged image indicates signal colocalization (yellow). (G) catRAPID predicted interaction profile of IRF2BP2 with CHROMR3 or CHROMR3-G4 mutant (position of mutation indicated by ** in boxed region). (H) Visualization of CHROMR3 secondary structure using RNArtist, with the putative IRF2BP2-G-quadruplex interaction domain highlighted in red (bottom). Site-directed mutation of the putative G-quadruplex (underlined) in CHROMR3 (top). (I) Relative enrichment of CHROMR3 or CHROMR3-G4mut in MYC-IRF2BP2 immunoprecipitates. (J) Integrated model depicting CHROMR binding to IRF2BP2 to sequester the IRF-2 repressor complex from interferon-stimulated response elements (ISRE), facilitating access for activating interferon regulatory factors (e.g., IRF-1). (E, I) Data are relative to IgG control; mean±standard error of 3 independent experiments. P values were calculated using one-way ANOVA with Dunnett's multiple comparison test (D and I) or a repeated measures two-way ANOVA with Sidak's multiple comparison test (E). *P≤0.05; **P<0.01; ***P<0.001.

FIG. 4, panel H. CHROMR gene mutation in the G-quadruplex region between nucleotide 237 and 264. Mutated nucleotides are bolded.

| CHROMR Sequence | |
|---|---|
| (5′-3′, DNA) | ID |
| GGGGGAGGTCC | CHROMR variant 3 - |
| CAAAGCGGATA | Wild type |
| TCCTGG | (nt 237-264) |
| (SEQ ID NO: 13) | |
| GGGGGACCTCC | CHROMR variant 3 - |
| CAAAGCCCATA | G4mutant |
| TCCTGG | (nt 237-264) |
| (SEQ ID NO: 14) | |

FIG. 5. CHROMR expression levels in whole blood of patients infected with influenza A virus is associated with expression of cholesterol efflux genes and interferon-stimulated genes. (A) CHROMR expression (CPM) in whole blood of patients with influenza A virus (IAV) infection correlates positively with expression of cholesterol efflux genes ABCA1, ATP8B1, and CPT1A. (B) Pearson correlation matrix showing the 50 ISGs that are most strongly associated with CHROMR in whole blood in IAV-infected patients. (A) r=Pearson correlation coefficient, (B) Individual dot size and color represent Pearson correlation coefficient and absence of dot indicate lack of association. P<0.05.

FIG. 6. CHROMR is required for LPS induction of interferon-stimulated gene expression. (A) CHROMR expression (FKPM) in human monocyte-derived macrophages infected with influenza A/Vietnam/1203/2004 (H5N1), or mock infected. (B-C) qPCR analysis of CHROMR in THP-1 macrophages after transfection with CHROMR-targeting (GapCHROMR) and control GapmeRs (GapCTRL) (B) or in THP-1 macrophages stable overexpressing CHROMR or an empty vector control (C). (D) Volcano plot showing differential expression of genes identified by RNA-seq of THP-1 macrophages transfected with GapCHROMR versus GapCTRL and stimulated with LPS (500 ng/ml) for 3 h. Dashed lines indicate fold change (log 2)=+/−1; P-adj=0.05; red dots indicate upregulated genes; blue dots indicate downregulated genes; grey dots indicate non-significantly changed genes. (E) Hierarchical clustering heatmap showing normalized gene expression values in THP-1 macrophages transfected with GapCHROMR or GapCTRL and subsequently stimulated with LPS (500 ng/ml) for 3 h. Cutoffs used for visualization: −2<FC>2; and P-adj<0.05. (F, I and J) Volcano plots and tables of most affected canonical pathways (F), cytokines (I), and transcriptional regulators (J) identified through Ingenuity Pathway Analysis of (E); dashed lines indicate Z-score=+/−2 and P-adj=0.05; red dots indicate upregulated factors and blue dots indicate downregulated factors; non-significantly changed factors are indicated by grey dots. (G-H) Normalized expression counts (G) and protein levels (H) for CCL2, CCL5, CXCL10, and CXCL11 in THP-1 macrophages transfected with GapCHROMR or GapCTRL and subsequently stimulated with LPS (100 ng/mL) for 3 h (G) or 24 h (H). Data are mean+/−standard error of the mean for 2 (A) or 3 (D to G, I and J) independent experiments, or representative of 3 independent experiments (B, C and H). P values were calculated using a repeated measures two-way ANOVA with Sidak's multiple comparison test (A), right-tailed Fisher's exact test (F, I and J), or a two-tailed unpaired Student's t-test (B, C, G and H). *P<0.05; **P<0.01; ***P<0.001.

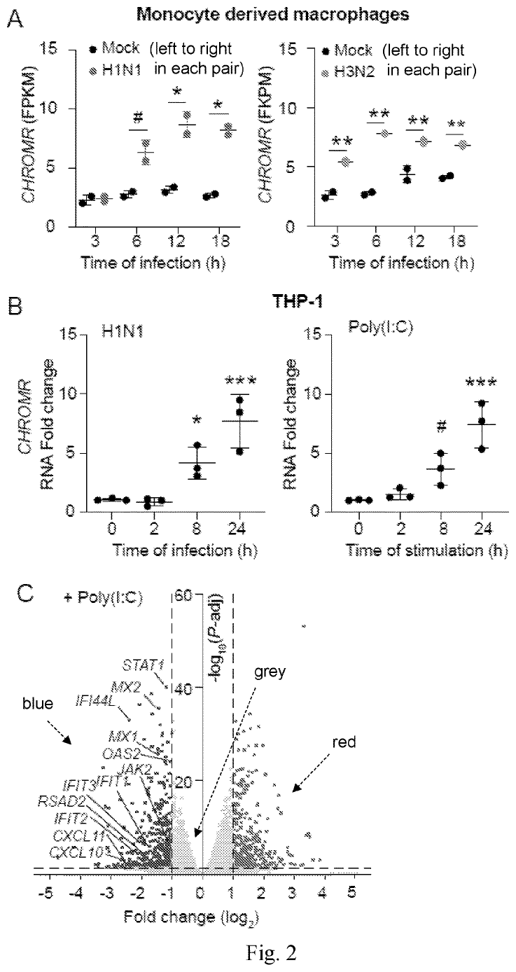

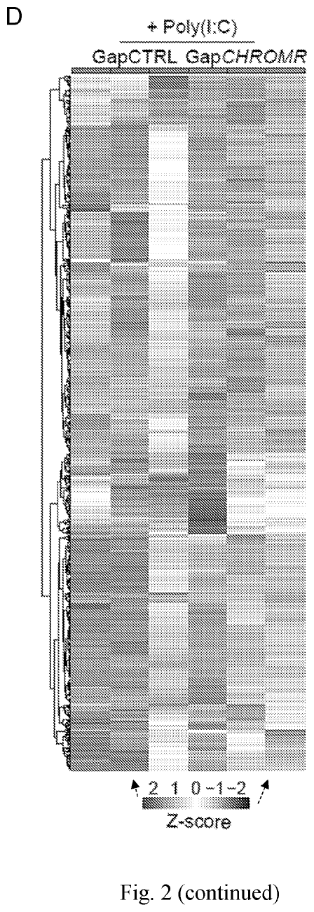

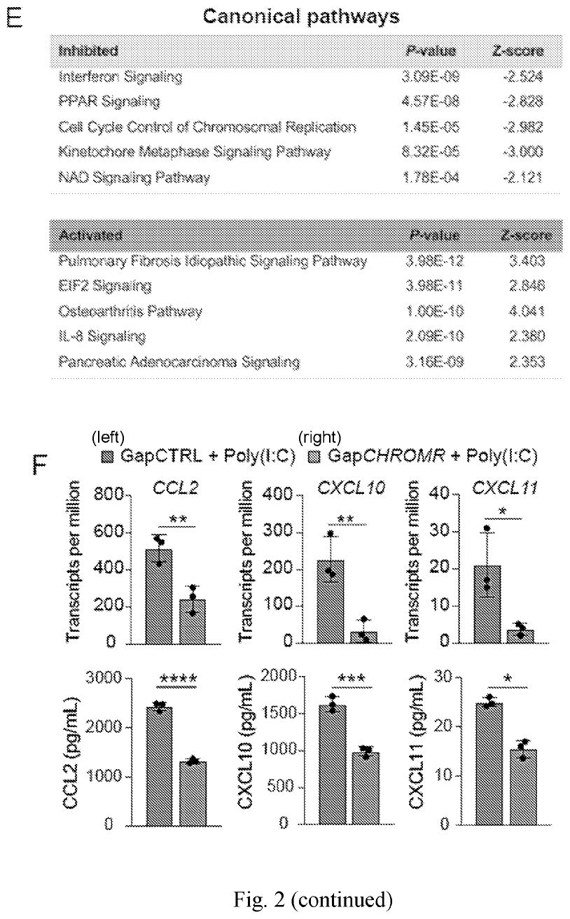

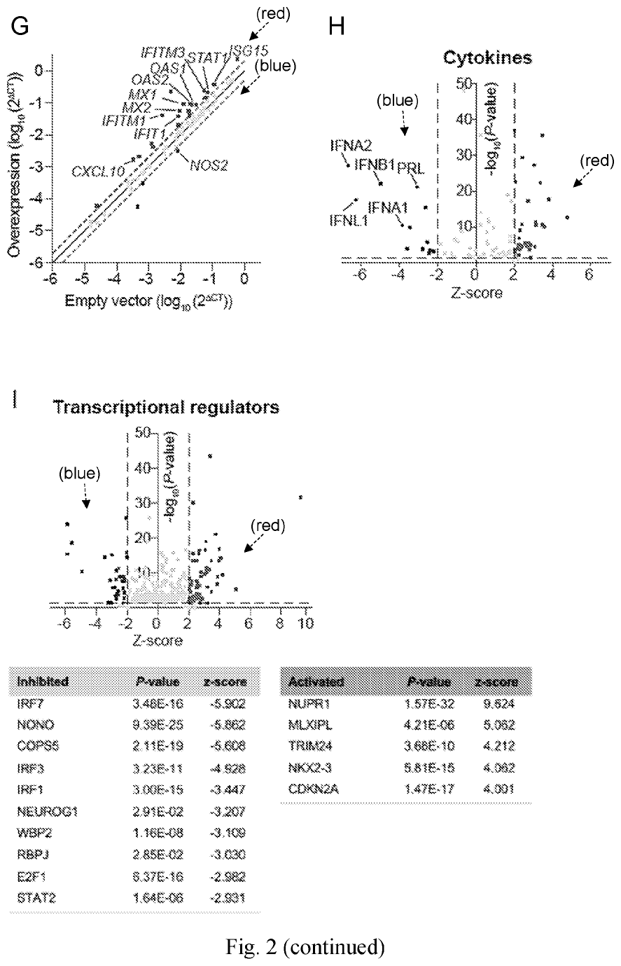

FIG. 7. CHROMR regulates ISG expression in response to LPS. Related to this figure is a constructed hierarchical clustering heatmap using Z-scores of differentially expressed interferon-stimulated genes in THP-1 macrophages transfected with GapCHROMR or GapCTRL and subsequently treated with LPS (500 ng/mL) for 3 h, P-adj<0.05 for which related data are described in Extended Data Table 4. (A) List of most affected microRNAs identified through Ingenuity Pathway Analysis of FIG. 2C. (B) Bar plots showing transcription factor binding enrichment analysis against the ChIP Enrichment Analysis (ChEA 2016) database gene set library using the inhibited genes (top) and activated genes (bottom) indicated in Extended Data Table 4. (C) Representative whole-well microscopy images of immunofluorescent staining for influenza-A-infected (H1N1, green) THP-1 macrophages transfected with GapCTRL or GapCHROMR and counterstained for nuclear RNA (DAPI, blue). P values were calculated using Binomial test (A-B).

FIG. 8. CHROMR is enriched in the nucleus and interacts with histones. (A) qPCR analysis of CHROMR variants after cellular fractionation of THP-1 macrophages. ActinB and lncRNA HOTAIR are used as cytoplasmic and nuclear controls, respectively. (B) Enrichment of CHROMR variants in H3-immunoprecipitates from THP-1 macrophages relative to IgG control. lncRNA NEAT1 was used as H3-enriched control. (C) Heatmap showing chromatin interactions at genomic location of CHROMR. (D) Pol II ChIA-PET analysis in K562 indicating chromatin interactions within genomic location indicated in (C) black, connected bars indicate direct interactions. (E) Zoom of topologically associated domain (TAD) containing CHROMR. Connected bars indicate direct interactions. (F) Normalized transcript reads (CPM) of genes present in CHROMR's TAD indicated in (D and E) from THP-1 macrophages transfected GapCHROMR or GapCTRL. Data (A, B, F) are mean+/−standard error of the mean for 3 independent experiments. P values were calculated using a repeated measures two-way ANOVA with Sidak's multiple comparison test (B). ***P<0.001; ****P<0.0001.

FIG. 9. Shown is (A) Identification of cytoplasmic CHROMR-binding proteins by ChIRP-MS (Comprehensive identification of RNA-binding proteins by mass spectrometry) in THP-1 macrophages. Mean score of 3 independent experiments. (B) QGRS-mapper analysis of putative Quadruplex forming G-Rich Sequences (QGRS, G-quadruplex) in CHROME3. Putative G-quadruplexes are highlighted in blue. (C) qPCR analysis of CHROME3 expression in HEK-293T cells transfected with plasmids expressing CHROME3, CHROME3-G4mut or control. Data are mean+/−standard error of the mean for 2 independent experiments (C). P values were calculated using a Student's t-test (C).

FIG. 9, panel B. Bioinformatic assessment of putative G-quadruplex in CHROMR. Identified GG-pairs are bolded.

| Position | Length | QGRS | G-score |
|---|---|---|---|
| 15 | 30 | GGCACAAAAAGCAGG | 11 |
| | | AACGAGTAGCGGTGG | |
| | | (SEQ ID NO: 15) | |
| 237 | 28 | GGGGGAGGTCCCAAA | 16 |
| | | GCGGATATCCTGG | |
| | | (SEQ ID NO: 13) | |
| 675 | 29 | GGCTGGTCTCAAAC | 11 |
| | | TTTTGGCCTCAAGGG | |
| | | (SEQ ID NO: 16) | |

FIG. 10. CHROMR expression correlates with SLE two-score IFN system signature and knocking down CHROMR expression by GapmeRs leads to reduced IRF transcriptional activity induced by SLE-relevant agonists Imiquimod. a) CHROMR levels correlate strongly with a clinically relevant scoring-system consisting of a module of IFN-stimulated genes in SLE patients (n=100), however these associations are absent in healthy controls (n=30), and b) CHROMR levels differ based on patient autoantibody status (n=4/status). c) GapmeR-mediated knock-out of CHROMR in an assay for IRF-driven transcription (luciferase, Relative units, RU) in THP-1 Dual reporter macrophages transfected with a control GapmeR (circles) or GapmeR against CHROMR (squares) and treated with TLR7 agonists Imiquimod (10 μg/mL). c) Relative expression normalized to baseline (0 h=100%). Data mean±standard error of the mean, n=3. P-value by repeated-measure 2-way ANOVA with Sidak's multiple comparison test. ***P<0.001.

FIG. 11. CHROMR-targeting 7-Mer (ASO_7/1) antisense oligonucleotides designed against the first GG-pair of a G-quadruplex present in CHROMR efficiently decrease IFNβ-induced ISG expression without causing CHROMR degradation. a) ASO_7/1 decreases IFNβ-induced (1000 U/ml) ISG expression in ISG-reporter human macrophages (Dual THP-1) and do not induce ISG expression on their own in the absence of IFNβ (vehicle condition). b) Expression of CHROMR upon treatment of macrophages with IFNβ or a vehicle solution (Veh.) is unaffected by treatment with ASO_7/1, as intended. c) Graphical representation showing which GG-pair of the G-quadruplex of CHROMR is targeted by individual 7-Mer antisense oligonucleotide (top), and their respective ASO DNA sequence as used in this experiment (bottom; ASO_7/1 is shaded to highlight that it is the only effective ASO). Data mean±standard error of the mean, n=3-4; P-value by 1Way ANOVA with multiple comparison test.

FIG. 12. CHROMR-targeting 13-Mer (ASO_13/1) antisense oligonucleotides designed against the first two GG-pairs of a G-quadruplex present in CHROMR efficiently decrease IFNβ-induced ISG expression without causing CHROMR degradation. a) ASO_13/1 decreases IFNβ-induced (1000 U/ml) ISG expression in ISG-reporter human macrophages (Dual THP-1) and do not induce ISG expression on their own in the absence of IFNβ (vehicle condition). b) Expression of CHROMR upon treatment of macrophages with IFNβ or a vehicle solution (Veh.) is unaffected by treatment with ASO_13/1, as intended. c) Graphical representation showing which GG-pairs of the G-quadruplex of CHROMR are targeted by individual 13-Mer antisense oligonucleotide (top), and their respective ASO DNA sequence as used in this experiment (bottom; ASO_13/1 is shaded to highlight that it is the only effective ASO). Data mean±standard error of the mean, n=3-4; P-value by 1Way ANOVA with multiple comparison test.

FIG. 13. CHROMR-targeting GapmeR (ASO_Gap/3) designed to cause CHROMR degradation efficiently decreases IFNβ-induced ISG expression and decreases CHROMR expression. a) ASO_Gap/3 decreases IFNβ-induced (1000 U/ml) ISG expression in ISG-reporter human macrophages (Dual THP-1) and does not induce ISG expression on its own in the absence of IFNβ (vehicle condition). b) Expression of CHROMR upon treatment of macrophages with IFNβ is decreased by treatment with ASO_13/1, as intended. c) GapmeR ASO DNA sequences as used in this experiment (ASO_Gap/3 is shaded to enhance clarity). Data mean±standard error of the mean, n=3-4; P-value by 1Way ANOVA with multiple comparison test (a) or Student's t-test (b).

FIG. 14. CHROMR-targeting antisense oligonucleotides designed against the first GG pair of a G-quadruplex present in CHROMR decrease binding to IRF2BP2. a) Graphical representation of an RNA immunoprecipitation (IP) experiment showing how antibodies against IRF2BP2 can be used to indirectly immunoprecipitate CHROMR and assess the extent of the binding between CHROMR and IRF2BP2; b) RNA-IP experiment showing that CHROMR binds to IRF2BP2 after overexpression of CHROMR and IRF2BP2 in human embryonic kidney (HEK293T) with empty overexpression vector (EV) used as control. c) Relative decrease in CHROMR enrichment on IRF2BP2 caused by ASO_7/1 targeting of CHROMR compared to their respective non-targeting ctrl (LacZ/7) by RNA-IP, with d) the graphical representation of the experiment (top) and DNA sequence of ASOs used in the experiment (bottom). e) Relative decrease in CHROMR enrichment on IRF2BP2 caused by ASO_13/1 targeting of CHROMR compared to their respective non-targeting ctrl (LacZ/13) by RNA-IP, with f) the graphical representation of the experiment (top) and DNA sequence of ASOs used in the experiment (bottom). Data is mean±standard error of the mean; P-value by Student's t-test

FIG. 15. CHROMR-targeting ASO are efficient at reducing cytokine secretion in human vascular explants. a) Human carotid vascular explant model for testing CHROMR-ASO function ex vivo; b) Heatmap showing row Z-score of secretion levels of selected cytokine/chemokine measured by Luminex-immunoassay from supernatant of vascular explants transfected with control (CTRL-Lv) or CHROMR overexpressing vector (CHROMR-Lv), or c-d) transfected with GapmeRs targeting CHROMR (gapCHROMR) and control (gapCTRL) (c) or the G-quadruplex-targeting 7-Mer ASO (d) and stimulated with TLR3 agonist (poly[I:C]; 1 μg/ml) to stimulate an inflammatory response. P-value by RM-2Way ANOVA with multiple comparison. *P<0.05; n=1-2.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide sequences described herein. Complementary and anti-parallel polynucleotide sequences are included.

As used in the specification and the appended claims, the singular forms “a” “and” and “the” include plural referents unless the context clearly dictates otherwise. Ranges and other values may be expressed herein as from “about” or “approximately” one particular value, and/or to “about” or “approximately” another particular value. When values are expressed as approximations by the use of the antecedent “about” or “approximately” it will be understood that the particular value forms another embodiment. The term “about” and “approximately” in relation to a numerical value encompasses variations of +/−10%, to +/−1%.

The disclosure provides agents that are used for prophylaxis or treatment of disorders that are associated with type I interferon induced inflammation. In embodiments the agent comprise an antisense oligonucleotide targeted to CHROMR RNA. Representative examples of olignoucleotides that function in the described methods are provided, as are examples of olignoucleotides that also target CHROMR RNA but do not function, or do not function as efficiently, as the oligonucleotides that are used in the described methods. In embodiments, a described oligonucleotide comprises or consists of 7-20 nucleotides. Any oligonucleotide described herein can comprise or consist of a described sequence.

Any antisense oligonucleotide of this disclosure may be referred to herein as “ASO.” Any antisense oligonucleotide described herein may modified or unmodified. As such, the terms “oligonucleotide” and “antisense oligonucleotide” and “ASO” as used herein includes unmodified oligonucleotides and modified oligonucleotides that include modified nucleotides and/or modified nucleotide linkages, including but not necessarily limited to methylation. The nucleotides of the oligonucleotides may be nucleotide analogs. Modified nucleotides that can be incorporated into the described antisense oligonucleotides are known in the art, such as those described in Metelev V G, Oretskaya T S. Modified Oligonucleotides: New Structures, New Properties, and New Spheres of Application. Russ J Bioorg Chem. 2021; 47 (2): 339-343. doi: 10.1134/S1068162021020175. Epub 2021 Apr. 2, the disclosure of which is incorporated herein by reference. The nucleotides or nucleotide analogs may be linked by phosphodiester linkages or by a synthetic linkage, i.e., a linkage other than a phosphodiester linkage. Non-limiting examples of linkages in the modified oligonucleotide agents that can be used in this disclosure include phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. Thus, in embodiments, any oligonucleotide described herein may be provided as a GapmeR. Oligonucleotides that comprise DNA/RNA hybrids are included.

In non-limiting embodiments, oligonucleotides that are used in the described methods comprise the sequence CCCCCAT, which may also be referred to herein as ASO_7/1. In non-limiting embodiments an oligonucleotide used in the described methods comprises the sequence GGAGGTCCCCCAT (SEQ ID NO: 2), which may also be referred to herein as ASO_13/1. In non-limiting embodiments an oligonucleotide used in the described methods comprises the sequence CTCATAAGAAAACTGA (SEQ ID NO: 1), which may also be referred to herein as ASO_Gap/3.

As described further below by way of the Examples, an oligonucleotide of this disclosure may exhibit one or several functions that are involved in prophylaxis or treatment of disorders that are associated with type I interferon induced inflammation. In non-limiting embodiments, an ASO of this disclosure can inhibit expression of one or more interferon-stimulated genes (ISGs). In non-limiting embodiments, an ASO of this disclosure prevents or inhibits CHROMR from binding to Interferon Regulatory Factor-2 Binding Protein 2 (IRF2BP2). In embodiments, an ASO of this disclosure may be functional in a described method and participate in degradation of CHROMR. In embodiments, an ASO of this disclosure may be functional in a described method without participating in degradation of CHROMR. In embodiments, the described oligonucleotides exert their effects on CHROMR in the nucleus, in contrast to previous descriptions of the function of RNA CHROMR that regulates cholesterol efflux and fatty acid oxidation via microRNA sequestration in the cytoplasm.

In embodiments, a composition comprising an antisense oligonucleotide of this disclosure is administered to an individual in a therapeutically effective amount. The term “therapeutically effective amount” as used herein refers to an amount of a described agent sufficient to achieve, in a single or multiple doses, the intended purpose of treatment. The amount desired or required may vary depending on the particular oligonucleotide or combination of oligonucleotides used, the mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. A precise dosage can be selected by in view of the patient to be treated. Dosage and administration can be adjusted to provide sufficient levels of components to achieve a desired effect. Factors which may be taken into account include the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse.

In embodiments, the individual in need of a described oligonucleotide has any condition that is associated with type I interferon induced inflammation, including but not necessarily limited to an autoimmune disorder or a type I interferon induced inflammation that is associated with a pathology of the cardiovascular system of the individual. In embodiments the type I interferon induced inflammation is associated with or causes a type I interferonopathy. In embodiments the type I interferon induced inflammation is associated with a hyperactive type I interferon (IFN) response.

In embodiments, the individual in need of a described oligonucleotide has an autoimmune disorder that is any of Systemic lupus erythematosus (SLE), psoriasis, type I diabetes, rheumatoid arthritis, Sjögrens syndrome, dermatomyositis, Aicardi-Goutières Syndrome, Familial chilblain lupus, STING-associated vasculopathy spastic paraparesis, Singleton-Merten syndrome, Trichohepatoenteric syndrome, infantile encephalopathy, ataxia telangiectasia, Bloom syndrome, common variable immunodeficiency, or proteasome-associated autoinflammatory syndrome.

In embodiments, the individual in need of a described oligonucleotide has type I interferon induced inflammation that is associated with pathology of the cardiovascular system, such as comprises heart failure. In embodiments the heart failure has been induced by treating the individual with interferon-β, e.g., of iatrogenic origin, or of unknown origin, or is a sepsis-induced cardiomyopathy.

In embodiments, the individual does not have a condition associated with cholesterol efflux or high-density lipoprotein (HDL) biogenesis. In embodiments, the individual does not have a disorder that is associated with elevated cholesterol, or cholesterol homeostasis. In embodiments, the individual who is treated with a described oligonucleotide is not also treated with an inhibitor or any other non-coding RNA, including but not necessarily limited to miR-33.

In embodiments, the described oligonucleotides are provided in the form of a pharmaceutical formulation. A pharmaceutical formulation can be prepared by mixing the compound(s) with any suitable pharmaceutical additive, buffer, and the like. Examples of pharmaceutically acceptable carriers, excipients and stabilizers can be found, for example, in Remington: The Science and Practice of Pharmacy (2020) 23rd Edition, Academic Press, the disclosure of which is incorporated herein by reference. In embodiments a described ASO is used with nanoparticles, including but not necessarily limited to liposomal formulations.

Administration of pharmaceutical formulations comprising the described oligonucleotides of this disclosure can be performed using any suitable route of administration, including but not limited to parenteral, intraperitoneal, and oral administration. Parenteral infusions include intramuscular, intravenous, intraarterial, intraperitoneal, and subcutaneous administration. In embodiments, a single administration is administered and is sufficient for a therapeutic response. In embodiments, more than one administration is provided. In embodiments only a single administration is used. In embodiments, only one, or only a combination of oligonucleotides described herein are used in a described method. Thus, in embodiments, a described oligonucleotide or combination of described oligonucleotides may be the only therapeutic agent(s) used in a described method. Notwithstanding, in embodiments one or more described oligonucleotides may be combined with standard anti-inflammatory agents, such as anti-inflammatory steroids, selective or non-selective non-steroidal anti-inflammatory drugs (NSAIDs), and the like.

The following Examples are intended to illustrate embodiments of the disclosure but are not intended to be limiting.

### Example 1

LncRNA CHROMR Associates with the Interferon Response in Patients with COVID-19 and Influenza

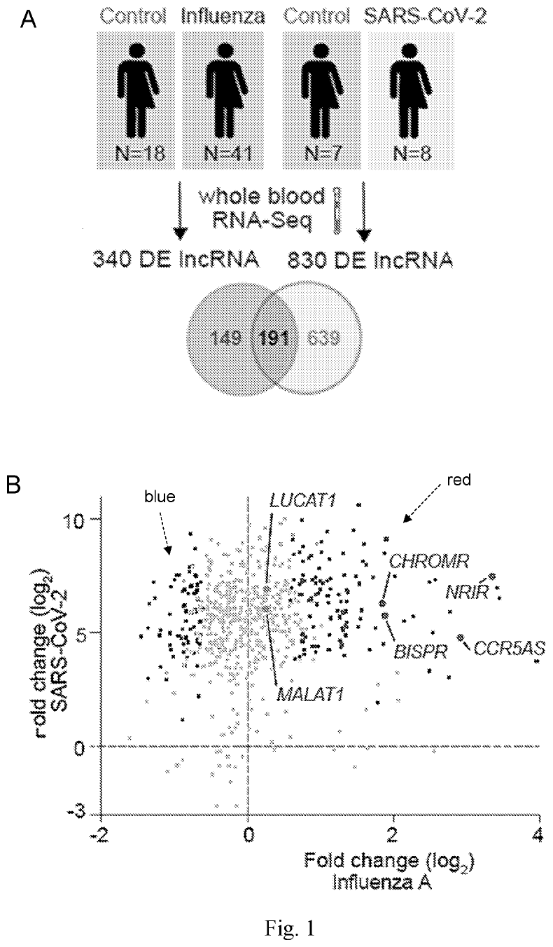

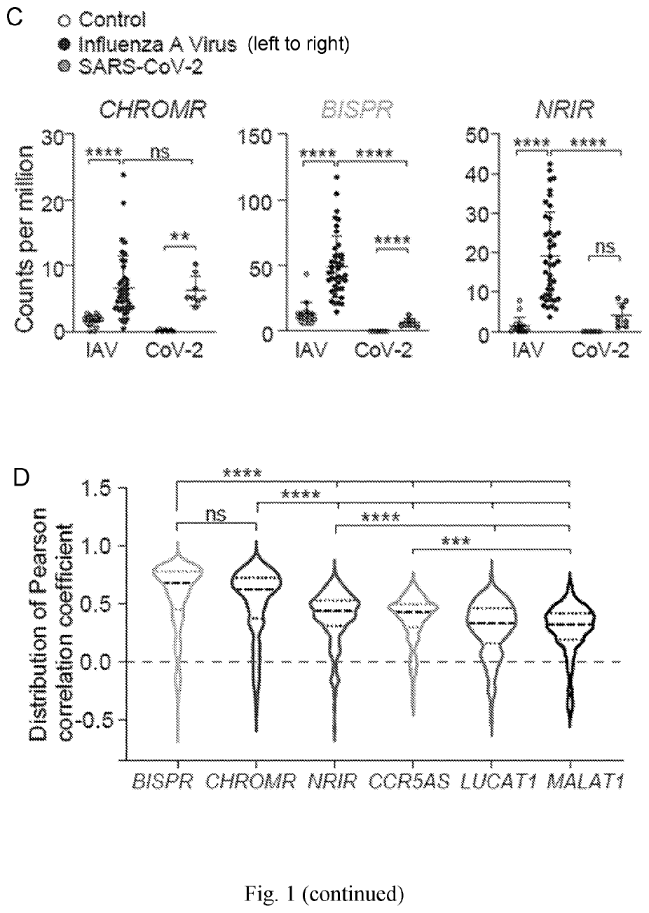

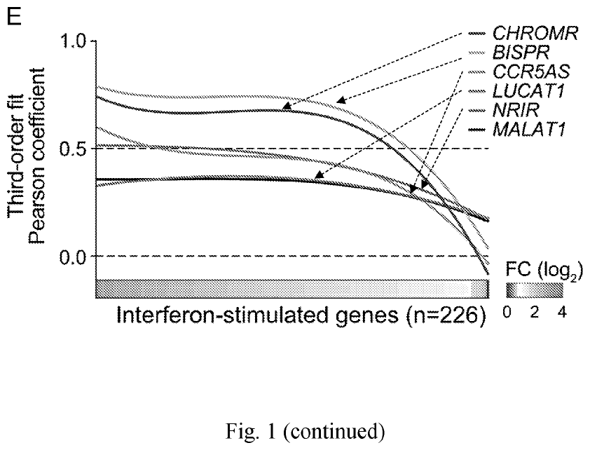

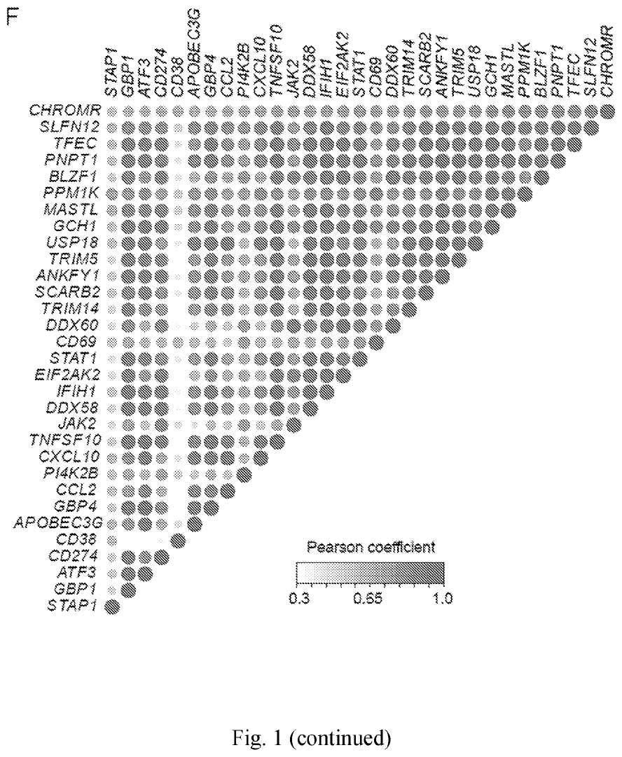

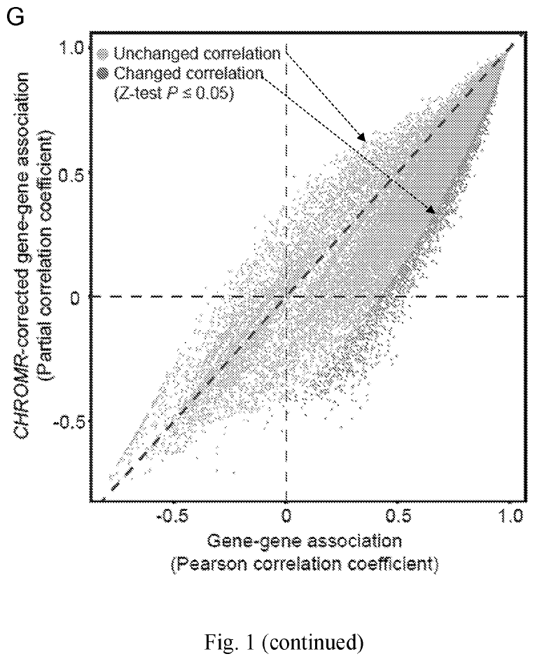

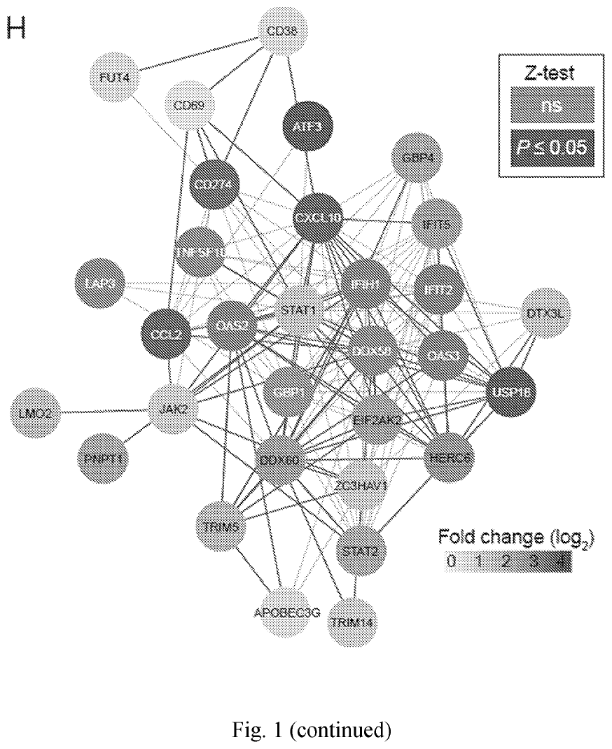

To identify lncRNAs implicated in the host response to respiratory viruses, we performed RNA sequencing (RNA-seq) of whole blood from hospitalized patients with coronavirus disease 2019 (COVID-19) induced by SARS-CoV-2 (n=8) and age- and sex-matched controls (n=7) (Extended Data Table 1), and compared this to whole blood transcriptomic analysis of subjects with influenza A virus (n=41) and controls (n=18) (retrieved from⁽²¹⁾). Differential expression analysis revealed 830 lncRNAs altered in patients infected with SARS-CoV-2 and 340 changed in patients with influenza A infection; 191 lncRNAs were dysregulated in both diseases (P-adj<0.05, −1.5<fold change>1.5; FIGS. 1A and B and Extended Data Table 2). Among the top mutually upregulated lncRNAs, we identified CHROMR (alias CHROME) (FIGS. 1B and C), a primate-specific lncRNA previously identified to regulate cellular lipid metabolism⁽²⁰⁾. Of note, levels of CHROMR strongly correlated with ISGs differentially expressed (n=226) in COVID-19 and influenza patients compared to controls (FIG. 1D), in addition to previously associated lipid metabolism genes (FIG. 5A). When compared to other human lncRNAs known to regulate antiviral responses^{(14-16, 19, 22)}, CHROMR showed a distribution of correlation coefficients equivalent to BISPR (IncBST2) and significantly higher than NRIR, CCR5AS, LUCAT1 and MALAT1 (FIG. 1D). Of these lncRNAs, only CHROMR showed an equivalent transcriptional response to both viral infections (FIG. 1C). To further visualize the association of CHROMR with ISGs differentially expressed after influenza A or COVID-19 infection, we rank-ordered the ISGs by level of differential expression in influenza A-infected patients and plotted their correlation coefficient with CHROMR. Using a robust third-order non-linear regression analysis, we observed that CHROMR associates strongly with genes that are upregulated by ≥2-fold change (mean r≥0.5), similar to that observed with BISPR, whereas CCR5AS, NRIR, LUCAT1, and MALAT1 did not exhibit a distinct pattern of association with the continuum of differentially expressed ISGs (FIG. 1E). Among the top 30 ISGs most correlated with CHROMR, we observed that 453 of 465 ISG×ISG pairs are significantly associated (FIG. 1F). To investigate whether CHROMR influences ISG×ISG associations as a covariate, we compared the bivariate correlation coefficient of all 226 differentially expressed ISGs to their corresponding CHROMR-corrected partial correlation coefficient. The majority of the 25,425 ISG×ISG correlation coefficients generated were decreased by CHROMR correction, with 1,845 significantly decreased (Fisher's r-to-Z transformation followed by Z-test; FIG. 1G). Of note, only one ISG×ISG correlation coefficient was significantly increased, indicating that CHROMR has a robust positive impact on the correlation between ISGs. We next generated a functional interactome of the 50 genes whose expression is most associated with CHROMR (FIG. 5B). Of these, 31 genes were functionally related, representing a total of 172 ISG×ISG associations (all edges, FIG. 1H), 79 of which were statistically altered upon correction for CHROMR (blue-colored edges, FIG. 1H). Taken together, these results indicate a role for CHROMR in directing the coordinated ISG response to SARS-CoV-2 and influenza A infection.

To understand how CHROMR is regulated in myeloid cells during viral infection, we interrogated RNA-seq data from human monocyte-derived macrophages infected with A/California/04/09 (H1N1), influenza A/Wyoming/03/03 (H3N2) or influenza A/Vietnam/1203/2004 (H5N1) HaLo viruses (retrieved from⁽²³⁾). We observed that CHROMR expression was increased within 3-6 hours after influenza A virus (IAV) infection compared to mock treatment (FIG. 2A and FIG. 6A). Similar findings were observed in human THP-1 monocyte-derived macrophages infected with influenza A virus/WSN/1933 (H1N1) virus or treated with the viral mimic poly(I:C), a synthetic double-stranded RNA that activates TLR3 (FIG. 2B). To assess the impact of CHROMR depletion on the transcriptional response to innate immune stimulation, we knocked down CHROMR in THP-1 macrophages using GapmeR antisense oligonucleotides (GapCHROMR) or control GapmeRs (GapCTRL) and treated with poly(I:C) for 8 hours. We confirmed that CHROMR expression was diminished upon treatment with GapCHROMR when compared to GapCTRL by quantitative real-time PCR (qPCR) (FIG. 6B). Transcriptome profiling by RNA-seq and unsupervised hierarchical clustering of genes differentially expressed in CHROMR-sufficient and -depleted macrophages revealed that CHROMR silencing markedly reprogrammed transcriptional responses to poly(I:C), with 488 genes showing lower transcript levels and 395 genes showing higher transcript levels compared to GapCTRL treated cells (−2<fold change>2, P-adj<0.05; FIGS. 2C and D). Ingenuity Pathway Analysis of genes differentially expressed upon CHROMR knockdown identified ‘Interferon signaling’ as the most repressed canonical pathway, followed by ‘PPAR signaling’ and ‘Cell cycle control of replication’ (FIG. 2E). The most differentially regulated genes included the antiviral response genes IFIT1, IFIT2, IFIT3, RSAD2, MX1, MX2, IFI44L, and STAT1 (FIG. 2C and Extended Data Table 3). In addition, we noted that transcript levels of several IFN-induced chemokines were reduced in CHROMR depleted macrophages treated with poly(I:C), including members of the CXCL (CXCL10, CXCL11) and CCL (CCL2) families, which was further validated at the protein level by bead-based immunoassay (FIG. 2F). A similar downregulation of ISGs was observed in CHROMR-depleted macrophages treated with bacterial lipopolysaccharide (LPS) (FIGS. 6D-H and Extended Data Table 4). Conversely, overexpression of CHROMR in THP-1 macrophages increased expression of ISGs, including CXCL10, IFIT1, IFITM1, IFITM3, MX1, MX2, OAS1, OAS2, STAT1, and ISG15 (FIG. 2G and FIG. 6C and Extended Data Table 5), as assessed using a qPCR array to profile 84 selected ISGs.

To identify potential factors driving CHROMR-associated transcriptional changes, we used Ingenuity Pathway Analysis to ascertain upstream regulators of genes differentially expressed upon CHROMR knockdown, including cytokines and transcription factors shown experimentally to alter the affected gene pathways. This analysis suggested repression of interferons, including type I (IFNα, IFNβ) and type III (IFNλ), in CHROMR-depleted THP-1 macrophages stimulated with poly(I:C) and LPS (FIG. 2H and FIG. 6I). Accordingly, transcription factors associated with interferon signaling were predicted to be inhibited upon CHROMR knockdown, including Interferon Regulatory Factors (IRF-1, IRF-3, IRF-7), and Signal Transducers and Activators of Transcription (STAT) proteins, which transduce signaling from the IFN receptors (FIG. 2I and Extended Data Table 3). Consistent with the established roles of IRF-1 and JAK-STAT signaling in the transcriptional regulation of ISGs, we noted that 115 of 389 ISGs induced by poly(I:C) and 230 of 389 ISGs induced by LPS were expressed at lower levels in GapCHROMR-compared to GapCTRL-treated THP-1 macrophages (Extended Data Table 4). Prominent among these ISGs downregulated upon CHROMR knockdown were genes important for pathogen sensing (TLR3, DDX58/RIG-I, IFIH1/MDA5, IFI16), as well as inhibition of viral entry (MX1, IFITM1, IFITM2, TRIM5), replication (IFIT, ISG15, OAS1) and budding (RSAD2/Viperin, BST2/Tetherin). As these data suggest CHROMR is an important component of the antiviral response, we next assessed the impact of CHROMR knockdown on influenza replication. THP-1 macrophages were treated with GapCHROMR or GapCTRL, and challenged with influenza A/WSN/1933 (H1N1) at increasing doses of 100, 500 or 1000 plaque forming units (PFU) per well for multi-cycle replication. CHROMR knockdown significantly increased IAV infection levels in GapCHROMR-compared to GapCTRL-treated macrophages, suggesting an important role for CHROMR in restricting IAV infection (FIG. 3A and FIG. 7C).

### Example 2

CHROMR Associates with Chromatin and Shapes H3K27Ac at ISG Regulatory Regions

As CHROMR is known to regulate lipid metabolism by sequestering microRNAs in the cytoplasm⁽²⁰⁾, we performed in silico analyses to predict microRNA regulators of the ISGs differentially expressed upon CHROMR-knockdown (FIG. 7A). miR-21 and miR-184 were identified as putative repressors of genes whose expression was reduced in GapCHROMR-compared to GapCTRL treated macrophages, however, CHROMR lacks binding sites for these microRNAs, suggesting an alternative mechanism of gene regulation. Cell fractionation studies revealed that CHROMR localizes to the nucleus as well as the cytoplasm of macrophages (Extended Data Table 4). Thus, we assessed whether CHROMR associates with chromatin by performing RNA immunoprecipitation of histone H3. qPCR of H3 immunoprecipitates showed enrichment of CHROMR, particularly variants 1, 3 and 4, at levels similar to another chromatin-binding lncRNA NEAT1⁽²⁴⁾ (FIG. 8B). As many nuclear lncRNAs can act in cis to regulate adjacent loci, we considered whether CHROMR could regulate genes within its topologically associating domain (TAD) (FIG. 8C). Chromatin Interaction Analysis by Paired-End Tag Sequencing, which combines chromatin immunoprecipitation (ChIP)-based methods, chromatin proximity interaction and chromosome conformation capture (3C) revealed weak interactions between CHROMR and its neighboring genes, including the PRKRA gene that encodes Protein ACTivator of the interferon-induced protein kinase PKR (PACT), which binds dsRNA and activates RIG-I-mediated antiviral signaling (FIGS. 8D and E). However, the frequency of those interactions was only marginally higher than that observed with genes in distant TADs, suggesting that CHROMR's TAD is not highly insulated. Furthermore, we observed no difference in the expression of genes within CHROMR's TAD, including PRKRA, in GapCHROMR and GapCTRL treated THP-1 macrophages, discounting gene regulation in cis as the mechanism of CHROMR's effect on interferon signaling (FIG. 8F).

To further investigate the role of CHROMR in transcriptional activation of ISGs, we performed gene set enrichment analysis of ISGs differentially expressed in GapCHROMR- and GapCTRL-treated macrophages stimulated with poly(I:C) or LPS using the chromatin immunoprecipitation-X enrichment analysis (ChEA) gene set library⁽²⁵⁾. ISGs downregulated upon CHROMR-depletion and subsequent microbial stimulus were most significantly enriched in ChIP experiments for IRF-1 (FIG. 3B and FIG. 7B), a transcription factor that regulates constitutive expression of antiviral genes and induction of the early antiviral response⁽²⁶⁾. Our analysis also identified the nuclear hormone receptor NR1H3/LXRA that controls transcription of lipid homeostasis genes previously linked to CHROMR⁽²⁷⁾. To test whether CHROMR regulates IRF activation, we employed THP-1 macrophages stably expressing IRF- and NF-κB-inducible reporter genes. While we observed no difference in NF-κB reporter activation in GapCHROMR or GapCTRL-treated macrophages, CHROMR knockdown reduced IRF reporter activity in both unstimulated and poly(I:C)-stimulated THP-1 macrophages (FIG. 3C). Since these studies suggested a defect in constitutive and induced IRF-activation in the absence of CHROMR, we next assessed the genome-wide distribution of histone H3 lysine 27 acetylation (H3K27Ac), an epigenetic mark of transcriptional activity, in THP-1 macrophages treated with GapCHROMR or GapCTRL. ChIP-sequencing (ChIP-seq) showed that CHROMR knockdown resulted in depletion of H3K27Ac at 2,753 genomic sites and enrichment of H3K27Ac at 30 sites compared to control macrophages (FIG. 3D). Classification of the H3K27Ac peak distribution among genomic features showed that the depletion of H3K27Ac marks after CHROMR knockdown occurred mainly in promoter regions (66%), followed by distal intergenic and intronic regions (FIG. 3E). Genomic Regions Enrichment of Annotations Tool (GREAT) analysis of genes exhibiting decreased H3K27Ac revealed enrichment of biological processes related to antiviral immunity, including ‘Response to type I interferon’, ‘Defense response to virus’, and ‘Negative regulation of viral life cycle’ (FIG. 3F). Indeed, characterization of the H3K27Ac read distribution across the transcriptional start site (±1,500 bp) of all ISGs showed markedly reduced H3K27Ac read density in GapCHROMR-versus GapCTRL-treated macrophages (FIG. 3G), consistent with lower transcription of ISGs in the absence of CHROMR. H3K27Ac helps shape active promoters and enhancers by opening chromatin to allow binding of transcriptional regulators. Notably, Hypergeometric Optimization of Motif EnRichment (HOMER) analysis of transcription factor binding motifs within regions of decreased H3K27Ac after CHROMR knockdown identified an ISRE motif predicted to bind IRF-1 and its functional antagonist IRF-2 (FIG. 3H), suggesting that CHROMR may shape active ISG promoters by facilitating IRF-1 recruitment.

### Example 3

### CHROMR Binds IRF-2 Binding Protein 2

To examine the possibility that CHROMR acts in trans to regulate expression of ISGs, we performed Chromatin Isolation by RNA Purification (ChIRP) from nuclear extracts of crosslinked THP-1 macrophages using 2 independent pools of biotinylated CHROMR-specific antisense RNA probes (FIG. 44). Isolation of CHROMR-associated chromatin followed by DNA sequencing (ChIRP-seq) revealed enrichment of CHROMR at 237 of 389 known ISGs, most prominently within promoter and intronic regions (FIG. 4B). As examples, genomic regions near the TSS of CXCL10, OAS2, and MX1, genes that were particularly affected by CHROMR gain- or loss-of-function (FIGS. 2C and G), showed binding of both pools of CHROMR-specific probes (FIG. 4B). These findings suggest that CHROMR binds, either directly or indirectly, to regulatory regions of ISGs to promote their transcription. To better understand how CHROMR might mediate this effect, we performed Comprehensive Identification of RNA-binding Proteins by Mass Spectrometry (ChIRP-MS)⁽²⁸⁾ in THP-1 macrophages to identify CHROMR-interacting proteins. We identified 26 proteins that co-precipitated with CHROMR, including 7 nuclear proteins, 14 cytoplasmic proteins, and 5 proteins that localized to both the nucleus and cytoplasm (FIG. 4C and FIG. 9A). Among the nuclear proteins associated with CHROMR were two factors previously implicated in the regulation of interferon responses: Topoisomerase 2a (TOP2A)⁽²⁹⁾ and Interferon Regulatory Factor-2 Binding Protein 2 (IRF2BP2)^{(30, 31)}. IRF2BP2 is a binding partner of the IRF-2 transcriptional repressor that antagonizes IRF-1-mediated transcriptional activation^{(30, 32)}. Thus, we postulated that CHROMR may regulate transcriptional activation of ISGs and antiviral responses by sequestering IRF2BP2. Consistent with this possibility, siRNA-mediated knockdown of endogenous IRF2BP2 or IRF-2 inhibited infection with influenza A/WSN/1933 (H1N1) compared to control siRNA treatment in THP-1 macrophages (FIG. 4D). The CHROMR and IRF2BP2 interaction was confirmed by RNA immunoprecipitation, which showed that CHROMR was enriched in IRF2BP2 immunoprecipitates compared to IgG controls (FIG. 4E). To further substantiate this interaction, we combined RNA fluorescence in situ hybridization for CHROMR with immunofluorescence for IRF2BP2 in THP-1 macrophages and observed nuclear colocalization (FIG. 4F). Using the catRAPID algorithm⁽³³⁾, we calculated the binding propensity of CHROMR and IRF2BP2, and predicted protein-binding regions within CHROMR. This analysis identified nucleotides 90-141 (domain 1), 177-269 (domain 2), and 468-552 (domain 3) of CHROMR as potential IRF2BP2 interaction domains (FIG. 4G). Notably, domain 2 of CHROMR contains tandem G-rich sequences predicted by QGRSmapper⁽³⁴⁾ to form a G-quadruplex (G4) secondary structure (FIG. 9B), which can enable RNA-protein interactions⁽³⁵⁾. In silico mutation of this putative G4, simulated by replacing two of the four tandem GG doublets with CC doublets, reversed the binding propensity of IRF2BP2 to domain 2 of CHROMR (FIG. 4G, right panel). To directly test whether CHROMR interacts with IRF2BP2 through this putative G4, we performed site-directed mutagenesis of two G-doublets into CC-doublets in CHROMR3 (FIG. 4H), and overexpressed wildtype (WT) or G4-mutant CHROMR with a MYC/DDK-tagged IRF2BP2 in HEK293T cells. We immunoprecipitated IRF2BP2 using an antibody against MYC/DDK, and assessed the presence of CHROMR in these complexes by qPCR. Although WT and G4-mutant CHROMR were expressed at similar levels (FIG. 9C), only WT CHROMR was enriched in IRF2BP2 immunoprecipitates (FIG. 4I), indicating that the G-rich sequence in domain 2 is required for CHROMR-IRF2BP2 binding.

These data indicate an important role for CHROMR in coordinating ISG expression and antiviral immunity in humans. By combining human transcriptomic profiling in influenza A and SARS-CoV-2 infection with in vitro mechanistic studies, we demonstrate that CHROMR regulates the antiviral gene program by sequestering the nuclear IRF-2/IRF2BP2 repressor complex thereby releasing its inhibitory effect on transcription of ISGs (FIG. 4J). These findings expand CHROMR's role as a competing endogenous RNA that regulates cholesterol efflux and fatty acid oxidation via microRNA sequestration in the cytoplasm. Many viruses rewire host lipid synthesis and metabolism to facilitate replication^{(36, 37)}, and thus, increased CHROMR expression in virus infected cells would both mitigate cellular lipid accumulation and increase ISG transcription to mount an antiviral immune response. CHROMR is not conserved in common preclinical animal models used to study antiviral immunity, whereas the present disclosure includes analysis of human responses.

### Example 4

The following materials and methods were used to produce the result in the Examples above.

Human studies. A cohort of eight hospitalized COVID-19 patients were recruited from NYU Langone Health between May 11-21, 2020. SARS-CoV-2 infection was confirmed by qPCR, in accordance with current standards. All COVID-19 patients and, age-, and sex-matched control donors were recruited under study protocols approved by the NYU Langone Health Institutional Review Board. No exclusion criteria were applied. Each study participant or their legal authorized representative gave written informed consent for study enrollment in accordance with the Declaration of Helsinki. For COVID-19 patients, enrollment criteria included age greater than 18, hospital admission, positive SARS-CoV-2 testing, and informed consent. COVID-19 patients were monitored until discharge or death. Demographics of the cohort are listed in Extended Data Table 1.

Whole blood transcriptome profiling. Whole blood of COVID-19 patients (n=8) and controls (n=7) was collected into PAXgene Blood RNA tubes (PreAnalytiX GmbH, BD Biosciences), and RNA was isolated. The quality and yield of the isolated RNA was determined with an Agilent 2100 Bioanalyzer (Agilent) prior to RNA-sequencing (RNA-seq). RNA library preps were made (Low input Clontech SMART-seq) and sequences as single-end mode at the Genome Technology Center at NYU Langone Health using Illumina NovaSeq6000.

Transcriptomic analysis. To obtain differentially expressed transcripts between influenza A infected patients (n=41) and controls (n=18) we queried publicly available dataset GSE157240⁽²¹⁾. FASTQ files from RNA-seq (influenza A and SARS-CoV-2) were processed using the Seq-N-Slide pipeline⁽³⁸⁾. Reads were aligned to the hg38 genome using STAR⁽³⁹⁾ v2.6.1 and quantified using featureCounts⁽⁴⁰⁾ v1.6.3. Read quality was assessed using FASTQC⁽⁴¹⁾ v0.11.7. All downstream analysis was performed in R⁽⁴²) v3.6.1. Differential expression analysis was performed via DESeq2⁽⁴³⁾ v1.24. RNA-seq data derived from whole blood RNA-seq in SARS-CoV-2 patients are deposited in the Gene Expression Omnibus (GEO) under the accession number GSE190413.

Differentially expressed lncRNA within both data-sets were identified using the lncRNA biotype-annotation within the Ensembl gene annotation system⁽⁴⁴⁾. A list of 389 interferon-stimulated genes (ISGs) was derived from published work⁽⁴⁵⁾. StringDB⁽⁴⁶⁾ and Cytoscape⁽⁴⁷⁾ were used in conjunction to generate an organically-clustered interactome of functionally-associated genes (StringDB, confidence>0.4). Edges represent a combination of significant ISG×ISG Pearson correlation and documented functional relationship as identified by StringDB (true for both criteria). Edge color represents the status of the ISG×ISG association following correction for CHROMR expression via partial correlation analysis.

Cell culture. CHROMR expression in human monocyte-derived macrophages infected with influenza A/California/04/09 (H1N1), influenza A/Wyoming/03/03 (H3N2), influenza A/Vietnam/1203/04 (H5N1) or mock-infected was examined by querying publicly available dataset GSE97672⁽²³⁾.

HEK293T and THP-1 cell lines were obtained from ATCC and the NF-κB-SEAP and IRF-Lucia luciferase Reporter Monocytes (THP-1-Dual cells) were obtained from InvivoGen. All cell lines were authenticated using standard ATCC methods (morphology check by microscope, growth curve analysis) and tested monthly for mycoplasma contamination. HEK293T were maintained in high-glucose DMEM (Corning) supplemented with 10% fetal bovine serum (FBS, Life Technologies) and 1% penicillin/streptomycin (P/S, Life Technologies). THP-1 cells were maintained in RPMI 1640 (ATCC) supplemented with 10% FBS and 1% P/S. THP-1-Dual cells were maintained in RPMI 1640 supplemented 10% FBS, 1% P/S, and 50 μg/mL of Normocin (InvivoGen). THP-1-Dual cells were cultured with selectable marker Zeocin (100 μg/mL, InvivoGen) every other passage to maintain stable integration of inducible reporter constructs. THP-1 cells and THP-1-Dual Cells were differentiated into macrophages in the presence of 100 nM phorbol-12-myristate acetate (PMA, Sigma) for 48-72 h.

Transient knockdown of CHROMR was acquired as follows; PMA-differentiated THP-1 cells or PMA-differentiated THP-1-Dual cells were transfected with 62.5 nM locked nucleic acid GapmeRs (Qiagen) targeting a common region of all CHROMR variants (GapCHROMR) or Negative Control A (GapCTRL) using Lipofectamine RNAiMax (Life Technologies) as described⁽²⁰⁾. CHROMR3-overexpressing THP-1 cells were created as described⁽²⁰⁾, and cultured under selection pressure puromycin (5 μg/mL, Thermo Fisher Scientific) to maintain purity. Knockdown of IRF2 and IRF2BP2 was acquired by transfecting 100 nM siRNA directed against IRF2 (Qiagen, GS3660) or IRF2BP2 (Qiagen, GS359948) using Lipofectamine RNAiMax into THP-1 macrophages, Allstars Negative Control (Qiagen, 1027280) was used control.

RNA isolation, cell fractionation and qPCR. Total RNA was isolated using TRIzol reagent (Invitrogen) and Direct-zol RNA MicroPrep columns (Zymo Research). For cell fractionation experiments RNA was isolated from separate cytoplasmic and nuclear fractions using the PARIS kit (Thermo Fisher Scientific). Upon isolation, RNA was reverse transcribed using iScript cDNA Synthesis kit (Bio-Rad Laboratories) and quantitative PCR analysis was conducted using KAPA SYBR green Supermix (KAPA Biosystems) according to the manufacturer's instructions and quantified on Quantstudio 3 (Applied Biosystems). Fold change in mRNA expression was calculated using the comparative cycle method (2^{-ΔΔCt}) normalized to the housekeeping gene GAPDH. A list of primers used in this study can be found in Extended Data Table 6.

Cellular response to microbial ligands and influenza infection. To assess the response of macrophages to TLR-activation and viral infection, we stimulated THP-1 macrophages or GapmeR-treated THP-1 macrophages with either 100-500 ng/ml lipopolysaccharide (LPS, Invivogen), 1 μg/mL polyinosinic: polycytidylic acid (poly(I:C), Invivogen), influenza A virus/WSN/1933 (H1N1) or vehicle control for indicated time periods. After treatment RNA was isolated and analyzed. Supernatants of GapCTRL- and GapCHROMR-treated THP-1 stimulated 24 h with 1 μg/mL of poly(I:C) was collected to measure accumulated levels of secreted cytokines. Levels of cytokines in supernatants were quantified using LEGENDplex Human Proinflammatory Cytokine Panel 1 (BioLegend, 740985) according to manufacturers' instructions.

RNA-sequencing. RNA was isolated from THP-1 macrophages treated with CHROMR-targeting GapmeRs or negative control and subsequently stimulated with 500 ng/mL LPS or 1 μg/mL poly(I:C) (InvivoGen) for indicated times. RNA was used to generate barcoded cDNA libraries using the TruSeq RNA Sample Preparation kit (Illumina). Indexed libraries were pooled and sequenced (paired-end 50 or 100 bp reads) on the Illumina HiSEQ platform. RNA-seq reads were aligned using the STAR Aligner against hg38 annotations. Gene counting was done using featureCounts. Raw counts were normalized and DE-analysis was performed using DESeq2. Ingenuity Pathway Analysis (Qiagen) was used to evaluate the most significantly altered pathways and upstream regulators. RNA-seq data are deposited in the GEO under the accession number GSE190413.

Gene expression profiling. RNA was isolated from THP-1 macrophages stable overexpressing CHROMR or an empty vector control, reverse-transcribed, and qPCR analysis of Type I Interferon Response genes was performed using RT² Profiler PCR Arrays (Qiagen, PAHS-016ZA) according to manufacturer's protocol. Data analysis was performed using the manufacturer's integrated web-based software package of the PCR Array System using ΔΔCt-based fold change calculations.

Quantification of influenza A virus infection in THP-1 macrophages. THP-1 macrophages transiently knocked down for CHROMR were infected with 100, 500 or 1000 plaque forming units (PFU, as determined on MDCK cells) of influenza A/WSN/1933 (H1N1) virus. The virus inoculate was diluted in DPBS supplemented with calcium and magnesium. Cell growth media was replaced by virus dilution and incubated for 1 h at 37° C. and 5% CO₂. After 1 h, the virus was aspirated, RPMI 1640 with 20% FBS was added to the cells, and cells were incubated at 37° C. and 5% CO₂. At 24 h, the cells were fixed with 8% paraformaldehyde (Thermo Fisher Scientific), quenched with 50 mM NH₄Cl and washed with PBS. Cells were stained with a monoclonal mouse anti-NP antibody (Sigma, MAB8251) followed by anti-mouse Alexa 488 secondary antibody (Thermo Fisher Scientific, R37120) and nuclear staining (4′,6-diamidino-2-phenylindole (DAPI, Sigma). Cells were washed with PBS leaving the last wash on before imaging. Plates were imaged using the Cell-Insight CX7 high-content screening platform. Images were analyzed and quantified with HCS Navigator software for total and infected cell numbers.

THP-1-Dual reporter assay. THP-1-Dual cells were differentiated towards macrophages using PMA and subsequently transfected with GapmeRs targeted CHROMR or a GapmeR control as described above, 24 h post-transfection the THP-1-Dual cells were treated with 1 μg/mL poly(I:C). Supernatants were taken on indicated time points and activation of NF-κB was measured by detecting secreted alkaline phosphatase (SEAP) using Quanti-Blue (InvivoGen); activation of the Interferon Regulatory Factor (IRF) at the ISRE was measured by detecting luciferase levels in the supernatants using Quanti-Luc (InvivoGen). Detected levels of SEAP and luciferase at the start of the experiment (0 h) were set to 100%.

Chromatin immunoprecipitation. GapCTRL and GapCHROMR treated THP-1 macrophages were cross-linked with 1% glutaraldehyde (Thermo Fisher Scientific) for 10 min at room temperature, and the cross-linking was quenched twice with 125 mM glycine/PBS for 5 min each. Cells were washed twice with ice-cold PBS, and harvested by scraping. Nuclear pellets were isolated by swelling cross-linked cells in hypotonic lysis buffer (25 mM HEPES pH 7.4, 1.5 mM MgCl₂, 10 mM KCl, 0.5% NP-40 and 1 mM DTT) supplemented with 1× HALT protease inhibitor cocktail (Promega) on ice for 15 min, followed by dounce homogenization. Nuclear pellets were suspended in sonication buffer (50 mM HEPES pH 7.4, 140 mM NaCl, 1 mM EDTA, 1% Triton-X 100, 0.1% sodium deoxycholate, 0.5% SDS, 1 mM DTT and 1× protease inhibitor cocktail) and incubated at ice for 10 min. Nuclear extracts were sonicated using Bioruptor UCD-200 (Diagenode Inc.) for 10×1 min cycles of “30 sec ON/OFF” at the highest voltage setting to generate 200-500 bp chromatin fragments. In each experiment, chromatin was first processed by agarose gel electrophoresis to confirm DNA shearing to 200-500 bp fragments, and the DNA concentration was measured by NanoDrop 2000. Equal quantities of sheared chromatin (10 μg per immunoprecipitation) were diluted 1:5 in sonication buffer to the final volume of 1 mL, and immunoprecipitated overnight with 1 μg antibody targeting human histone H3K27Ac (Active Motif, 39685) or isotype control IgG antibody (Sigma, 12-370) at 4° C. overnight. Chromatin complexes were captured using 20 μL Dynabeads protein G (Invitrogen) at 4° C. for 1 h. Beads were washed once with sonication buffer (containing 0.1% SDS), two times with high salt buffer (50 mM HEPES pH 7.4, 500 mM NaCl, 1 mM EDTA, 1% Triton-X 100, 0.1% sodium deoxycholate, 0.1% SDS), two times with LiCl buffer (20 mM Tris pH 7.4, 250 mM LiCl, 1 mM EDTA, 0.5% NP-40, 0.1% sodium deoxycholate, 0.05% Tween-20), and once with Tris-EDTA buffer (10 mM Tris pH 7.4, 1 mM EDTA). Each wash was performed at room temperature for 5 min in 1 mL volume. Beads were captured using DynaMag magnet (Thermo Fisher Scientific). Elution was performed by suspending beads in 100 μL elution buffer (20 mM Tris pH 7.4, 1% SDS, 50 mM NaHCO₃, 1 mM EDTA). Chromatin immunoprecipitation (ChIP) eluates were reverse cross-linked at 65° C. for 4 h, digested with proteinase K (Thermo Fisher Scientific, 10 μg/mL) at 55° C. for 1 h and 2 μL RNase cocktail (Ambion) at 37° C. for 30 min.

ChIP-sequencing. ChIP purified DNA was cleaned using PCR purification columns (Qiagen) and subjected to Illumina sequencing. Next, the overall quality of the sequenced ChIP-seq libraries was assessed with FastQC⁽⁴¹⁾. To remove contaminating sequencing adapters and low-quality bases, reads were trimmed using Fastp⁽⁴⁸⁾. FastQC was run again on the trimmed reads to analyze the global impact of trimming. Reads were then aligned to the human genome (hg38) using Bowtie2⁽⁴⁹⁾. Alignments were sorted and indexed using Samtools for downstream processes⁽⁵⁰⁾. MACS2 was then used to identify significant peaks⁽⁵¹⁾. Peaks with a Q-value of less than 0.05 were retained. Custom scripts and the ChIPQC R package were used to assess ChIP-seq peak quality and reproducibility⁽⁵²⁾. Peaks present in all replicates from each condition were retained for differential enrichment analysis. Peaks were annotated using ChIPseeker package from Bioconductor⁽⁵³⁾ Peaks that overlapped a 4 kb window centered at an annotated transcription start site were annotated as promoter peaks. Differential enrichment analysis was performed using DiffBind package from Bioconductor⁽⁵⁴⁾. Peaks with a false discovery rate (FDR-)adjusted P-value of 0.1 or less were considered differentially enriched between the knockdown and control conditions. Functional analysis of significantly enriched peaks was performed using Genomic Regions Enrichment Annotations Tool (GREAT) with default parameters⁽⁵⁵⁾. Motif enrichment analysis was performed on 3 kb windows centered at the TSS of genes with differentially enriched promoter peaks using Hypergeometric Optimization of Motif EnRichment (HOMER) with the command ‘findMotifs.pl’⁽⁵⁶⁾. ChIP-seq data are deposited in the GEO under the accession number GSE190413.

Chromatin Isolation by RNA Precipitation (ChIRP). Cell harvesting, lysis, disruption, and chromatin isolation by RNA purification were performed as previously described⁽⁵⁷⁾ with the following modifications: (1) Cells were cross-linked in 3% formaldehyde for 30 min, followed by 0.125 M glycine quenching for 5 min; (2) Hybridization was performed for 16 h; (3) For mass spectrometry (MS) experiments, lysates were pre-cleared by incubating with 30 mL washed beads per mL of lysate at 37° C. for 30 min with mixing; (4) As a negative control, lysates were pooled and aliquoted into equal amounts and RNA was removed by incubating with RNase A (1 μg/mL, Sigma), and subsequent incubation at 37° C. for 30 min prior to hybridization steps. RNA, DNA, protein isolation was performed as described⁽⁵⁷⁾ and further detailed below for ChIRP followed by DNA-seq (ChIRP-seq) or Comprehensive Identification of RNA-binding Proteins by Mass Spectrometry (ChIRP-MS). RNA extraction was performed for validation of lncRNA enrichment. A list of probes used in this study can be found in Extended Data Table 6.

ChIRP followed by DNA-seq (ChIRP-seq). DNA was eluted from hybridized magnetic beads and subjected for Illumina sequencing. In short, beads were washed at room temperature with ChIRP wash buffer (EMD Millipore, #17-10494). Beads were subsequently captured using a DynaMag magnet (Thermo Fisher Scientific) and DNA was eluted by suspending beads in elution buffer (20 mM Tris pH 7.4, 1% SDS, 50 mM NaHCO3, 1 mM EDTA). ChIRP eluates were reverse cross-linked at 65° C. for 4 h, digested with Proteinase K (EMD Millipore) at 55° C. followed by incubation with RNase cocktail (Ambion). ChIRP purified DNA was cleaned using PCR purification columns (Zymo Research) and subjected to Illumina sequencing. Reads were trimmed using Trimmomatic⁽⁵⁸⁾ and mapped to hg19 using BWA⁽⁵⁹⁾. Peaks were then called for each probe set and replicate using the ‘callpeak’ function from MACS2⁽⁵¹⁾ relative to the input from the same replicate. Peaks were then imported into the DiffBind package from Bioconductor⁽⁵⁴⁾ and differential peaks were called between even and odd probe sets. Only peaks with no differential binding between the probe sets were retained. Peaks were then assigned to their nearest genomic location using ChIPseeker package from Bioconductor⁽⁵³⁾. ChIRP-seq data are deposited in the GEO under the accession number GSE190413

Comprehensive Identification of RNA-binding Proteins by Mass Spectrometry (ChIRP-MS). Protein was isolated from magnetic beads and analyzed by MS. To elute protein beads were collected on magnetic stand, resuspended in biotin elution buffer (12.5 mM D-biotin (Thermo Fisher Scientific), 7.5 mM HEPES pH 7.5, 75 mM NaCl, 1.5 mM EDTA, 0.15% SDS, 0.075% sarkosyl, and 0.02% sodium deoxycholate). Trichloroacetic acid (25% of total volume) was added to the clean eluent and proteins were precipitated at 4° C. overnight. Proteins were pelleted at 16,000 g at 4° C. for 30 min, washed with cold acetone and pelleted again at 16,000 g at 4° C. for 5 min. Proteins were immediately solubilized in desired volumes of Laemmli sample buffer (Invitrogen) and boiled at 95° C. for 30 min with occasional mixing to reverse crosslinking. Final protein samples were size-separated in Bis-Tris SDS-PAGE gels (Invitrogen) and submitted for MS analysis by the Proteomics Laboratory at NYU Langone Health. Individual samples were subjected to liquid chromatography (LC) separation with MS using the autosampler of an EASY-nLC 1000 (Thermo Fisher Scientific). Subsequently, peptides were gradient eluted from the column directly to Q Exactive mass spectrometer using a 1 h gradient (Thermo Fisher Scientific). High resolution full MS spectra were acquired with a resolution of 70,000, an AGC target of 1×10⁶, with a maximum ion time of 120 ms, and scan range of 400 to 1,500 m/z. Following each full MS twenty data-dependent high resolution HCD MS/MS spectra were acquired. All MS/MS spectra were collected using the following instrument parameters: resolution of 17,500, AGC target of 5×10⁴, maximum ion time of 120 ms, one microscan, 2 m/z isolation window, fixed first mass of 150 m/z, and NCE of 27. MS/MS spectra were searched against a UniProt human database, using Sequest⁽⁶⁰⁾ within Proteome Discoverer (Thermo Fisher Scientific). Only high confidence peptides, based on a better than 1% FDR searched against a decoy database, were included for peptide identification. Each protein was scored by the sum of the scores of the individual peptide sequences present. Mean score of three experiments is calculated as the average of the individual protein scores of each individual MS-experiment.

RNA Immunoprecipitation. Human histone H3, IRF2BP2, and HNRPNLL were immunoprecipitated from PMA-differentiated THP-1 macrophages. All immunoprecipitations were done using the MagnaRIP RNA-Binding Protein Immunoprecipitation Kit (EMD Millipore) according to the manufacturers' instructions. Briefly, an antibody targeting human histone H3 (Abcam, ab1791), IRF2BP2 (Abcam, ab220155), HNRNPLL (Cell Signaling, 4783), or an isotype matched control antibody (Sigma, 12-370 or 12-371) were bound to magnetic beads and incubated with lysed cells at 4° C. for 24 h. Beads were isolated and cleaved from the bound proteins by proteinase K, and coprecipitated RNA was purified. qPCR analysis of total RNA was performed to detect enrichment of CHROMR variants and control genes in the protein-of-interest precipitated fraction was determined as percentage of 1% input control.

RNA Fluorescence In Situ Hybridization. Custom Stellaris® FISH Probes were designed against CHROMR utilizing the Stellaris® FISH Probe Designer (LGC Biosearch Technologies). Formaldehyde-fixed THP-1 macrophages were permeabilized with 70% isopropanol and subsequently simultaneously hybridized with the CHROMR Stellaris® FISH Probe set labeled with Quasar® 670 Dye (LGC Biosearch Technologies) and a rabbit polyclonal antibody against IRF2BP2 (Atlas Antibodies, HPA062269), following the manufacturer's protocol. IRF2BP2 was visualized using fluorescent goat anti-rabbit secondary antibodies (Thermo Fisher Scientific, A-21206) and DAPI was used to visualize nuclear DNA.

Mutagenesis studies. The interaction between IRF2BP2 and CHROMR3 was analysed by mutating the putative interaction site between IRF2BP2 and CHROMR3. Two GG-doublets in the sequence of a plasmid overexpressing CHROMR3⁽²⁰⁾ were replaced with two CC-doublets creating a plasmid overexpressing CHROMR3-G4mut. All mutations were performed using the Quickchange XL kit (Stratagene) using the primers indicated in Extended Data Table 6. CHROMR3-G4mut expression plasmids were confirmed by Sanger sequencing. Next, human IRF2BP2, CHROMR3 and CHROMR3-G4mut were overexpressed in HEK293T cells using plasmids overexpressing a MYC/DDK tagged IRF2BP2 (OriGene Technologies, RC213250) and plasmids overexpressing CHROMR3 and CHROMR3-G4mut using Lipofectamine 2000 (Thermo Fisher Scientific). Antibodies directed against MYC/DDK (OriGene Technologies, TA50011) or an isotype matched control antibody (Sigma, 12-370) were used in immunoprecipitations as described above.

Bioinformatics. Enrichment analysis of interferon-stimulated genes was performed using Enrichr⁽²⁵⁾, the web-based software for Gene Set Enrichment Analysis was used for ChIP Enrichment Analysis (ChEA)⁽⁶¹⁾ database (2016) which contains results from transcription factor ChIP-seq studies extracted from supporting material. Results were manually curated to remove duplicate studies or studies performed with non-human transcription factors. The catRAPID algorithm⁽⁶²⁾ was used to estimate the binding propensity of IRF2BP2 and CHROMR. The interaction score is generated using the interaction propensity distribution of a reference set, as described⁽⁶³⁾ The QGRS Mapper⁽³⁴⁾ was used for recognition and mapping of putative quadruplexes in CHROMR. RNAfold, part of The Vienna RNA Websuite⁽⁶⁴⁾, was used to predict the minimum free energy secondary structure of CHROMR3 and the RNA plot was created with RNArtist, developed by Fabrice Jossinet and available at github.com/fjossinet/RNArtist.

Statistics. Statistical significance between two groups of independent biological replicates was evaluated with Student's t-test. One-way ANOVA was performed when comparing three groups or more for one variable (univariate comparisons), followed if significant by either Dunnett's post-hoc multiple comparisons test (MCT) was used when comparing to a control group, or Sidak's post-hoc MCT was used when comparing pre-selected groups. For non-parametric measurements, Kruskal-Wallis test was performed followed by Dunn's MCT. Repeated-measure two-way ANOVA was used when comparing two groups or more for bivariate analyses followed by Sidak's post-hoc MCT to compare groups if either group or group×time interaction was significant.

Pearson correlation was used to examine ISG×ISG correlation in influenza A infected patients, for which the sample size of n=41 can detect a correlation of r=0.43 with α=0.05 and a power of 80%. Robust third-order polynomial non-linear regression was used to assess distribution of lncRNA×ISG correlation coefficient in function of differential expression in influenza A infection to minimize outlier impact. RNA-seq normalized transcript data were log_{1P}-transformed to normalize distribution for partial correlation analysis. Partial correlation analysis was used to control for CHROMR expression as a covariate within ISG×ISG associations. Pearson and partial correlation coefficients were compared by Fisher-r-to-Z transformation followed by Z-test.

Statistical significance of enrichment in top canonical pathways and top upstream regulators (cytokines and transcriptional regulators) is calculated in Ingenuity Pathway Analysis by a right-tailed Fisher's exact test. Enrichr, GREAT and HOMER use a binomial test to calculate significant enrichment in biological process or motif enrichment, respectively. Statistical analyses were performed using GraphPad Prism software, bivariate and partial correlation analyses were performed in R studio. Threshold for statistical significance was P≤0.05. All quantitative data are presented as mean±standard error of the mean (sem).

### Example 5

### ASOs Against the G-Quadruplex Sequence of CHROMR Decrease ISG Expression

We showed that upregulation of CHROMR leads to sequestering of IRF2BP2/IRF2 repressor complex, and licensing of IRF-dependent transcriptional activation of the ISG network. While this is protective against viral infection, the ability of CHROMR to sequester an endogenous repressor of the ISG network expression could contribute, at least in part, to the elusive mechanism of increased type I IFN signature in SLE, the prototypical type I inteferonopathy. In support of this, CHROMR expression was strongly correlated with the expression of a subset of 27 ISG selected from an IFN module scoring classification that can be used to differentiate SLE patients from healthy controls⁽⁶⁶⁾ (FIG. 10a). In addition, a preliminary analysis within a subset of patients from a publicly available dataset⁽⁶⁷⁾ suggested a potential correlation of plasma CHROMR levels with autoantibody status, suggesting a relationship between CHROMR and immunoregulatory mechanisms within SLE (FIG. 10b). To test whether targeting CHROMR would be relevant in the treatment of SLE, we performed experiments in human THP1-Dual macrophages, an ISG reporter cell line that expresses Luciferase downstream of IFN-stimulated response elements (ISRE). We showed that downregulation of CHROMR using a GapmeR ASO (GapCHROMR, ASO_Gap/3) reduces ISRE-driven reporter expression in response to the SLE-relevant agonist imiquimod compared to GapmeR control (GapCTL).

To identify a precise sequence that required to be targeted to interfere with the interaction between CHROMR and IRF2BP2 and negate ISG production, we designed short CHROMR-targeting ASOs of 7 to 13 nucleotides (7-Mer, 13-Mer) that can hybridize with the four critical GG pairs of the G4 structure, but cannot be recognized by TLR and do not recruit RNAseH (FIG. 11c, FIG. 12c)⁽⁶⁸⁾. Using IFNβ to induce a robust IFN response in THP1-Dual reporter cells, we assessed the ability of each of the CHROMR-targeting ASOs to inhibit ISG expression relative to a non-targeting control of equal length. ASO_7/1, designed against the first GG-pair, was able to decrease ISG expression in the presence of IFNβ (FIG. 11a). Neither ASOs designed against the neighboring GG-pairs, nor a non-targeting control ASO had any effect on ISG expression. The use of ASO_7/1 did not induce changes in CHROMR transcript expression in the presence of absence of IFNβ, although IFNβ did induce an increase in CHROMR expression (FIG. 11b). Similarly, ASO_13/1 designed against the 1^{st} and 2^{nd} GG-pair was also efficient at reducing IFNβ-induced ISG expression, while ASOs against the neighboring GG-pairs were not (FIG. 12a). Treatment with ASO_13/1 also did not lead to a decrease in CHROMR expression (FIG. 12b). As a proof-of-concept, use of GapmeR sequence ASO_Gap/3 led to a decrease in IFNβ-induced ISG expression (FIG. 13a) and to a decrease in CHROMR expression (FIG. 13b), as intended. None of the ASOs induced ISG expression in absence of an inflammatory stimulus (Vehicle; FIG. 11a, 12a, 13a).

To confirm that only specific ASOs interfere with the binding of CHROMR to IRF2BP2, we performed an RNA-immunoprecipitation in human embryonic kidney (HEK293T) cell (FIG. 14a). First, we showed that IRF2BP2 IP led to a 30-fold enrichment of CHROMR after overexpression relative to a control empty vector (EV; FIG. 14b). Next, we showed that transfection of CHROMR-expressing HEK293T cells with ASO_7/1 (FIG. 14c-d) or ASO_13/1 (FIGS. 14e-f) reduced the binding of CHROMR to IRF2BP2 by ~75% compared to a non-targeting control ASOs.

To test how ASOs targeting CHROMR's G-quadruplex or expression level alter inflammation and immune cell interactions in human tissue, we used a 3D organ-derived human vascular explant model amenable to short term molecular studies ex vivo (96h; FIG. 15a). To demonstrate the role of lncRNA CHROMR in licensing inflammation, we transfected freshly isolated atherosclerotic carotid explants (3 mm²) obtained from subjects undergoing carotid endarterectomy with a CHROMR overexpression vector. CHROMR overexpression resulted in significant production of pro-inflammatory cytokines compared to a control empty overexpression vector (FIG. 15b). To test the effect of a reduction in CHROMR expression on inflammation, ASO_Gap/3 was used in the presence of TLR-3 agonist poly(I:C) to induce a type I IFN response, which resulted in a significant reduction in proinflammatory cytokine production compared to its non-targeting control ASOs (FIG. 15c). CHROMR's G-quadruplex-targeting ASO_7/1 had a similar effect under identical conditions (FIG. 15d).

These data demonstrate the necessity of either targeting the first GG-pair of CHROMR's G-quadruplex, or decreasing CHROMR expression, to efficiently reduce type I IFN-induced ISG expression. This effect cannot be observed when targeting other GG-pairs present in CHROMR's G-quadruplex and may be attributed to decreased binding of CHROMR to IRF2BP2.

### REFERENCES—THIS REFERENCE LISTING IS NOT AN INDICATION THAT ANY REFERENCE IS MATERIAL TO PATENTABILITY

1. W. M. Schneider, M. D. Chevillotte, C. M. Rice, Interferon-stimulated genes: a complex web of host defenses. Annu Rev Immunol 32, 513-545 (2014).
2. A. Michalska, K. Blaszczyk, J. Wesoly, H. A. R. Bluyssen, A Positive Feedback Amplifier Circuit That Regulates Interferon (IFN)-Stimulated Gene Expression and Controls Type I and Type II IFN Responses. Front Immunol 9, 1135 (2018).
3. H. Feng, Y. B. Zhang, J. F. Gui, S. M. Lemon, D. Yamane, Interferon regulatory factor 1 (IRF1) and anti-pathogen innate immune responses. PLoS Pathog 17, e1009220 (2021).
4. Y. J. Crow et al., Cree encephalitis is allelic with Aicardi-Goutieres syndrome: implications for the pathogenesis of disorders of interferon alpha metabolism. J Med Genet 40, 183-187 (2003).
5. T. Derrien et al., The GENCODE v7 catalog of human long noncoding RNAs: analysis of their gene structure, evolution, and expression. Genome Res 22, 1775-1789 (2012).
6. J. Harrow et al., GENCODE: the reference human genome annotation for The ENCODE Project. Genome Res 22, 1760-1774 (2012).
7. P. P. Amaral, J. S. Mattick, Noncoding RNA in development. Mamm Genome 19, 454-492 (2008).
8. J. L. Rinn, H. Y. Chang, Genome regulation by long noncoding RNAs. Annu Rev Biochem 81, 145-166 (2012).
9. J. Fan, M. Cheng, X. Chi, X. Liu, W. Yang, A Human Long Non-coding RNA LncATV Promotes Virus Replication Through Restricting RIG-I-Mediated Innate Immunity. Front Immunol 10, 1711 (2019).
10. M. Jiang et al., Self-Recognition of an Inducible Host lncRNA by RIG-I Feedback Restricts Innate Immune Response. Cell 173, 906-919 e913 (2018).
11. H. Lin et al., The long noncoding RNA Lnczc3h7a promotes a TRIM25-mediated RIG-I antiviral innate immune response. Nat Immunol 20, 812-823 (2019).
12. Q. Xie et al., Long Noncoding RNA ITPRIP-1 Positively Regulates the Innate Immune Response through Promotion of Oligomerization and Activation of MDA5. J Virol 92 (2018).
13. X. Li et al., Long Noncoding RNA Lnc-MxA Inhibits Beta Interferon Transcription by Forming RNA-DNA Triplexes at Its Promoter. J Virol 93 (2019).
14. S. Agarwal et al., The long non-coding RNA LUCAT1 is a negative feedback regulator of interferon responses in humans. Nat Commun 11, 6348 (2020).
15. M. Barriocanal, E. Carnero, V. Segura, P. Fortes, Long Non-Coding RNA BST2/BISPR is Induced by IFN and Regulates the Expression of the Antiviral Factor Tetherin. Front Immunol 5, 655 (2014).
16. S. Kulkarni et al., CCR5AS lncRNA variation differentially regulates CCR5, influencing HIV disease outcome. Nat Immunol 20, 824-834 (2019).
17. H. Kambara et al., Negative regulation of the interferon response by an interferon-induced long non-coding RNA. Nucleic Acids Res 42, 10668-10680 (2014).
18. J. Ouyang et al., NRAV, a long noncoding RNA, modulates antiviral responses through suppression of interferon-stimulated gene transcription. Cell Host Microbe 16, 616-626 (2014).
19. B. Mariotti et al., The Long Non-coding RNA NRIR Drives IFN-Response in Monocytes: Implication for Systemic Sclerosis. Front Immunol 10, 100 (2019).
20. E. J. Hennessy et al., The long noncoding RNA CHROME regulates cholesterol homeostasis in primate. Nat Metab 1, 98-110 (2019).
21. E. L. Tsalik et al., The Host Response to Viral Infections Reveals Common and Virus-Specific Signatures in the Peripheral Blood. Front Immunol 12, 741837 (2021).
22. W. Liu et al., LncRNA Malat1 inhibition of TDP43 cleavage suppresses IRF3-initiated antiviral innate immunity. Proc Natl Acad Sci USA 117, 23695-23706 (2020).
23. S. Heinz et al., Transcription Elongation Can Affect Genome 3D Structure. Cell 174, 1522-1536 e1522 (2018).
24. J. N. Hutchinson et al., A screen for nuclear transcripts identifies two linked noncoding RNAs associated with SC35 splicing domains. BMC Genomics 8, 39 (2007).
25. M. V. Kuleshov et al., Enrichr: a comprehensive gene set enrichment analysis web server 2016 update. Nucleic Acids Res 44, W90-97 (2016).
26. D. Panda et al., IRF1 Maintains Optimal Constitutive Expression of Antiviral Genes and Regulates the Early Antiviral Response. Front Immunol 10, 1019 (2019).
27. B. Wang, P. Tontonoz, Liver X receptors in lipid signalling and membrane homeostasis. Nat Rev Endocrinol 14, 452-463 (2018).
28. C. Chu, H. Y. Chang, ChIRP-MS: RNA-Directed Proteomic Discovery. Methods Mol Biol 1861, 37-45 (2018).
29. P. Luthra et al., Topoisomerase II Inhibitors Induce DNA Damage-Dependent Interferon Responses Circumventing Ebola Virus Immune Evasion. mBio 8 (2017).
30. K. S. Childs, S. Goodbourn, Identification of novel co-repressor molecules for Interferon Regulatory Factor-2. Nucleic Acids Res 31, 3016-3026 (2003).
31. K. I. Masood et al., Upregulated type I interferon responses in asymptomatic COVID-19 infection are associated with improved clinical outcome. Sci Rep 11, 22958 (2021).
32. R. Ramalho-Oliveira, B. Oliveira-Vieira, J. P. B. Viola, IRF2BP2: A new player in the regulation of cell homeostasis. J Leukoc Biol 106, 717-723 (2019).
33. M. Bellucci, F. Agostini, M. Masin, G. G. Tartaglia, Predicting protein associations with long noncoding RNAs. Nat Methods 8, 444-445 (2011).
34. O. Kikin, L. D'Antonio, P. S. Bagga, QGRS Mapper: a web-based server for predicting G-quadruplexes in nucleotide sequences. Nucleic Acids Res 34, W676-682 (2006).
35. D. Varshney, J. Spiegel, K. Zyner, D. Tannahill, S. Balasubramanian, The regulation and functions of DNA and RNA G-quadruplexes. Nat Rev Mol Cell Biol 21, 459-474 (2020).
36. S. Bajimaya, T. Frankl, T. Hayashi, T. Takimoto, Cholesterol is required for stability and infectivity of influenza A and respiratory syncytial viruses. Virology 510, 234-241 (2017).
37. D. W. Sanders et al., SARS-CoV-2 requires cholesterol for viral entry and pathological syncytia formation. Elife 10 (2021).
38. https://igordot.github.io/sns/(2020) Igor Pro.
39. A. Dobin et al., STAR: ultrafast universal RNA-seq aligner. Bioinformatics (Oxford, England) 29, 15-21 (2013).
40. Y. Liao, G. K. Smyth, W. Shi, featureCounts: an efficient general purpose program for assigning sequence reads to genomic features. Bioinformatics (Oxford, England) 30, 923-930 (2014).
41. S. Andrews, FastQC A Quality Control tool for High Throughput Sequence Data. (2008).
42. R. Core Team, R: A Language and Environment for Statistical Computing (R Foundation for Statistical Computing, Vienna, Austria, 2018).
43. M. I. Love, W. Huber, S. Anders, Moderated estimation of fold change and dispersion for RNA-seq data with DESeq2. Genome Biology 15, 550 (2014).
44. B. L. Aken et al., The Ensembl gene annotation system. Database (Oxford) 2016 (2016).
45. J. W. Schoggins et al., A diverse range of gene products are effectors of the type I interferon antiviral response. Nature 472, 481-485 (2011).
46. D. Szklarczyk et al., STRING v10: protein-protein interaction networks, integrated over the tree of life. Nucleic Acids Res 43, D447-452 (2015).
47. P. Shannon et al., Cytoscape: a software environment for integrated models of biomolecular interaction networks. Genome Res 13, 2498-2504 (2003).
48. S. Chen, Y. Zhou, Y. Chen, J. Gu, fastp: an ultra-fast all-in-one FASTQ preprocessor. Bioinformatics 34, 1884-1890 (2018).
49. B. Langmead, S. L. Salzberg, Fast gapped-read alignment with Bowtie 2. Nat Methods 9, 357-359 (2012).
50. H. Li et al., The Sequence Alignment/Map format and SAMtools. Bioinformatics 25, 2078-2079 (2009).
51. Y. Zhang et al., Model-based analysis of ChIP-Seq (MACS). Genome Biol 9, R137 (2008).
52. T. S. Carroll, Z. Liang, R. Salama, R. Stark, I. de Santiago, Impact of artifact removal on ChIP quality metrics in ChIP-seq and ChIP-exo data. Front Genet 5, 75 (2014).
53. G. Yu, L. G. Wang, Q. Y. He, ChIPseeker: an R/Bioconductor package for ChIP peak annotation, comparison and visualization. Bioinformatics 31, 2382-2383 (2015).
54. R. B. Stark, G. D. (2011) DiffBind: Differential Binding Analysis of ChIP-Seq Peak Data. Bioconductor.
55. C. Y. McLean et al., GREAT improves functional interpretation of cis-regulatory regions. Nat Biotechnol 28, 495-501 (2010).
56. S. Heinz et al., Simple combinations of lineage-determining transcription factors prime cis-regulatory elements required for macrophage and B cell identities. Mol Cell 38, 576-589 (2010).
57. C. Chu, J. Quinn, H. Y. Chang, Chromatin isolation by RNA purification (ChIRP). J Vis Exp 10.3791/3912 (2012).
58. A. M. Bolger, M. Lohse, B. Usadel, Trimmomatic: a flexible trimmer for Illumina sequence data. Bioinformatics 30, 2114-2120 (2014).
59. H. Li, R. Durbin, Fast and accurate short read alignment with Burrows-Wheeler transform. Bioinformatics 25, 1754-1760 (2009).
60. J. K. Eng, A. L. McCormack, J. R. Yates, An approach to correlate tandem mass spectral data of peptides with amino acid sequences in a protein database. J Am Soc Mass Spectrom 5, 976-989 (1994).
61. A. Lachmann et al., ChEA: transcription factor regulation inferred from integrating genome-wide ChIP-X experiments. Bioinformatics 26, 2438-2444 (2010).
62. A. Armaos, A. Colantoni, G. Proietti, J. Rupert, G. G. Tartaglia, catRAPID omics v2.0: going deeper and wider in the prediction of protein-RNA interactions. Nucleic Acids Res 49, W72-W79 (2021).
63. F. Agostini, D. Cirillo, B. Bolognesi, G. G. Tartaglia, X-inactivation: quantitative predictions of protein interactions in the Xist network. Nucleic Acids Res 41, e31 (2013).
64. A. R. Gruber, R. Lorenz, S. H. Bernhart, R. Neubock, I. L. Hofacker, The Vienna RNA website. Nucleic Acids Res 36, W70-74 (2008).
66. Y. M. El-Sherbiny et al., A novel two-score system for interferon status segregates autoimmune diseases and correlates with clinical features. Sci Rep 8, 5793 (2018).
67. R. Rai, S. K. Chauhan, V. V. Singh, M. Rai, G. Rai, RNA-seq Analysis Reveals Unique Transcriptome Signatures in Systemic Lupus Erythematosus Patients with Distinct Autoantibody Specificities. PLoS One 11, e0166312 (2016).
68. M. Winkle, S. M. El-Daly, M. Fabbri, G. A. Calin, Noncoding RNA therapeutics-challenges and potential solutions. Nat Rev Drug Discov 20, 629-651 (2021).

TABLE 1
Shortest antisense-oligonucleotide
(ASO) sequences (DNA, 5′-3′)
that can be used to block CHROMR′s
interaction with IRF2BP2 without
causing CHROMR degradation (ASO_7_1)
and GapmeR ASO that can cause
CHROMR degradation (ASO_Gap_3).
ASO SEQUENCES TO BE PATENTED

| ID | DNA Sequence (5′-3′) | Mechanism of action |
|---|---|---|
| ASO 7/1 | CCCCCAT | Prevents CHROMR |
| | | binding to IRF2BP2 |
| | | Decreases ISG |
| | | expression |
| ASO 13/1 | GGACCTCCCCCAT | Prevents CHROMR |
| | (SEQ ID NO: 17) | binding to IRF2BP2 |
| | | Decreases |
| | | ISG expression |
| ASO_Gap/3 | CTCATAAGAAAACTGA | Causes CHROMR |
| | (SEQ ID NO: 1) | degradation |
| | | Decreases ISG |
| | | expression |

TABLE 2
List of ASO sequences (DNA, 5′-3′) that have been tested for their
ability to inhibit interferon-stimulated gene expression.
The highlighted ASOs (bold) showed efficacy whether via
containing the 5′-CCCCCAT-3′ DNA sequence or by causing
degradation of CHROMR (ASO_Gap_3).
Abbreviations: Interferon-stimulated gene (ISG).
ASOS TESTED IN PROOF-OF-CONCEPT EXPERIMENT

| ID | DNA Sequence (5′-3′) | Mechanism of action |
|---|---|---|
| ASO 7/1 | CCCCCAT | Prevents CHROMR binding to |
| | | IRF2BP2 |
| | | Decreases ISG expression |
| ASO 7/2 | GGGACCT | No effect on ISG expression |
| ASO 7/3 | TATCCGC | No effect on ISG expression |
| ASO 7/4 | GCCAGGA | No effect on ISG expression |
| ASO 13/1 | GGACCTCCCCCAT | Prevents CHROMR binding to |
| | (SEQ ID NO: 17) | IRF2BP2 |
| | | Decreases ISG expression |
| ASO 13/2 | TGCCAGGATATCC | No effect on ISG expression |
| | (SEQ ID NO: 18) | |
| ASO_Gap/3 | CTCATAAGAAAACTGA | Causes CHROMR degradation |
| | (SEQ ID NO: 1) | Decreases ISG expression |

TABLE 3
List of all control antisense-
oligonucleotide (ASO) sequences used
in proof-of-concept experiments.
CONTROL ASO SEQUENCES

| ID | DNA Sequence (5′-3′) | Use |
|---|---|---|
| ASO_ | CCTGCGC | Control for |
| LacZ/7 | | 7mer ASO |
| ASO_ | TAATCCGAGCCAG | Control for |
| LacZ/13 | (SEQ ID NO: 19) | 13mer ASO |
| ASO_ | GCGGTCGGGATAGTTT | Control for |
| GapLacZ | (SEQ ID NO: 20) | GapmeR |

TABLE 4
List of all possible ASO sequences (DNA, 5′-3′) of 7 to 20
nucleotides in length and containing the 5′-CCCCCAT-3′
DNA sequence that have shown efficacy. These sequences were
designed to allow flexibility in designing flapping sequences
to enhance specificity. Sequences highlighted (bold)
were experimentally shown to be efficient in reducing
interferon-stimulated gene expression.
Abbreviations: nucleotides (nts)
ASOs CONTAINING ‘CCCCCAT’ (SEQ ID NO: 1)

| Length (nts) | ID | DNA Sequence (5′-3′) |
|---|---|---|
| 7 | ASO 7/1 | CCCCCAT |
| 8 | ASO 8/28 | TCCCCCAT |
| 8 | ASO 8/29 | CCCCCATT |
| 9 | ASO 9/27 | CTCCCCCAT |
| 9 | ASO 9/28 | TCCCCCATT |
| 9 | ASO 9/29 | CCCCCATTT |
| 10 | ASO 10/26 | CCTCCCCCAT (SEQ ID NO: 21) |
| 10 | ASO 10/27 | CTCCCCCATT (SEQ ID NO: 22) |
| 10 | ASO 10/28 | TCCCCCATTT (SEQ ID NO: 23) |
| 10 | ASO 10/29 | CCCCCATTTG (SEQ ID NO: 24) |
| 11 | ASO_11/25 | ACCTCCCCCAT (SEQ ID NO: 25) |
| 11 | ASO 11/26 | CCTCCCCCATT (SEQ ID NO: 26) |
| 11 | ASO 11/27 | CTCCCCCATTT (SEQ ID NO: 27) |
| 11 | ASO 11/28 | TCCCCCATTTG (SEQ ID NO: 28) |
| 11 | ASO 11/29 | CCCCCATTTGA (SEQ ID NO: 29) |
| 12 | ASO 12/24 | GACCTCCCCCAT (SEQ ID NO: 30) |
| 12 | ASO 12/25 | ACCTCCCCCATT (SEQ ID NO: 31) |
| 12 | ASO 12/26 | CCTCCCCCATTT (SEQ ID NO: 32) |
| 12 | ASO 12/27 | CTCCCCCATTTG (SEQ ID NO: 33) |
| 12 | ASO 12/28 | TCCCCCATTTGA (SEQ ID NO: 34) |
| 12 | ASO 12/29 | CCCCCATTTGAG (SEQ ID NO: 35) |
| 13 | ASO 13/1 | GGACCTCCCCCAT (SEQ ID NO: 17) |
| 13 | ASO 13/23 | GACCTCCCCCATT (SEQ ID NO: 36) |
| 13 | ASO_13/24 | ACCTCCCCCATTT (SEQ ID NO: 37) |
| 13 | ASO 13/25 | CCTCCCCCATTTG (SEQ ID NO: 38) |
| 13 | ASO_13/26 | CTCCCCCATTTGA (SEQ ID NO: 39) |
| 13 | ASO 13/27 | TCCCCCATTTGAG (SEQ ID NO: 40) |
| 13 | ASO 13/28 | CCCCCATTTGAGA (SEQ ID NO: 41) |
| 14 | ASO 14/22 | GGGACCTCCCCCAT (SEQ ID NO: 42) |
| 14 | ASO 14/23 | GGACCTCCCCCATT (SEQ ID NO: 43) |
| 14 | ASO 14/24 | GACCTCCCCCATTT (SEQ ID NO: 44) |
| 14 | ASO 14/25 | ACCTCCCCCATTTG (SEQ ID NO: 45) |
| 14 | ASO 14/26 | CCTCCCCCATTTGA (SEQ ID NO: 46) |
| 14 | ASO_14/27 | CTCCCCCATTTGAG (SEQ ID NO: 47) |
| 14 | ASO 14/28 | TCCCCCATTTGAGA (SEQ ID NO: 48) |
| 14 | ASO 14/29 | CCCCCATTTGAGAA (SEQ ID NO: 49) |
| 15 | ASO 15/21 | TGGGACCTCCCCCAT (SEQ ID NO: 50) |
| 15 | ASO 15/22 | GGGACCTCCCCCATT (SEQ ID NO: 51) |
| 15 | ASO 15/23 | GGACCTCCCCCATTT (SEQ ID NO: 52) |
| 15 | ASO 15/24 | GACCTCCCCCATTTG (SEQ ID NO: 53) |
| 15 | ASO 15/25 | ACCTCCCCCATTTGA (SEQ ID NO: 54) |
| 15 | ASO 15/26 | CCTCCCCCATTTGAG (SEQ ID NO: 55) |
| 15 | ASO 15/27 | CTCCCCCATTTGAGA (SEQ ID NO: 56) |
| 15 | ASO 15/28 | TCCCCCATTTGAGAA (SEQ ID NO: 57) |
| 15 | ASO_15/29 | CCCCCATTTGAGAAG (SEQ ID NO: 58) |
| 16 | ASO 16/20 | TTGGGACCTCCCCCAT (SEQ ID NO: 59) |
| 16 | ASO 16/21 | TGGGACCTCCCCCATT (SEQ ID NO: 60) |
| 16 | ASO 16/22 | GGGACCTCCCCCATTT (SEQ ID NO: 61) |
| 16 | ASO 16/23 | GGACCTCCCCCATTTG (SEQ ID NO: 62) |
| 16 | ASO 16/24 | GACCTCCCCCATTTGA (SEQ ID NO: 63) |
| 16 | ASO 16/25 | ACCTCCCCCATTTGAG (SEQ ID NO: 64) |
| 16 | ASO 16/26 | CCTCCCCCATTTGAGA (SEQ ID NO: 65) |
| 16 | ASO 16/27 | CTCCCCCATTTGAGAA (SEQ ID NO: 66) |
| 16 | ASO 16/28 | TCCCCCATTTGAGAAG (SEQ ID NO: 67) |
| 16 | ASO 16/29 | CCCCCATTTGAGAAGG (SEQ ID NO: 68) |
| 17 | ASO_17/19 | TTTGGGACCTCCCCCAT (SEQ ID NO: 69) |
| 17 | ASO 17/20 | TTGGGACCTCCCCCATT (SEQ ID NO: 70) |
| 17 | ASO 17/21 | TGGGACCTCCCCCATTT (SEQ ID NO: 71) |
| 17 | ASO 17/22 | GGGACCTCCCCCATTTG (SEQ ID NO: 72) |
| 17 | ASO 17/23 | GGACCTCCCCCATTTGA (SEQ ID NO: 73) |
| 17 | ASO 17/24 | GACCTCCCCCATTTGAG (SEQ ID NO: 74) |
| 17 | ASO 17/25 | ACCTCCCCCATTTGAGA (SEQ ID NO: 75) |
| 17 | ASO_17/26 | CCTCCCCCATTTGAGAA (SEQ ID NO: 76) |
| 17 | ASO 17/27 | CTCCCCCATTTGAGAAG (SEQ ID NO: 77) |
| 17 | ASO 17/28 | TCCCCCATTTGAGAAGG (SEQ ID NO: 78) |
| 17 | ASO 17/29 | CCCCCATTTGAGAAGGG (SEQ ID NO: 79) |
| 18 | ASO 18/18 | CTTTGGGACCTCCCCCAT (SEQ ID NO: 80) |
| 18 | ASO 18/19 | TTTGGGACCTCCCCCATT (SEQ ID NO: 81) |
| 18 | ASO 18/20 | TTGGGACCTCCCCCATTT (SEQ ID NO: 82) |
| 18 | ASO 18/21 | TGGGACCTCCCCCATTTG (SEQ ID NO: 83) |
| 18 | ASO 18/22 | GGGACCTCCCCCATTTGA (SEQ ID NO: 84) |
| 18 | ASO 18/23 | GGACCTCCCCCATTTGAG (SEQ ID NO: 85) |
| 18 | ASO 18/24 | GACCTCCCCCATTTGAGA (SEQ ID NO: 86) |
| 18 | ASO 18/25 | ACCTCCCCCATTTGAGAA (SEQ ID NO: 87) |
| 18 | ASO 18/26 | CCTCCCCCATTTGAGAAG (SEQ ID NO: 88) |
| 18 | ASO 18/27 | CTCCCCCATTTGAGAAGG (SEQ ID NO: 89) |
| 18 | ASO 18/28 | TCCCCCATTTGAGAAGGG (SEQ ID NO: 90) |
| 18 | ASO 18/29 | CCCCCATTTGAGAAGGGC (SEQ ID NO: 91) |
| 19 | ASO 19/17 | GCTTTGGGACCTCCCCCAT (SEQ ID NO: 92) |
| 19 | ASO 19/18 | CTTTGGGACCTCCCCCATT (SEQ ID NO: 93) |
| 19 | ASO_19/19 | TTTGGGACCTCCCCCATTT (SEQ ID NO: 94) |
| 19 | ASO 19/20 | TTGGGACCTCCCCCATTTG (SEQ ID NO: 95) |
| 19 | ASO 19/21 | TGGGACCTCCCCCATTTGA (SEQ ID NO: 96) |
| 19 | ASO 19/22 | GGGACCTCCCCCATTTGAG (SEQ ID NO: 97) |
| 19 | ASO 19/23 | GGACCTCCCCCATTTGAGA (SEQ ID NO: 98) |
| 19 | ASO 19/24 | GACCTCCCCCATTTGAGAA (SEQ ID NO: 99) |
| 19 | ASO 19/25 | ACCTCCCCCATTTGAGAAG (SEQ ID NO: 100) |
| 19 | ASO 19/26 | CCTCCCCCATTTGAGAAGG (SEQ ID NO: 101) |
| 19 | ASO 19/27 | CTCCCCCATTTGAGAAGGG (SEQ ID NO: 102) |
| 19 | ASO 19/28 | TCCCCCATTTGAGAAGGGC (SEQ ID NO: 103) |
| 19 | ASO 19/29 | CCCCCATTTGAGAAGGGCA (SEQ ID NO: 104) |
| 20 | ASO 20/16 | CGCTTTGGGACCTCCCCCAT (SEQ ID NO: 105) |
| 20 | ASO 20/17 | GCTTTGGGACCTCCCCCATT (SEQ ID NO: 106) |
| 20 | ASO_20/18 | CTTTGGGACCTCCCCCATTT (SEQ ID NO: 107) |
| 20 | ASO_20/19 | TTTGGGACCTCCCCCATTTG (SEQ ID NO: 108) |
| 20 | ASO_20/20 | TTGGGACCTCCCCCATTTGA (SEQ ID NO: 109) |
| 20 | ASO_20/21 | TGGGACCTCCCCCATTTGAG (SEQ ID NO: 110) |
| 20 | ASO_20/22 | GGGACCTCCCCCATTTGAGA (SEQ ID NO: 111) |
| 20 | ASO 20/23 | GGACCTCCCCCATTTGAGAA (SEQ ID NO: 112) |
| 20 | ASO 20/24 | GACCTCCCCCATTTGAGAAG (SEQ ID NO: 113) |
| 20 | ASO_20/25 | ACCTCCCCCATTTGAGAAGG (SEQ ID NO: 114) |
| 20 | ASO 20/26 | CCTCCCCCATTTGAGAAGGG (SEQ ID NO: 115) |
| 20 | ASO_20/27 | CTCCCCCATTTGAGAAGGGC (SEQ ID NO: 116) |
| 20 | ASO 20/28 | TCCCCCATTTGAGAAGGGCA (SEQ ID NO: 117) |
| 20 | ASO 20/29 | CCCCCATTTGAGAAGGGCAT (SEQ ID NO: 118) |

Extended Data Table 1. Demographics of
COVID-19 Whole Blood RNA-Seq cohort

| | Control | | SARS-COV-2 | |
|---|---|---|---|---|
| n | 7 | | 8 | |
| Age, median (IQR) | 61 | (59, 66) | 64 | (58, 68) |
| Sex | | | | |
| Female, n (%) | 2 | (29) | 3 | (38) |
| Race | | | | |
| Non-White, n (%) | 4 | (57) | 7 | (88) |
| Hispanic, n (%) | 4 | (57) | 3 | (38) |
| Comorbidities | | | | |
| BMI, median (IQR) | 26.9 | (24.7, 28.6) | 27.6 | (24.8, 31.5) |
| Diabetes, n (%) | 1 | (14) | 5 | (71) |
| Hyperlipidemia, n (%) | 4 | (57) | 2 | (25) |
| Hypertension, n (%) | 3 | (43) | 5 | (63) |

Extended Data Table 2. LncRNAs differentially expressed
in both influenza A and SarsCoV2 infection

| | INFLUENZA A | | SARS-COV2 | |
|---|---|---|---|---|
| lincRNA | log2FoldChange | padj | log2FoldChange | padj |
| A2M-AS1 | −0.97 | 6.48E−03 | 7.54 | 2.93E−13 |
| AATBC | 1.37 | 1.04E−12 | 6.39 | 1.47E−12 |
| ABALON | 1.01 | 5.92E−03 | 8.56 | 2.78E−23 |
| ADAMTSL4-AS1 | 1.73 | 8.00E−12 | 7.43 | 2.68E−18 |
| ALMS1-IT1 | −0.84 | 4.33E−02 | 4.79 | 1.23E−03 |
| AOAH-IT1 | 1.68 | 3.28E−08 | 4.25 | 4.02E−04 |
| ARMCX5-GPRASP2 | −1.07 | 9.15E−03 | 5.83 | 8.72E−09 |
| ATP11A-AS1 | 0.89 | 9.90E−03 | 7.33 | 6.49E−15 |
| BASP1-AS1 | 1.29 | 6.46E−03 | 4.46 | 3.23E−06 |
| BISPR | 1.85 | 1.80E−22 | 6.31 | 2.60E−18 |
| C1QTNF7-AS1 | 0.96 | 4.49E−02 | 4.4 | 3.41E−05 |
| C5orf67 | 1.86 | 1.21E−06 | 4.5 | 3.87E−04 |
| CAHM | 0.9 | 3.00E−04 | 4.42 | 1.33E−05 |
| CARD8-AS1 | 0.84 | 1.04E−08 | 6.46 | 4.21E−52 |
| CCR5AS | 2.92 | 1.53E−23 | 4.8 | 5.77E−14 |
| CEROX1 | −0.85 | 8.56E−05 | 3.56 | 2.93E−02 |
| CFAP58-DT | 2.5 | 4.05E−19 | 7.26 | 3.01E−26 |
| CFLAR-AS1 | 1.14 | 1.54E−04 | 7.42 | 1.99E−28 |
| CHROMR | 1.88 | 1.58E−10 | 5.76 | 4.57E−20 |
| CKMT2-AS1 | −0.66 | 1.95E−02 | 5.48 | 2.05E−08 |
| CLRN1-AS1 | 1.27 | 4.21E−02 | 4.67 | 6.03E−04 |
| CYTOR | 1.35 | 2.16E−12 | 5.95 | 1.35E−40 |
| DHRS4-AS1 | −0.71 | 5.39E−07 | 7.56 | 1.48E−20 |
| DIAPH1-AS1 | 1.02 | 2.60E−02 | 7.2 | 4.65E−15 |
| DISC1-IT1 | 2.78 | 1.04E−09 | 5.92 | 1.52E−05 |
| DLEU2 | 1.1 | 1.20E−07 | 6.64 | 1.02E−87 |
| DOCK4-AS1 | 2.54 | 8.47E−07 | 5.04 | 1.64E−05 |
| DOCK8-AS1 | 1.31 | 2.94E−06 | 8.49 | 6.96E−55 |
| DPYD-AS1 | 1.11 | 4.21E−02 | 6.36 | 3.08E−10 |
| ECE1-AS1 | 0.59 | 1.92E−02 | 6.43 | 5.13E−12 |
| EIF1B-AS1 | 0.71 | 2.06E−02 | 7.16 | 9.94E−19 |
| EPB41L4A-AS1 | −0.64 | 8.31E−05 | 5.54 | 1.26E−21 |
| ERICH6-AS1 | −1.07 | 5.56E−04 | 5.04 | 2.70E−07 |
| FAM157C | 0.77 | 1.20E−03 | 7.55 | 1.81E−17 |
| FAM198B-AS1 | 0.94 | 5.11E−04 | 6.69 | 1.44E−13 |
| FOXN3-AS1 | 0.91 | 2.94E−04 | 4.43 | 6.49E−06 |
| FRY-AS1 | −0.89 | 1.10E−02 | 5.16 | 1.49E−07 |
| GABPB1-AS1 | −0.74 | 3.34E−04 | 7.96 | 8.95E−37 |

Extended Data Table 3. Genes differentially expressed
in THP-1 macrophages treated with GapCHROMR versus
GapCTRL after poly(I:C) stimulation for 8 h.

| Gene | log2FoldChange | padj |
|---|---|---|
| EGR1 | 3.31 | 8.14E−54 |
| STAT1 | −1.18 | 7.94E−41 |
| MS4A7 | −1.67 | 1.84E−39 |
| IL10RA | −2.06 | 5.02E−37 |
| MX2 | −1.44 | 3.40E−36 |
| JAG1 | 1.56 | 3.85E−35 |
| TOB1 | −1.65 | 4.27E−35 |
| IFI44L | −2.41 | 1.03E−33 |
| TIMP3 | 1.08 | 2.21E−33 |
| PARP9 | −1.39 | 9.64E−33 |
| TNFSF14 | 1.83 | 2.62E−32 |
| TRIB1 | 1.57 | 6.09E−32 |
| CXCL3 | 1.34 | 2.65E−31 |
| SLC2A5 | −1.3 | 5.62E−31 |
| OAS3 | −1 | 5.62E−31 |
| SLC20A1 | 1.17 | 1.05E−30 |
| PARP14 | −1.34 | 2.45E−30 |
| GPRIN3 | −1.92 | 1.37E−29 |
| DUSP10 | 1.1 | 1.66E−29 |
| SIGLEC1 | −1.14 | 4.60E−29 |
| EPSTI1 | −1.31 | 1.84E−28 |
| S1PR3 | 1.77 | 2.59E−28 |
| RGMB | 1.97 | 2.72E−28 |
| ECE1 | 1.5 | 3.34E−28 |
| XAF1 | −1.64 | 3.66E−28 |
| SAMD9L | −1.69 | 3.92E−28 |
| TRIM22 | −1.47 | 1.77E−27 |
| SPRED2 | 1.18 | 2.59E−27 |
| SLCO4A1 | 1.36 | 2.74E−27 |
| SAMHD1 | −1.35 | 4.30E−27 |
| MX1 | −1.45 | 1.23E−26 |
| GPR68 | 1.69 | 2.76E−26 |
| REL | −1.21 | 7.00E−26 |
| SAMD9 | −1.23 | 7.16E−25 |
| OAS2 | −1.06 | 7.16E−25 |
| TRIM14 | −1.18 | 2.47E−24 |
| ONECUT2 | −3.25 | 1.27E−23 |
| DNAJB5 | 1.31 | 2.24E−23 |
| POU2F2 | 1.23 | 4.44E−23 |
| FGFRL1 | 1.14 | 5.62E−23 |
| CITED4 | 1.76 | 5.79E−23 |
| NTSR1 | 1.5 | 9.63E−23 |
| STX1A | 1.63 | 1.61E−22 |
| MAF | −1.5 | 1.97E−22 |
| BMP2K | −1.07 | 2.00E−22 |
| RCBTB2 | −1.53 | 2.73E−22 |
| ITGA3 | 1.05 | 4.11E−22 |
| GDF15 | 2.04 | 4.42E−22 |
| DUSP5 | 1.49 | 1.02E−21 |
| HMGB3 | −1.24 | 1.61E−21 |
| MS4A14 | −1.67 | 2.30E−21 |
| GBP1 | −1.76 | 2.61E−21 |
| ZSWIM4 | 1.31 | 4.99E−21 |
| SIGLEC15 | 1.29 | 5.93E−21 |
| EIF2AK2 | −1.15 | 7.01E−21 |
| NRIP3 | 1.35 | 1.13E−20 |
| MERTK | −1.68 | 1.71E−20 |
| PIM1 | 1.61 | 2.42E−20 |
| CD36 | −1.41 | 2.66E−20 |
| SLC43A2 | −1.29 | 2.66E−20 |
| NPC1 | 1.21 | 3.22E−20 |
| ITGA5 | 1.01 | 3.26E−20 |
| LINC01094 | −2.42 | 5.31E−20 |
| THBD | 1.17 | 5.58E−20 |
| ITPRIP | 1.18 | 6.41E−20 |
| BHLHE41 | 1.29 | 7.46E−20 |
| AREG | 2.35 | 8.23E−20 |
| DDX60 | −1.26 | 8.35E−20 |
| COL6A3 | 1.28 | 9.40E−20 |
| MAFF | 1.48 | 9.49E−20 |
| SPHK1 | 1.42 | 9.54E−20 |
| TET2 | −1.05 | 1.40E−19 |
| S100A9 | −1.24 | 2.81E−19 |
| CORO2A | −1.74 | 3.06E−19 |
| EMP1 | 1.43 | 6.20E−19 |
| SIK1 | 1.37 | 7.15E−19 |
| DAPP1 | −1.81 | 8.82E−19 |
| GPR183 | −1.8 | 3.18E−18 |
| RUSC2 | 1.09 | 4.14E−18 |
| PLK3 | 1.37 | 4.31E−18 |
| MAP1S | 1.09 | 4.49E−18 |
| SMTN | 1.32 | 5.62E−18 |
| UCN2 | 1.21 | 6.92E−18 |
| IFIH1 | −1.19 | 8.61E−18 |
| IL36RN | 2.49 | 9.06E−18 |
| MAR1 | −1.77 | 9.83E−18 |
| IRF2BPL | 1.11 | 1.69E−17 |
| PLSCR1 | −1.08 | 4.20E−17 |
| KDM6B | 1.22 | 4.20E−17 |
| PTPRC | −1.36 | 1.14E−16 |
| GLUL | −2.69 | 1.16E−16 |
| MITF | −1.25 | 2.48E−16 |
| SLA | −1.17 | 4.15E−16 |
| FAM13A | −1.88 | 4.91E−16 |
| FGD4 | −2.31 | 5.99E−16 |
| PHLDA2 | 1.8 | 6.60E−16 |
| IRAK3 | −1.34 | 6.63E−16 |
| RASA3 | 1.01 | 7.85E−16 |
| IRS2 | 1.13 | 7.85E−16 |
| PEX5L | −1.52 | 8.85E−16 |
| KCNN4 | 1.18 | 1.22E−15 |
| RBL1 | −1.38 | 1.24E−15 |
| PLXDC2 | −1.45 | 1.34E−15 |
| C9orf66 | −1.32 | 1.54E−15 |
| FGR | 1.02 | 1.54E−15 |
| TREM2 | −1.29 | 1.76E−15 |
| ADORA2B | 1.79 | 1.76E−15 |
| BCL6 | 1.06 | 2.70E−15 |
| SIRPB2 | −3.19 | 3.41E−15 |
| BRCA2 | −1.37 | 5.52E−15 |
| CLEC7A | −3.08 | 6.64E−15 |
| TLR4 | −1.46 | 6.72E−15 |
| GUCY1A2 | −1.39 | 8.79E−15 |
| LIF | 2.46 | 8.88E−15 |
| LPAR6 | −1.41 | 9.41E−15 |
| S100A16 | 1.29 | 1.15E−14 |
| ZNF107 | −1.59 | 1.28E−14 |
| MOB3A | 1.08 | 1.31E−14 |
| S100A8 | −1.6 | 1.58E−14 |
| TNFSF10 | −2.15 | 3.94E−14 |
| APBB1IP | −1.54 | 4.11E−14 |
| SLC39A14 | 1.03 | 4.47E−14 |
| ICAM5 | 1.05 | 5.93E−14 |
| IFNGR1 | −1.01 | 6.26E−14 |
| PTPRO | −1.9 | 6.85E−14 |
| NRSN1 | 2.56 | 6.90E−14 |
| STK40 | 1.13 | 7.15E−14 |
| C10orf54 | −1.39 | 7.23E−14 |
| NRIP1 | −1.09 | 7.23E−14 |
| RHBDF1 | 1.43 | 7.42E−14 |
| STARD8 | 1.19 | 7.66E−14 |
| E2F7 | 1.04 | 8.62E−14 |
| STX3 | 1.09 | 9.65E−14 |
| MAP2K3 | 1.3 | 1.68E−13 |
| PDE4D | −1.76 | 1.79E−13 |
| HERC6 | −1.18 | 1.79E−13 |
| DACT1 | 1.85 | 2.14E−13 |
| FRY | −1 | 2.20E−13 |
| FCAR | 1.23 | 3.43E−13 |
| IFI44 | −1.25 | 4.69E−13 |
| CMPK2 | −1.17 | 5.31E−13 |
| FCGR3A | −1.74 | 5.96E−13 |
| LRRC8C | −1.26 | 7.31E−13 |
| GFPT2 | 1.38 | 7.32E−13 |
| UBASH3B | −1.26 | 8.06E−13 |
| OAF | 1.22 | 8.77E−13 |
| PTPRE | 1.04 | 1.16E−12 |
| LOC100294362 | −1.38 | 1.22E−12 |
| TMCC3 | 2.13 | 1.26E−12 |
| IER2 | 1.15 | 1.59E−12 |
| SERPINB8 | 1.03 | 1.67E−12 |
| ICOSLG | −1.21 | 1.73E−12 |
| PDGFRB | 1.54 | 1.74E−12 |
| CENPF | −1.19 | 1.84E−12 |
| JDP2 | −1.71 | 2.25E−12 |
| TLDC2 | −1.14 | 2.50E−12 |
| FYB | −1.96 | 3.05E−12 |
| TOMM34 | 1.03 | 3.16E−12 |
| FILIP1L | −1.17 | 4.70E−12 |
| LDLR | 1.71 | 5.17E−12 |
| MMS22L | −1.41 | 5.57E−12 |
| GBP4 | −1.44 | 6.67E−12 |
| SASH3 | −1.29 | 6.68E−12 |
| LINC00941 | 1.75 | 6.81E−12 |
| RHOU | −1.47 | 7.17E−12 |
| RARG | 1.09 | 7.17E−12 |
| CYP27A1 | −1.24 | 7.25E−12 |
| HS3ST3A1 | 1.26 | 7.25E−12 |
| GAB2 | −1.17 | 7.68E−12 |
| MGAT4A | −1.32 | 8.01E−12 |
| POLQ | −1.54 | 1.01E−11 |
| SNX2 | −1.07 | 1.02E−11 |
| DOK2 | −1.56 | 1.15E−11 |
| IPCEF1 | −1.33 | 1.35E−11 |
| HOPX | 1.09 | 1.67E−11 |
| CLSPN | −1.46 | 1.76E−11 |
| ALOX5 | −1.5 | 1.79E−11 |
| ME1 | −1.13 | 1.93E−11 |
| ISG15 | −1.29 | 2.98E−11 |
| TOP2A | −1.16 | 3.51E−11 |
| MNDA | −3.09 | 5.20E−11 |
| FZD5 | −2.02 | 7.27E−11 |
| TMEM200A | 1.24 | 7.50E−11 |
| GNG11 | 1.26 | 8.10E−11 |
| FAM105A | −2.56 | 9.34E−11 |
| MYEOV | 1.57 | 9.34E−11 |
| PCDH18 | −1.63 | 1.02E−10 |
| TNFRSF10D | 1.24 | 1.19E−10 |
| ATAD2 | −1.1 | 1.86E−10 |
| SH3RF1 | 1.03 | 2.10E−10 |
| ADAM28 | −1.15 | 2.31E−10 |
| ESCO2 | −1.42 | 2.38E−10 |
| DMWD | 1.02 | 2.65E−10 |
| SMC4 | −1.05 | 2.73E−10 |
| CCNE2 | −1.51 | 3.18E−10 |
| OASL | −1.1 | 3.50E−10 |
| DGKH | −1.1 | 4.51E−10 |
| SERPINE2 | 1.14 | 5.54E−10 |
| SMAGP | 2.08 | 5.84E−10 |
| CORO2B | 1.47 | 5.88E−10 |
| DLL1 | 1.58 | 5.97E−10 |
| SPRED3 | 1.87 | 7.35E−10 |
| DTL | −1.29 | 7.42E−10 |
| SNAI1 | −1.19 | 7.66E−10 |
| HELLS | −2.15 | 9.07E−10 |
| KL | −1.73 | 1.25E−09 |
| TMEM170B | −1.38 | 1.31E−09 |
| NLRC4 | −1.41 | 1.41E−09 |
| THSD7A | −1.47 | 1.47E−09 |
| ADRBK2 | −1.62 | 1.62E−09 |
| CCL3L1 | 3.45 | 1.63E−09 |
| STIL | −1.04 | 1.71E−09 |
| TNFSF13B | −1.91 | 1.86E−09 |
| EREG | 1.61 | 1.97E−09 |
| BRCA1 | −1.22 | 1.98E−09 |
| POLA1 | −1.37 | 2.08E−09 |
| TM4SF19-TCTEX1D2 | 1.76 | 2.60E−09 |
| GALM | −1.34 | 2.64E−09 |
| SLC8A1 | −1.24 | 3.01E−09 |
| CXorf21 | −1.54 | 3.03E−09 |
| TREML1 | −1.34 | 3.08E−09 |
| INTS7 | −1.02 | 3.16E−09 |
| ZNF93 | −1.66 | 3.19E−09 |
| CCL3L3 | 3.4 | 3.70E−09 |
| FCRLB | 1.18 | 3.91E−09 |
| ETS2 | −1.05 | 4.37E−09 |
| GBP5 | −1.06 | 4.50E−09 |
| MATK | 1.05 | 4.79E−09 |
| FAM111B | −1.81 | 6.21E−09 |
| METTL7B | 1.19 | 6.30E−09 |
| ARHGEF40 | −1.1 | 7.16E−09 |
| TBX21 | 3.5 | 8.01E−09 |
| TM4SF19 | 1.78 | 8.82E−09 |
| KRT79 | 1.41 | 1.02E−08 |
| MYRF | 1.08 | 1.03E−08 |
| STX17 | −1.08 | 1.13E−08 |
| MFSD2A | 1.67 | 1.29E−08 |
| GCNT1 | −2.03 | 1.34E−08 |
| MAST4 | 1.21 | 1.34E−08 |
| KNTC1 | −1.08 | 1.57E−08 |
| IL7R | −2.08 | 1.62E−08 |
| GAB3 | −1.41 | 1.73E−08 |
| MNT | 1.1 | 1.93E−08 |
| S100A12 | −2.66 | 2.01E−08 |
| ASPM | −1.12 | 2.03E−08 |
| FAM102A | 1.01 | 2.18E−08 |
| ARHGAP23 | 1.04 | 2.18E−08 |
| PMP22 | 1.85 | 2.23E−08 |
| NUSAP1 | −1.11 | 2.30E−08 |
| MLPH | 1.48 | 3.05E−08 |
| RAD54B | −1.55 | 3.32E−08 |
| TM4SF1 | 1.6 | 3.50E−08 |
| NCAPG | −1.16 | 3.74E−08 |
| EDNRB | −1.92 | 3.93E−08 |
| PKIB | −1.11 | 4.13E−08 |
| PRRX1 | 1.27 | 4.65E−08 |
| IL36B | 1.5 | 4.83E−08 |
| NCOA2 | −1.3 | 4.91E−08 |
| P2RY6 | −1.11 | 5.19E−08 |
| CD86 | −2.14 | 5.39E−08 |
| SNX30 | −1.08 | 5.79E−08 |
| CHIC2 | 1.44 | 6.13E−08 |
| NAV2 | 1.76 | 6.31E−08 |
| LRRK2 | −1.91 | 6.35E−08 |
| CITED2 | 1.75 | 6.52E−08 |
| SLC2A3 | 1.26 | 6.69E−08 |
| TBX15 | −1.61 | 6.83E−08 |
| ADCY7 | −1.13 | 6.94E−08 |
| UPP1 | 1.41 | 7.83E−08 |
| LOC100506585 | −2.04 | 8.67E−08 |
| JAK2 | −1.06 | 8.69E−08 |
| ZMIZ1-AS1 | 1.01 | 8.83E−08 |
| CEP152 | −1.47 | 9.08E−08 |
| SOX13 | 2.04 | 9.09E−08 |
| TM4SF19-AS1 | 1.85 | 9.45E−08 |
| OLR1 | −1.72 | 1.01E−07 |
| PLXNA2 | 1.77 | 1.08E−07 |
| BRCC3 | −1.14 | 1.20E−07 |
| CNTLN | −1.15 | 1.41E−07 |
| ATAD5 | −1.24 | 1.43E−07 |
| PAX8-AS1 | 1.29 | 1.53E−07 |
| GPR176 | 1.01 | 1.54E−07 |
| CDC42EP3 | 1.12 | 1.70E−07 |
| TRIB2 | 1.28 | 1.83E−07 |
| USP45 | −1.16 | 2.19E−07 |
| XKRX | 1.65 | 2.19E−07 |
| MLH3 | −1.07 | 2.30E−07 |
| RASSF2 | −2.27 | 2.69E−07 |
| IFIT3 | −1.95 | 2.69E−07 |
| CD84 | −1.39 | 2.71E−07 |
| FAM13A-AS1 | −1.9 | 3.09E−07 |
| RTTN | −1.34 | 3.20E−07 |
| DCBLD2 | 1.09 | 3.37E−07 |
| TCF7L2 | 1.18 | 4.23E−07 |
| EXO1 | −1.12 | 4.47E−07 |
| SDC1 | 1.36 | 4.61E−07 |
| ARPIN | −1.18 | 4.82E−07 |
| SRPX | 1.19 | 5.06E−07 |
| E2F8 | −1.4 | 5.28E−07 |
| IL1RN | 1.56 | 5.30E−07 |
| CYSLTR1 | −1.34 | 5.63E−07 |
| FAM109A | 1.07 | 5.68E−07 |
| USP18 | −1.08 | 6.27E−07 |
| TSC22D1 | 1.97 | 6.79E−07 |
| ZNF525 | −1.33 | 7.07E−07 |
| GPR65 | −1.03 | 7.68E−07 |
| RMI1 | −1.02 | 8.31E−07 |
| AVPI1 | −1.74 | 8.46E−07 |
| LAMA4 | 1.56 | 8.54E−07 |
| EMR3 | −1.73 | 8.66E−07 |
| MMP19 | 1.41 | 1.01E−06 |
| SH2B3 | 1.49 | 1.02E−06 |
| RGCC | 1.57 | 1.06E−06 |
| ZC3H12D | −1 | 1.08E−06 |
| SPECC1 | −1.39 | 1.11E−06 |
| WDR76 | −1.02 | 1.11E−06 |
| ZNF480 | −1.01 | 1.15E−06 |
| FAT4 | −1.17 | 1.16E−06 |
| MCM10 | −1.45 | 1.19E−06 |
| FRMD6 | 1.37 | 1.19E−06 |
| SLC25A37 | 1.06 | 1.20E−06 |
| FBLIM1 | 1.72 | 1.35E−06 |
| NOS2 | 2.79 | 1.40E−06 |
| RASAL1 | −2.64 | 1.54E−06 |
| PAPPA-AS1 | 1.4 | 1.72E−06 |
| PRL | −4.21 | 1.75E−06 |
| SESN3 | −1.55 | 1.75E−06 |
| GPR85 | −3.11 | 1.84E−06 |
| HERC5 | −1.31 | 1.84E−06 |
| MKI67 | −1.41 | 1.89E−06 |
| WWTR1 | 1.59 | 1.97E−06 |
| CDH2 | 1.34 | 2.01E−06 |
| PTPRF | −1.2 | 2.07E−06 |
| RBBP8 | −1.28 | 2.11E−06 |
| TFPI2 | 1.52 | 2.14E−06 |
| DYNC2H1 | −1.01 | 2.26E−06 |
| CALD1 | 1.49 | 2.31E−06 |
| CENPE | −1.27 | 2.40E−06 |
| COL22A1 | 1.98 | 2.55E−06 |
| TGFBI | −1.33 | 2.69E−06 |
| PPARG | −1.01 | 3.01E−06 |
| AIM1 | −1.01 | 3.07E−06 |
| GPRC5C | 1.29 | 3.15E−06 |
| BUB1 | −1.08 | 3.49E−06 |
| ZNF366 | −1.56 | 3.58E−06 |
| ZGRF1 | −1.22 | 3.62E−06 |
| ZNF780B | −1.22 | 4.17E−06 |
| TYMS | −1.1 | 4.26E−06 |
| SHCBP1 | −1.05 | 4.28E−06 |
| CTH | 1.77 | 4.28E−06 |
| DOT1L | 1.29 | 4.35E−06 |
| ANKRD45 | −2.19 | 4.72E−06 |
| SNCB | 1.72 | 5.05E−06 |
| KIF23 | −1.16 | 5.41E−06 |
| TEC | −1.23 | 5.59E−06 |
| LOC100288637 | −1.39 | 5.70E−06 |
| STOX2 | −1.39 | 5.89E−06 |
| EHD1 | 1.37 | 5.91E−06 |
| PLAUR | 1.87 | 6.10E−06 |
| RASSF4 | −1.13 | 6.32E−06 |
| FZD4 | 1.56 | 6.64E−06 |
| LOXL2 | 1.06 | 7.77E−06 |
| IFIT1 | −1.73 | 7.82E−06 |
| BLM | −1.23 | 7.83E−06 |
| GCH1 | 1.4 | 7.83E−06 |
| PTGS2 | 1.79 | 7.86E−06 |
| RASGRP3 | −1.05 | 8.09E−06 |
| KIAA0101 | −1.3 | 8.56E−06 |
| AMY2B | −1.7 | 9.12E−06 |
| CDCA2 | −1.15 | 9.12E−06 |
| ETS1 | 1.27 | 9.73E−06 |
| DDX58 | −1.56 | 1.02E−05 |
| USP53 | 1.05 | 1.06E−05 |
| FMN1 | −1.06 | 1.14E−05 |
| CACNA2D3 | 1.44 | 1.17E−05 |
| HIST1H4I | −1.07 | 1.19E−05 |
| NR1D1 | 1.42 | 1.20E−05 |
| SCIMP | −3.06 | 1.22E−05 |
| ABI3 | −2.3 | 1.25E−05 |
| RGS16 | 2.2 | 1.28E−05 |
| CENPU | −1.12 | 1.29E−05 |
| ATOH8 | −2.32 | 1.34E−05 |
| LAT | 1.31 | 1.34E−05 |
| LYRM7 | −1.16 | 1.35E−05 |
| CSF3 | 1.72 | 1.39E−05 |
| ANTXR1 | −1.11 | 1.41E−05 |
| SPRY4 | 2.79 | 1.46E−05 |
| CEP78 | −1.21 | 1.50E−05 |
| DIRAS2 | 1.95 | 1.59E−05 |
| RAB39A | −1.16 | 1.62E−05 |
| PTRF | 1.06 | 1.72E−05 |
| VIPR2 | 2.37 | 1.72E−05 |
| KIF11 | −1.21 | 1.80E−05 |
| PPP1R15A | 1.92 | 2.12E−05 |
| FKBP10 | 1.36 | 2.15E−05 |
| LOC729083 | −1.81 | 2.17E−05 |
| MDGA1 | 1.73 | 2.18E−05 |
| GRIN3A | −2.04 | 2.23E−05 |
| HIST2H3D | −1.15 | 2.26E−05 |
| BUB1B | −1.13 | 2.26E−05 |
| TLR8 | −2.45 | 2.45E−05 |
| CEMIP | 1.01 | 2.45E−05 |
| RPLP0P2 | 1.08 | 2.45E−05 |
| ENOX1 | −1.06 | 2.67E−05 |
| TIGD2 | −1.82 | 2.69E−05 |
| RAP1GAP | 1.01 | 2.74E−05 |
| HBEGF | 1.75 | 2.92E−05 |
| MYBL1 | −1.88 | 3.00E−05 |
| SSC4D | −1.08 | 3.11E−05 |
| LOC153684 | −1.45 | 3.24E−05 |
| SDS | −2.3 | 3.34E−05 |
| PDE4B | −1.22 | 3.35E−05 |
| CCDC144NL | −1.03 | 3.37E−05 |
| IRGQ | 1.18 | 3.38E−05 |
| KIF21B | 2.09 | 3.38E−05 |
| ABCB10 | −1.13 | 3.39E−05 |
| LINC00460 | 2.34 | 3.49E−05 |
| IFIT2 | −2.13 | 3.62E−05 |
| E2F2 | −1.62 | 3.67E−05 |
| SPDL1 | −1.01 | 3.72E−05 |
| PTP4A3 | 1.23 | 3.80E−05 |
| PRSS35 | −2.65 | 3.96E−05 |
| TMEM255A | −1.11 | 4.01E−05 |
| SEMA3G | −1.98 | 4.05E−05 |
| SERPINE1 | 1.48 | 4.38E−05 |
| NFATC2 | 1.11 | 4.51E−05 |
| MMP3 | 1.93 | 4.51E−05 |
| GEM | 1.86 | 4.59E−05 |
| PLK4 | −1.08 | 4.79E−05 |
| FOSL1 | 1.57 | 5.23E−05 |
| ZNF100 | −1.25 | 5.34E−05 |
| FOSB | 2.83 | 5.35E−05 |
| APCDD1L | 1.46 | 6.15E−05 |
| HTR7 | 1.09 | 6.20E−05 |
| SAMSN1 | −1.95 | 6.37E−05 |
| ULK2 | −1.13 | 6.38E−05 |
| PLD6 | 1.19 | 6.46E−05 |
| RAB27A | −1.03 | 6.61E−05 |
| CCL3 | 2.16 | 6.77E−05 |
| CDR1 | 1.06 | 7.14E−05 |
| GPR3 | 1.65 | 7.55E−05 |
| ADORA2A | 1.16 | 8.12E−05 |
| CDC7 | −1.24 | 8.15E−05 |
| HPCAL1 | 1.16 | 8.17E−05 |
| TSIX | 1.84 | 8.45E−05 |
| DUSP7 | 1.18 | 8.62E−05 |
| IRAK2 | 1.58 | 8.96E−05 |
| LINC01410 | −1.5 | 9.13E−05 |
| IL1B | 2.93 | 9.15E−05 |
| DCLRE1C | −1.13 | 9.32E−05 |
| MPEG1 | −1.9 | 9.81E−05 |
| GPR56 | 1.48 | 1.02E−04 |
| SPRY2 | 2.19 | 1.05E−04 |
| HGD | −1.43 | 1.09E−04 |
| TYMSOS | −1.46 | 1.10E−04 |
| ZNF431 | −1.19 | 1.14E−04 |
| TNFSF15 | 2.16 | 1.16E−04 |
| CEP128 | −1.41 | 1.27E−04 |
| CSF1 | 1.83 | 1.27E−04 |
| GDAP1 | −1.56 | 1.40E−04 |
| POU4F2 | 1.05 | 1.46E−04 |
| RSAD2 | −1.9 | 1.54E−04 |
| RNASE2 | −1.03 | 1.61E−04 |
| CXCL8 | 1.51 | 1.66E−04 |
| KLHL29 | −1.01 | 1.68E−04 |
| KANK1 | 1.27 | 1.69E−04 |
| SGOL1 | −1.06 | 1.79E−04 |
| CHAF1B | −1.1 | 1.84E−04 |
| TBX3 | 1.21 | 1.85E−04 |
| FES | −1.17 | 2.04E−04 |
| DYNLL1-AS1 | 1.07 | 2.04E−04 |
| TCHH | −1.59 | 2.06E−04 |
| RGS5 | −1.51 | 2.09E−04 |
| PRIM1 | −1 | 2.19E−04 |
| KIF15 | −1.21 | 2.29E−04 |
| PTAFR | −1.19 | 2.31E−04 |
| GPX3 | 1.19 | 2.31E−04 |
| OSMR | 1.04 | 2.38E−04 |
| CYBB | −1.92 | 2.50E−04 |
| FPR1 | −4.73 | 2.54E−04 |
| FGD2 | −1.81 | 2.61E−04 |
| NT5E | 1.01 | 2.65E−04 |
| GJA1 | 1.13 | 2.65E−04 |
| PKDCC | −1.92 | 2.66E−04 |
| ZNF678 | −1.01 | 2.66E−04 |
| BAALCOS | 1.62 | 2.77E−04 |
| FLNC | 1.09 | 2.87E−04 |
| MEG3 | 1.62 | 2.92E−04 |
| RTP4 | −1 | 2.98E−04 |
| DDIT4L | 2 | 3.10E−04 |
| SORL1 | −2.09 | 3.14E−04 |
| MAS1 | 2.27 | 3.15E−04 |
| ZNF33A | −1.39 | 3.16E−04 |
| UBALD2 | 1.35 | 3.27E−04 |
| TRHDE-AS1 | −1.03 | 3.43E−04 |
| PRSS22 | 2.49 | 3.52E−04 |
| IL33 | 1.23 | 3.53E−04 |
| KDELC2 | −1.01 | 3.66E−04 |
| PCDH1 | 1.4 | 3.68E−04 |
| C5orf42 | −1.18 | 3.84E−04 |
| TSPAN13 | 1.22 | 3.84E−04 |
| SNX9 | 1.56 | 3.88E−04 |
| CSRNP1 | 1.14 | 3.96E−04 |
| NR2F1 | 1.45 | 4.07E−04 |
| ECM2 | −2.39 | 4.39E−04 |
| KIAA1524 | −1.01 | 4.44E−04 |
| POSTN | 1.41 | 4.54E−04 |
| SKA1 | −1.13 | 4.61E−04 |
| HIST1H4A | −1.85 | 4.64E−04 |
| CCDC80 | 1.35 | 4.68E−04 |
| FCGR2C | −1.98 | 4.71E−04 |
| C5 | −1.19 | 4.74E−04 |
| LILRB2 | −2.64 | 4.83E−04 |
| WTAPP1 | 1.48 | 4.83E−04 |
| TMEM86A | −1.1 | 4.86E−04 |
| SEMA7A | 1.47 | 5.05E−04 |
| LOC100507065 | 1.54 | 5.14E−04 |
| NEDD4L | 1.56 | 5.16E−04 |
| XIRP1 | 1.89 | 5.34E−04 |
| NOD1 | −1.04 | 5.35E−04 |
| ZNF43 | −1.42 | 5.46E−04 |
| LINC01050 | 1.12 | 5.78E−04 |
| RASL12 | 2.58 | 5.96E−04 |
| PPARGC1A | −2.4 | 6.00E−04 |
| COLEC12 | −2.02 | 6.18E−04 |
| ZNF273 | −1.12 | 6.30E−04 |
| HIST1H2BI | −1.93 | 6.41E−04 |
| ODF3B | −1.41 | 6.49E−04 |
| PHLDA1 | 1.25 | 6.77E−04 |
| KLRK1 | 3.82 | 7.09E−04 |
| LBH | 1.1 | 7.12E−04 |
| F2RL1 | 1.17 | 7.24E−04 |
| HIST1H2AM | −1.02 | 7.27E−04 |
| SGMS1 | 1.04 | 7.28E−04 |
| LOC728084 | 1.33 | 7.48E−04 |
| ZNF467 | −1.9 | 7.49E−04 |
| CEBPA | −1.11 | 7.57E−04 |
| RGMB-AS1 | 1.69 | 7.79E−04 |
| ELTD1 | 1.74 | 7.87E−04 |
| RBM41 | −1.1 | 7.88E−04 |
| DRAXIN | 2.61 | 8.02E−04 |
| NEURL1B | −1.66 | 8.27E−04 |
| CXCL10 | −2.67 | 8.75E−04 |
| DUSP6 | 1.15 | 8.81E−04 |
| B3GNT5 | −1.42 | 9.16E−04 |
| ITGB3 | 1.46 | 9.34E−04 |
| KLRC4-KLRK1 | 3.6 | 9.68E−04 |
| DKK3 | 1.44 | 9.80E−04 |
| ZNF138 | −1.39 | 9.84E−04 |
| ZNF675 | −1.02 | 1.02E−03 |
| ARNT2 | 1.01 | 1.04E−03 |
| KCNQ3 | −1.27 | 1.11E−03 |
| SECTM1 | −1.37 | 1.11E−03 |
| SLC16A6 | 1.22 | 1.12E−03 |
| KIAA0513 | −1.27 | 1.12E−03 |
| POLE2 | −1.12 | 1.14E−03 |
| SLC15A2 | −1.19 | 1.15E−03 |
| IRX3 | 1 | 1.19E−03 |
| RAD51AP1 | −1.73 | 1.21E−03 |
| COL24A1 | −1.18 | 1.21E−03 |
| STAT4 | −1.95 | 1.33E−03 |
| KRT78 | 1.41 | 1.36E−03 |
| COL5A2 | 1.09 | 1.37E−03 |
| FOXP4 | −1.35 | 1.37E−03 |
| APBB2 | 1.09 | 1.39E−03 |
| FMO5 | −1.68 | 1.40E−03 |
| ZNF441 | −1.41 | 1.42E−03 |
| SEC31B | −1.51 | 1.42E−03 |
| SRC | 1.34 | 1.44E−03 |
| UPF3B | −1 | 1.45E−03 |
| UCHL1 | 1.41 | 1.47E−03 |
| HMGA2 | 2.01 | 1.48E−03 |
| PDGFA | 1.52 | 1.48E−03 |
| HS3ST3B1 | 1.38 | 1.49E−03 |
| CD101 | −2.55 | 1.49E−03 |
| FZD8 | 1.24 | 1.54E−03 |
| AKAP12 | 1.09 | 1.57E−03 |
| EFNB2 | 2.06 | 1.58E−03 |
| KLF15 | −2.27 | 1.59E−03 |
| CRISPLD2 | −1.11 | 1.64E−03 |
| DUSP4 | 1.26 | 1.69E−03 |
| DLC1 | 1.44 | 1.72E−03 |
| SERPINA9 | 1.57 | 1.80E−03 |
| AEBP1 | 1.21 | 1.88E−03 |
| HIST4H4 | −2.18 | 1.89E−03 |
| MMP1 | 1.44 | 1.92E−03 |
| XPA | −1.19 | 1.98E−03 |
| GPCPD1 | −1 | 2.04E−03 |
| STC1 | 2.52 | 2.07E−03 |
| C18orf54 | −1.42 | 2.15E−03 |
| RCSD1 | −1.01 | 2.25E−03 |
| SQLE | 1.34 | 2.31E−03 |
| KIAA2022 | −2.4 | 2.36E−03 |
| ZNF577 | −1.11 | 2.40E−03 |
| IL1A | 1.15 | 2.42E−03 |
| SENCR | −1.3 | 2.43E−03 |
| STARD4 | 1.1 | 2.59E−03 |
| DSEL | 1.51 | 2.60E−03 |
| CCND1 | 1.18 | 2.62E−03 |
| ROBO4 | 1.11 | 2.74E−03 |
| LOC284454 | 1.04 | 2.78E−03 |
| LINC01204 | 2.34 | 2.80E−03 |
| ZNF85 | −1.2 | 2.84E−03 |
| LAMB2 | 1.07 | 2.95E−03 |
| ADAMTS12 | 1.31 | 2.96E−03 |
| CBR3 | −1.32 | 3.03E−03 |
| KCNF1 | 2.23 | 3.08E−03 |
| TUBA3FP | −1.6 | 3.09E−03 |
| RGS4 | 1.13 | 3.19E−03 |
| SPTA1 | −1.62 | 3.22E−03 |
| NUMB | 1.03 | 3.24E−03 |
| CENPI | −1.59 | 3.25E−03 |
| VCAN | −1.1 | 3.30E−03 |
| RNF125 | −1.51 | 3.41E−03 |
| LOC100272217 | 1.16 | 3.44E−03 |
| LIPG | 1.39 | 3.70E−03 |
| TP73 | −1.16 | 3.70E−03 |
| CYP7B1 | −1.12 | 3.75E−03 |
| LARP6 | 1.41 | 3.89E−03 |
| MYCT1 | 1.5 | 3.92E−03 |
| CKMT2 | −2.27 | 3.93E−03 |
| ALOX12P2 | −1.9 | 4.13E−03 |
| KIAA1549L | 1.7 | 4.22E−03 |
| PCDHB4 | −3.39 | 4.29E−03 |
| CCL8 | −2.76 | 4.40E−03 |
| FAM83G | 1.09 | 4.41E−03 |
| FA2H | 1.61 | 4.42E−03 |
| SAMD3 | 1.45 | 4.45E−03 |
| LRRC17 | 1.3 | 4.49E−03 |
| FPR2 | −2.5 | 4.61E−03 |
| PTGIS | 1.4 | 4.63E−03 |
| ADORA3 | −1.52 | 4.64E−03 |
| C12orf75 | 1.24 | 4.65E−03 |
| QRICH2 | 1.25 | 4.67E−03 |
| ASPN | −2.26 | 4.78E−03 |
| NGF | 2.9 | 4.81E−03 |
| OLFML2B | −1.18 | 4.88E−03 |
| PDGFRA | 1.55 | 5.11E−03 |
| F2R | 1.06 | 5.14E−03 |
| FSBP | −1.71 | 5.17E−03 |
| IL24 | 1.49 | 5.18E−03 |
| LOC102724323 | −2.47 | 5.25E−03 |
| DLGAP1-AS2 | 1.55 | 5.25E−03 |
| RPSAP52 | 1.99 | 5.28E−03 |
| C9orf47 | 1.05 | 5.30E−03 |
| FRMPD2 | 1.95 | 5.31E−03 |
| OXER1 | −1.79 | 5.32E−03 |
| CCDC15 | −1.18 | 5.37E−03 |
| C1orf162 | −1.18 | 5.49E−03 |
| LOXL4 | −1.29 | 5.70E−03 |
| TLR3 | −2.1 | 5.93E−03 |
| CD48 | −1.27 | 5.94E−03 |
| KCNJ11 | 2.24 | 6.14E−03 |
| BGN | 1.17 | 6.29E−03 |
| IL21R | −1.1 | 6.48E−03 |
| TRIM9 | 1.01 | 6.58E−03 |
| CXCL11 | −2.35 | 6.60E−03 |
| TNFRSF9 | −1.47 | 6.66E−03 |
| NRG1 | 1.75 | 6.68E−03 |
| RIBC2 | −1.56 | 6.77E−03 |
| MAGI3 | −1.31 | 6.85E−03 |
| KIF25-AS1 | −1.13 | 6.86E−03 |
| DPY19L2P2 | −2.62 | 6.95E−03 |
| SIM2 | 1.47 | 7.02E−03 |
| CCND2 | 1.5 | 7.13E−03 |
| CASK | 1.61 | 7.15E−03 |
| ADAMTS1 | 1.07 | 7.15E−03 |
| CHST13 | −1.3 | 7.23E−03 |
| NAV2-AS2 | 3.3 | 7.31E−03 |
| GRIA3 | −1.91 | 7.42E−03 |
| KBTBD7 | −1.03 | 7.42E−03 |
| C7orf31 | −1.23 | 7.45E−03 |
| OCSTAMP | 3.01 | 7.59E−03 |
| SLC16A10 | −1.23 | 7.62E−03 |
| HES4 | 1.47 | 7.63E−03 |
| SLC35E4 | 1.08 | 7.67E−03 |
| ZNF37BP | −1.66 | 7.71E−03 |
| LANCL3 | −1.44 | 7.72E−03 |
| CFH | 1.26 | 7.81E−03 |
| UPK3B | −2.91 | 7.98E−03 |
| ETV7 | −1.1 | 8.05E−03 |
| LOC101929586 | 2.14 | 8.06E−03 |
| IL10RB-AS1 | −1.21 | 8.12E−03 |
| GABBR2 | 1.49 | 8.21E−03 |
| CYP26B1 | 2.64 | 8.31E−03 |
| TMEM14E | −1.12 | 8.33E−03 |
| GBP2 | −1.05 | 8.33E−03 |
| MTCL1 | 1.05 | 8.46E−03 |
| BCL11B | 1.09 | 8.76E−03 |
| DBIL5P2 | −3.5 | 8.79E−03 |
| FCGR1C | −1.64 | 8.81E−03 |
| RRS1-AS1 | −2.29 | 8.86E−03 |
| PMFBP1 | −1.19 | 8.90E−03 |
| C5AR2 | 1.04 | 9.08E−03 |
| BRIP1 | −1.15 | 9.20E−03 |
| MIR612 | −2.11 | 9.29E−03 |
| NIM1K | −1.14 | 9.35E−03 |
| SIGLEC11 | −1.5 | 9.36E−03 |
| MFAP5 | 1.35 | 9.42E−03 |
| MTSS1 | 1.22 | 9.67E−03 |
| PAMR1 | 1.8 | 9.72E−03 |
| C1QTNF1 | 1.45 | 9.79E−03 |
| ACKR3 | 1.39 | 1.00E−02 |
| CLDN14 | 1.87 | 1.01E−02 |
| LINC00968 | −2.47 | 1.01E−02 |
| BCAS4 | 2.44 | 1.01E−02 |
| EVA1A | 1.49 | 1.02E−02 |
| DLGAP5 | −1.35 | 1.03E−02 |
| PTPN22 | −1.47 | 1.03E−02 |
| VAMP5 | −1.34 | 1.04E−02 |
| ZNF503 | −1.13 | 1.04E−02 |
| RASD2 | −2.28 | 1.06E−02 |
| TTC26 | −1.03 | 1.08E−02 |
| PLK2 | 1.03 | 1.08E−02 |
| PRICKLE1 | −1.09 | 1.09E−02 |
| COL1A2 | 1.4 | 1.10E−02 |
| THEM6 | −1.28 | 1.13E−02 |
| PLCD3 | 1.08 | 1.14E−02 |
| TET1 | −1.97 | 1.15E−02 |
| GPR18 | −1.05 | 1.16E−02 |
| PROX2 | −1.45 | 1.16E−02 |
| ZNF700 | −1.05 | 1.16E−02 |
| FAM20A | −1.52 | 1.18E−02 |
| PPP1R14C | 1.14 | 1.19E−02 |
| C19orf57 | −1.57 | 1.19E−02 |
| CDH11 | 1.45 | 1.20E−02 |
| SYN1 | 1.32 | 1.20E−02 |
| FAM161A | −1.97 | 1.20E−02 |
| FAM65B | 1.49 | 1.21E−02 |
| EGR3 | 2.41 | 1.21E−02 |
| GAPT | −2.21 | 1.22E−02 |
| LINC00278 | −1.35 | 1.27E−02 |
| INHBA | 1.38 | 1.27E−02 |
| RGAG4 | −1.03 | 1.28E−02 |
| LOC101927543 | −1.09 | 1.28E−02 |
| DKFZP434K028 | 3.22 | 1.31E−02 |
| CHN2 | −1.74 | 1.31E−02 |
| LOC101928790 | −2.41 | 1.32E−02 |
| ZFP14 | −1.29 | 1.32E−02 |
| USP32P2 | −1.11 | 1.32E−02 |
| COL5A1 | 1.34 | 1.32E−02 |
| LOXHD1 | 1.02 | 1.32E−02 |
| CD180 | −1.49 | 1.34E−02 |
| FBN1 | 1.39 | 1.37E−02 |
| LOC642757 | −2.31 | 1.37E−02 |
| ZNF546 | −1.02 | 1.38E−02 |
| MROH9 | 1.66 | 1.39E−02 |
| LDHAL6B | 1.12 | 1.41E−02 |
| MMP10 | 1.18 | 1.43E−02 |
| CNN3 | 1.1 | 1.46E−02 |
| LOC100288798 | −2.41 | 1.47E−02 |
| AMIGO2 | 1.28 | 1.47E−02 |
| HESX1 | −2.86 | 1.52E−02 |
| LOC729683 | 1.09 | 1.54E−02 |
| GBP1P1 | −2.31 | 1.54E−02 |
| LOC202181 | −1.14 | 1.58E−02 |
| HSPA4L | −1.09 | 1.61E−02 |
| ZNF630 | −3.42 | 1.65E−02 |
| CDCA7 | −1.05 | 1.67E−02 |
| PAPPA | 1.36 | 1.68E−02 |
| CDH13 | 1.17 | 1.72E−02 |
| HMMR-AS1 | −3.09 | 1.73E−02 |
| DNAH17-AS1 | −1.2 | 1.76E−02 |
| CTGF | 1.4 | 1.80E−02 |
| LOC641367 | −1.83 | 1.81E−02 |
| PTGER3 | 1.46 | 1.81E−02 |
| MGC39584 | −2.72 | 1.84E−02 |
| PSMD6-AS2 | −1.01 | 1.86E−02 |
| FSTL1 | 1.13 | 1.87E−02 |
| NKD1 | −1.05 | 1.93E−02 |
| KCNIP3 | −2.08 | 1.94E−02 |
| EGR2 | 2.09 | 1.99E−02 |
| PLAC8 | −1.18 | 1.99E−02 |
| GCNT2 | −1.33 | 2.08E−02 |
| IL21R-AS1 | −1.45 | 2.09E−02 |
| MIR6835 | 2.1 | 2.09E−02 |
| TRNP1 | 2.04 | 2.09E−02 |
| SLC24A4 | −1.38 | 2.10E−02 |
| CCR7 | 1.31 | 2.11E−02 |
| WNT7B | 1.73 | 2.12E−02 |
| MAP2 | −1.32 | 2.17E−02 |
| OCLM | −2.29 | 2.18E−02 |
| SH3RF3 | 1.11 | 2.18E−02 |
| SLC24A3 | 1.79 | 2.18E−02 |
| THBS1 | 1.07 | 2.20E−02 |
| ZNF44 | −1.25 | 2.22E−02 |
| MAP1LC3C | −2.46 | 2.25E−02 |
| MYBL2 | −1.03 | 2.27E−02 |
| SPARC | 1.3 | 2.31E−02 |
| ZNF680 | −1.04 | 2.37E−02 |
| CENPM | −1.06 | 2.38E−02 |
| RBM26-AS1 | 1.07 | 2.40E−02 |
| LINC00964 | 1.5 | 2.43E−02 |
| LOC148696 | −1.03 | 2.45E−02 |
| FGL2 | −1.08 | 2.45E−02 |
| CALB1 | −2.65 | 2.46E−02 |
| GVINP1 | −2.08 | 2.47E−02 |
| SHISA7 | −2.22 | 2.49E−02 |
| COL4A1 | 1.25 | 2.52E−02 |
| SEZ6 | −1.06 | 2.53E−02 |
| MINOS1P1 | −1.38 | 2.57E−02 |
| ZNF519 | −1.07 | 2.61E−02 |
| COL3A1 | 1.29 | 2.62E−02 |
| RAB20 | −1.85 | 2.63E−02 |
| ZNF331 | −1.17 | 2.70E−02 |
| COL1A1 | 1.18 | 2.71E−02 |
| XIST | 1.45 | 2.72E−02 |
| CIITA | −2.63 | 2.73E−02 |
| NSUN6 | −1.19 | 2.73E−02 |
| NKX3-1 | 1.9 | 2.73E−02 |
| PCDHAC2 | −1.1 | 2.77E−02 |
| OLFML3 | −1.37 | 2.79E−02 |
| ZNF99 | −3.13 | 2.79E−02 |
| GPR141 | −1.96 | 2.82E−02 |
| XK | −1.86 | 2.87E−02 |
| PLD4 | −1.14 | 2.97E−02 |
| GALNT9 | 1.23 | 2.97E−02 |
| P2RX6 | −1.81 | 2.98E−02 |
| CSMD3 | −2.82 | 3.01E−02 |
| SERPINB2 | 1.91 | 3.05E−02 |
| PTHLH | 1.38 | 3.09E−02 |
| FARP1 | 1.45 | 3.17E−02 |
| KLF5 | 1.54 | 3.20E−02 |
| LOC101927100 | −1.19 | 3.20E−02 |
| MEDAG | 1.43 | 3.21E−02 |
| FLRT1 | −1.38 | 3.26E−02 |
| LINC00342 | −1.41 | 3.28E−02 |
| DAPK2 | −1.04 | 3.30E−02 |
| CLDN11 | 1.11 | 3.31E−02 |
| HAS2 | 1.17 | 3.33E−02 |
| APLN | −1.61 | 3.36E−02 |
| FTX | −1.29 | 3.41E−02 |
| LOC100505736 | −1.41 | 3.49E−02 |
| ARHGAP29 | 1.05 | 3.51E−02 |
| NHSL2 | −1.67 | 3.55E−02 |
| ABCG2 | −1.12 | 3.55E−02 |
| SCN5A | 2.15 | 3.56E−02 |
| PCOLCE | 1.1 | 3.56E−02 |
| NFIB | 1.03 | 3.59E−02 |
| TUSC3 | 1.49 | 3.68E−02 |
| C16orf74 | 1.77 | 3.70E−02 |
| LOC728637 | −1.66 | 3.76E−02 |
| BANK1 | −2.06 | 3.79E−02 |
| MIR7844 | 1.68 | 3.83E−02 |
| TEDDM1 | −1.79 | 3.88E−02 |
| LOC158960 | −1.79 | 3.90E−02 |
| ERCC6L | −1.06 | 3.92E−02 |
| C9orf139 | −1.56 | 3.92E−02 |
| SRSF12 | −2.06 | 3.97E−02 |
| NTN4 | 1.39 | 3.98E−02 |
| ITGBL1 | 2.32 | 4.01E−02 |
| CCDC171 | −1.64 | 4.02E−02 |
| COL4A2-AS1 | 1.5 | 4.02E−02 |
| MCOLN3 | −1.76 | 4.02E−02 |
| ANKRD23 | −1.11 | 4.03E−02 |
| TGFBR3 | 1.04 | 4.05E−02 |
| C22orf34 | −1.19 | 4.10E−02 |
| LOC101929723 | −2.12 | 4.11E−02 |
| ARHGAP31-AS1 | −2.51 | 4.13E−02 |
| ARSJ | 1.45 | 4.13E−02 |
| TMOD2 | −1.19 | 4.16E−02 |
| PRICKLE2 | 1.63 | 4.16E−02 |
| HSPA6 | −1.75 | 4.24E−02 |
| TGFB3 | 1.54 | 4.27E−02 |
| LOC101928766 | 2.08 | 4.27E−02 |
| NR4A1 | −1.59 | 4.30E−02 |
| KLLN | −1.48 | 4.30E−02 |
| RAB3B | 1.14 | 4.32E−02 |
| EPB41L4B | 1.52 | 4.34E−02 |
| ZNF485 | −1.52 | 4.35E−02 |
| FRMD5 | −1.13 | 4.37E−02 |
| CCL20 | 1.25 | 4.41E−02 |
| LINC00702 | 1.71 | 4.44E−02 |
| FPR3 | −1.88 | 4.45E−02 |
| PDE10A | 2.38 | 4.45E−02 |
| NOXA1 | −2.32 | 4.51E−02 |
| MIR7848 | −2.51 | 4.52E−02 |
| KIAA1841 | −1.24 | 4.65E−02 |
| ALX4 | −2.06 | 4.85E−02 |
| SLC46A2 | −1.81 | 4.88E−02 |
| EDIL3 | 1.09 | 4.89E−02 |
| MIR4725 | 1.58 | 4.92E−02 |
| SERPINE3 | −1.02 | 4.95E−02 |

Extended Data Table 4. Genes differentially expressed
in THP-1macrophages treated with GapCHROMR versus
GapCTRL after LPS stimulation for 3 h.

| Gene | log2FoldChange | padj |
|---|---|---|
| SNX3 | −2.32 | 3.16E-145 |
| CXCL10 | −2.24 | 4.14E-134 |
| CXCL11 | −2.18 | 4.69E-112 |
| SLC11A2 | −1.93 | 6.14E-109 |
| APP | −1.78 | 6.41E-98 |
| SECISBP2 | −2.57 | 5.73E-85 |
| CCL2 | −1.94 | 1.55E-67 |
| IDO1 | −1.7 | 1.75E-67 |
| CXCL9 | −2.87 | 7.73E-67 |
| IL6 | −2.49 | 4.82E-66 |
| FGFR1OP | −2.59 | 3.61E-64 |
| CCL8 | −1.94 | 1.27E-60 |
| NDUFV1 | −1.59 | 7.37E-58 |
| LOC101927 | −2.91 | 5.24E-56 |
| GPX1 | −1.37 | 7.79E-55 |
| ME3 | −2.47 | 1.52E-48 |
| SMARCC1 | −1.41 | 8.93E-45 |
| IFIT2 | −1.48 | 1.29E-44 |
| COMMD10 | −2.36 | 1.97E-44 |
| CCDC30 | 1.6 | 1.21E-42 |
| TNFSF10 | −1.54 | 4.60E-42 |
| CBWD1 | −1.63 | 2.33E-40 |
| RSAD2 | −1.34 | 1.08E-39 |
| RPL34 | −1.16 | 2.29E-39 |
| THBD | 1.3 | 3.04E-39 |
| CBWD2 | −1.89 | 3.66E-37 |
| TTC7B | −1.57 | 3.48E-36 |
| GBP5 | −1.07 | 8.82E-36 |
| BNIP3L | −1.76 | 2.72E-35 |
| IRGQ | 1.05 | 2.98E-35 |
| SAMD4A | −1.1 | 4.91E-35 |
| IFIT1 | −1.07 | 1.89E-34 |
| IRG1 | −1.15 | 2.20E-34 |
| DOCK4 | −1.24 | 7.32E-34 |
| PDE3B | −1.17 | 2.36E-33 |
| PHIP | −1.15 | 4.33E-33 |
| RASA1 | −1.17 | 9.57E-31 |
| HERC5 | −1.22 | 1.00E-30 |
| ISG20 | −1.29 | 1.10E-28 |
| EPB41L2 | −1.12 | 1.33E-28 |
| USP18 | −1.04 | 1.87E-28 |
| C11orf31 | −1.17 | 9.22E-28 |
| CHID1 | 1.04 | 1.26E-27 |
| GBP1 | −1.01 | 3.53E-27 |
| TRIB3 | 1.5 | 4.13E-27 |
| FAF1 | −1.24 | 8.23E-27 |
| SMOX | 1.12 | 9.98E-27 |
| KREMEN1 | 1.05 | 3.98E-26 |
| DAPP1 | −1.33 | 5.17E-26 |
| KLHL21 | 1.19 | 5.17E-26 |
| GPR68 | 1.08 | 3.16E-25 |
| MTMR6 | −1.08 | 1.63E-24 |
| CD274 | −1.27 | 3.46E-24 |
| ITGB8 | −1.05 | 7.26E-24 |
| EXOC4 | −1.04 | 6.31E-23 |
| SMC2 | −1.26 | 9.32E-23 |
| TRIM22 | −1.02 | 1.16E-22 |
| TNC | −1.44 | 1.52E-22 |
| RB1 | −1.09 | 2.55E-22 |
| HAPLN3 | −2.1 | 2.62E-22 |
| USP53 | 1.09 | 1.22E-21 |
| MT2A | −1.27 | 3.08E-21 |
| IQCB1 | −1.33 | 3.20E-21 |
| CCL3L1 | −1.02 | 5.93E-21 |
| CCL3L3 | −1.02 | 6.16E-21 |
| MMP19 | 1.27 | 2.00E-20 |
| GFPT2 | −1.11 | 3.45E-20 |
| IRF8 | −1.04 | 2.17E-19 |
| HS2ST1 | −1.02 | 2.17E-19 |
| CDCP1 | 1.28 | 9.11E-19 |
| MYBPH | −1.18 | 1.02E-18 |
| UBE2E2 | −1.14 | 1.16E-18 |
| STC1 | 1.72 | 2.23E-18 |
| FCAR | 1.37 | 3.67E-18 |
| IFI44L | −2.61 | 3.84E-18 |
| ETV6 | −1.04 | 4.38E-18 |
| CBWD5 | −1.36 | 4.48E-18 |
| GPR176 | −1.49 | 4.66E-18 |
| CCDC14 | −1.11 | 5.26E-18 |
| FAM212B | 1.58 | 7.00E-18 |
| MMP3 | 2.99 | 8.54E-18 |
| S100A9 | 1.02 | 1.20E-17 |
| SEMA4C | 1.18 | 1.29E-17 |
| RAPH1 | 1.19 | 2.71E-17 |
| IL7 | −1.03 | 3.30E-17 |
| PAX5 | −1.38 | 1.13E-16 |
| ARID5A | −1.23 | 1.36E-16 |
| CD70 | −1.17 | 1.93E-16 |
| MLKL | −1.08 | 1.13E-15 |
| GBP1P1 | −1.14 | 3.76E-15 |
| NBPF12 | 1.05 | 7.40E-15 |
| ANK2 | −1.05 | 2.62E-14 |
| LIF | −1.04 | 3.42E-14 |
| KDM3A | 1.02 | 5.85E-14 |
| CCL1 | −1.5 | 9.52E-14 |
| UGGT2 | −1.05 | 1.52E-13 |
| LOC101927 | −1.12 | 1.54E-13 |
| CXCL5 | 2.6 | 1.79E-13 |
| SEMA4D | −1.09 | 2.46E-13 |
| PLA2G4A | −1.08 | 3.05E-13 |
| MET | 1.02 | 5.32E-13 |
| NRG2 | −1.07 | 1.03E-12 |
| NUAK2 | −1.09 | 4.49E-12 |
| ARAP2 | −1.36 | 4.97E-12 |
| RNVU1-19 | −2.01 | 1.45E-11 |
| CLDN23 | −1 | 2.99E-11 |
| IL24 | 1.3 | 3.09E-11 |
| PDE4A | 1.12 | 4.07E-11 |
| ATE1 | −1.02 | 1.72E-10 |
| CHSY3 | −1.68 | 3.91E-10 |
| SECTM1 | −1.53 | 4.13E-10 |
| CCL7 | −1.27 | 4.18E-10 |
| VEGFC | −1.02 | 8.15E-10 |
| NBPF20 | 1.1 | 1.43E-09 |
| GRIN2D | 1.1 | 1.64E-09 |
| CBWD3 | −1.81 | 1.65E-09 |
| PCDH9 | 1.01 | 4.11E-09 |
| FUK | −1.11 | 4.56E-09 |
| DRAXIN | 1.86 | 5.13E-09 |
| GCA | −1.1 | 6.70E-09 |
| KIFAP3 | −1.05 | 7.10E-09 |
| CDC42EP2 | 1.03 | 1.22E-08 |
| EHD2 | 1.1 | 1.73E-08 |
| UNC5C | −1.09 | 1.89E-08 |
| CH25H | −1.71 | 1.91E-08 |
| MAP2 | −1.73 | 2.48E-08 |
| ACRC | 1.19 | 4.19E-08 |
| MMP1 | 1.98 | 4.34E-08 |
| MMP10 | 2.79 | 4.35E-08 |
| DPYD-AS1 | −1.02 | 4.97E-08 |
| GCNT2 | −1.71 | 5.16E-08 |
| KLF4 | −1.16 | 7.03E-08 |
| STX1A | 1.08 | 9.60E-08 |
| KIAA1671 | −1.4 | 1.33E-07 |
| TNFAIP8L1 | 1.19 | 1.33E-07 |
| KDM6A | −1.2 | 1.34E-07 |
| PTPRO | −1.17 | 2.17E-07 |
| DCAF4L1 | 1.7 | 2.34E-07 |
| SLC44A5 | −1.37 | 2.41E-07 |
| KIF3A | 1.05 | 2.70E-07 |
| LOC102724 | −3.68 | 2.71E-07 |
| TTC29 | −1.1 | 2.96E-07 |
| MMP8 | 1.71 | 3.10E-07 |
| GTF2IRD1P | 1.11 | 3.12E-07 |
| VCAM1 | −1.7 | 3.21E-07 |
| MGAT4C | 1.3 | 4.04E-07 |
| RNU11 | −1.62 | 5.15E-07 |
| NRN1 | −1.34 | 6.74E-07 |
| PPBP | 1.87 | 7.64E-07 |
| TNFSF8 | −1.38 | 8.00E-07 |
| EMR3 | 1.67 | 8.76E-07 |
| HTRA3 | −1.42 | 9.43E-07 |
| PRR12 | 1.05 | 1.01E-06 |
| CBWD6 | −1.6 | 1.12E-06 |
| CFH | −1.41 | 1.13E-06 |
| ICAM4 | −1 | 1.69E-06 |
| ADRB2 | −1.02 | 1.70E-06 |
| C11orf96 | −1.39 | 1.81E-06 |
| PELI3 | 1.01 | 1.92E-06 |
| HESX1 | −1.36 | 2.17E-06 |
| CHI3L2 | −1.24 | 2.29E-06 |
| LOC100505 | −1.35 | 2.29E-06 |
| ARC | −1.1 | 2.77E-06 |
| IFITM1 | −1.44 | 2.99E-06 |
| RNU12 | −1.45 | 3.03E-06 |
| SHISA3 | 1.19 | 3.19E-06 |
| ANXA2R | −1.01 | 3.62E-06 |
| LOC103611 | 1.13 | 3.96E-06 |
| EPHB2 | −1.2 | 4.43E-06 |
| SORCS1 | −1.28 | 5.26E-06 |
| DPP4 | −1.28 | 6.56E-06 |
| FGD2 | −1.13 | 6.75E-06 |
| THY1 | −2.02 | 7.49E-06 |
| ZFP28 | 1.13 | 7.72E-06 |
| IFIT1B | −1.17 | 1.07E-05 |
| ABCA5 | −1.38 | 1.08E-05 |
| SMTNL1 | −1 | 1.09E-05 |
| WTAPP1 | 1.93 | 1.11E-05 |
| FAM26E | −1.08 | 1.14E-05 |
| NEXN | −1.17 | 1.28E-05 |
| KRBA1 | 1.13 | 1.33E-05 |
| CCR7 | −1.04 | 1.47E-05 |
| FRMD3 | −1.28 | 1.95E-05 |
| SRPX | −1.53 | 1.95E-05 |
| AREG | 1.33 | 2.11E-05 |
| CD1D | −1.14 | 2.13E-05 |
| SERPING1 | −1.31 | 2.33E-05 |
| PMFBP1 | −1.32 | 2.41E-05 |
| UTS2 | −1.09 | 2.74E-05 |
| CCNG2 | 1.29 | 3.90E-05 |
| ZNF558 | 1 | 4.66E-05 |
| TMPRSS13 | −1.42 | 5.83E-05 |
| FAM131A | 1.09 | 5.93E-05 |
| LOC101929 | 1.03 | 6.30E-05 |
| FGL2 | −1.24 | 7.39E-05 |
| ZNF786 | 1.14 | 7.50E-05 |
| OTOF | −1.75 | 1.02E-04 |
| LAMP3 | −1.04 | 1.11E-04 |
| THSD4 | −1.15 | 1.23E-04 |
| CACNA1I | −1.44 | 1.27E-04 |
| MMP12 | 1.3 | 1.29E-04 |
| LAMA5 | 1.07 | 1.34E-04 |
| SULT1C2 | −1.3 | 1.49E-04 |
| SOWAHC | 1.06 | 1.70E-04 |
| SLC22A17 | −1.24 | 1.71E-04 |
| SLC35F1 | −1.32 | 2.60E-04 |
| ZNF568 | 1.03 | 2.60E-04 |
| LOC100133 | 1.31 | 2.72E-04 |
| MIR4657 | 1.08 | 3.04E-04 |
| TUBA8 | −1.05 | 3.13E-04 |
| RNVU1-20 | −2.21 | 3.50E-04 |
| LOC101928 | −1.25 | 3.89E-04 |
| CHRM3 | −1.94 | 4.02E-04 |
| BANK1 | −1.42 | 4.21E-04 |
| MMP13 | −1.96 | 4.27E-04 |
| BTC | −1.3 | 4.34E-04 |
| MARCO | −1.01 | 4.41E-04 |
| ABTB1 | 1.19 | 4.52E-04 |
| MYPOP | 1.17 | 4.58E-04 |
| IFNB1 | −2 | 4.68E-04 |
| ATP2B2 | −1.07 | 5.12E-04 |
| TM4SF1 | 1.62 | 6.02E-04 |
| FCGR2B | −1.55 | 6.48E-04 |
| CALN1 | −1.07 | 6.75E-04 |
| CYP2J2 | −1.61 | 6.75E-04 |
| GALNT18 | −1.07 | 6.99E-04 |
| VGLL2 | −1.29 | 7.35E-04 |
| ZNF610 | 1.01 | 7.46E-04 |
| LINC00346 | −1.14 | 8.33E-04 |
| DAB2IP | −1.23 | 9.35E-04 |
| HSH2D | −1.03 | 9.62E-04 |
| MNDA | −1.4 | 1.05E-03 |
| ROS1 | −1.02 | 1.05E-03 |
| GIMAP4 | −1.37 | 1.07E-03 |
| LARGE | −1.27 | 1.15E-03 |
| CD101 | −1.44 | 1.32E-03 |
| PI15 | −1.09 | 1.35E-03 |
| EREG | 1.27 | 1.36E-03 |
| FAM46C | −1.54 | 1.56E-03 |
| SPSB4 | −1.5 | 1.60E-03 |
| PRKXP1 | 1.08 | 1.97E-03 |
| FXYD6-FX | −1.03 | 2.00E-03 |
| SALL1 | −1.06 | 2.08E-03 |
| ACTN2 | −1.33 | 2.27E-03 |
| SEP4 | −1.15 | 2.36E-03 |
| LILRB2 | −1.03 | 2.42E-03 |
| RASD1 | −1.43 | 2.43E-03 |
| RUFY4 | −1.36 | 2.63E-03 |
| PRTFDC1 | −1.46 | 2.79E-03 |
| AOC2 | 1.49 | 2.86E-03 |
| RNF128 | −1.37 | 3.31E-03 |
| KIF5C | −1.16 | 3.40E-03 |
| LILRA1 | −1.3 | 3.40E-03 |
| SCN3A | −1.86 | 3.42E-03 |
| ACTA2 | −2.31 | 3.53E-03 |
| ADAMTS14 | −1.21 | 3.76E-03 |
| FCRLB | −1.16 | 3.91E-03 |
| FAM47E | −1.48 | 4.13E-03 |
| GMPR | −1.04 | 4.20E-03 |
| MAMDC2 | −1.62 | 4.34E-03 |
| IL13RA2 | 1.5 | 4.44E-03 |
| LPPR4 | −1.86 | 4.44E-03 |
| C11orf94 | 1.86 | 4.59E-03 |
| FLJ39080 | −1.38 | 4.64E-03 |
| DYRK1B | 1.12 | 4.78E-03 |
| GRIK2 | −1.29 | 4.85E-03 |
| KCNH1 | −1.74 | 5.53E-03 |
| SCN5A | −1.32 | 5.55E-03 |
| OVGP1 | 1.13 | 5.56E-03 |
| IFI27 | −1.85 | 6.01E-03 |
| LYG1 | 1.87 | 6.30E-03 |
| MAP7D2 | 1.48 | 6.33E-03 |
| IL27 | −1.33 | 6.36E-03 |
| MIR6753 | 1.04 | 6.70E-03 |
| LINGO1 | −1.41 | 6.99E-03 |
| PRUNE2 | −1.28 | 7.31E-03 |
| CACNA1B | −1.24 | 7.43E-03 |
| SPARCL1 | −1.06 | 7.54E-03 |
| SUGCT | −1.8 | 7.62E-03 |
| IFNLR1 | −2 | 7.84E-03 |
| GLI3 | −1.53 | 7.88E-03 |
| GRIK3 | −1.55 | 8.42E-03 |
| BATF3 | −1.12 | 8.69E-03 |
| CRIPAK | 1.5 | 8.85E-03 |
| DLG2 | −1.58 | 9.09E-03 |
| FFAR2 | −1.2 | 9.19E-03 |
| RNVU1-8 | −1.6 | 9.83E-03 |
| ZNF77 | 1.07 | 1.02E-02 |
| SYT15 | −1.01 | 1.06E-02 |
| TTBK1 | −1.21 | 1.11E-02 |
| KCNIP2 | −1.02 | 1.17E-02 |
| FAXC | −1.78 | 1.23E-02 |
| CAMK2B | −1.04 | 1.24E-02 |
| DKFZP434I | 1.1 | 1.24E-02 |
| SELL | −2.01 | 1.30E-02 |
| LRP1B | −1.44 | 1.32E-02 |
| ZBP1 | −1.78 | 1.38E-02 |
| LOC101929 | −1.82 | 1.41E-02 |
| LOC101927 | −1.09 | 1.56E-02 |
| SNORD5 | 1.13 | 1.59E-02 |
| BACE2 | −1.51 | 1.60E-02 |
| FHOD3 | −1.11 | 1.63E-02 |
| BBOX1 | −1.32 | 1.65E-02 |
| HS6ST3 | −1.19 | 1.66E-02 |
| ZNF10 | 1 | 1.74E-02 |
| SERPIND1 | 1.49 | 1.80E-02 |
| ASTN1 | −1.47 | 1.80E-02 |
| CFB | −1.56 | 1.83E-02 |
| KCNA10 | −1.57 | 1.88E-02 |
| MBNL2 | −1.15 | 1.95E-02 |
| TMEM136 | −1.03 | 1.95E-02 |
| LOC100129 | 1.08 | 1.96E-02 |
| SLC26A3 | 1.33 | 1.96E-02 |
| ABCG4 | −1.46 | 1.97E-02 |
| PADI2 | −1.53 | 1.99E-02 |
| SLC6A17 | −1.46 | 2.02E-02 |
| NPIPB3 | 1.15 | 2.07E-02 |
| C16orf74 | −1.52 | 2.14E-02 |
| LEPREL2 | −1.24 | 2.21E-02 |
| SDK2 | −1.05 | 2.22E-02 |
| KCNIP1 | −1.42 | 2.24E-02 |
| ITK | −1.03 | 2.28E-02 |
| CSMD2 | −1.1 | 2.29E-02 |
| MOXD1 | −1.15 | 2.30E-02 |
| FAT3 | −1.41 | 2.31E-02 |
| FAM227A | 1.02 | 2.33E-02 |
| SND1-IT1 | 1.04 | 2.38E-02 |
| GPR31 | −1.69 | 2.50E-02 |
| CD300E | −1.15 | 2.51E-02 |
| SEMA3F | −1.33 | 2.60E-02 |
| ZNF300 | −1.66 | 2.64E-02 |
| SOCS2 | −1.38 | 2.66E-02 |
| IL20RB | 1.13 | 2.66E-02 |
| ADAMTSL1 | −1.1 | 2.70E-02 |
| CA8 | −1.56 | 2.74E-02 |
| LOC100506 | −1.21 | 2.75E-02 |
| SYT1 | −1.17 | 2.82E-02 |
| XDH | −1.3 | 2.89E-02 |
| ELAVL4 | −1.6 | 2.91E-02 |
| CHDH | −1.94 | 2.94E-02 |
| SYT17 | −1.27 | 2.96E-02 |
| AOC3 | 1.29 | 2.99E-02 |
| TDRD9 | 1.07 | 3.09E-02 |
| SEMA5A | −1.57 | 3.11E-02 |
| SAMD12 | −1.34 | 3.14E-02 |
| LEPREL4 | −1.01 | 3.33E-02 |
| GCNT7 | 1.11 | 3.33E-02 |
| CPXM1 | −1.02 | 3.38E-02 |
| NYNRIN | −1.5 | 3.41E-02 |
| LINC00607 | −1.3 | 3.47E-02 |
| TLE2 | −1.78 | 3.53E-02 |
| TRIM29 | −1.48 | 3.53E-02 |
| MAGI2 | −1.03 | 3.55E-02 |
| C1QC | −1.48 | 3.57E-02 |
| KLF2 | −1.26 | 3.58E-02 |
| PLSCR4 | −1.18 | 3.72E-02 |
| DRP2 | −1.39 | 3.73E-02 |
| CD34 | −1.09 | 3.82E-02 |
| HIST1H3E | −1.58 | 3.87E-02 |
| SIDT1 | −1.1 | 3.89E-02 |
| LILRA5 | −1.21 | 3.90E-02 |
| LOC152225 | 1.11 | 4.08E-02 |
| MAB21L3 | 1.08 | 4.16E-02 |
| BCL2L14 | −1.61 | 4.17E-02 |
| NGF | −1.66 | 4.18E-02 |
| CADM3 | −1.54 | 4.20E-02 |
| SLC24A2 | −1.46 | 4.26E-02 |
| PRKAG3 | −1.49 | 4.28E-02 |
| CYP2D7P | 1.27 | 4.30E-02 |
| NPM2 | −1.12 | 4.31E-02 |
| CDHR1 | −1.39 | 4.37E-02 |
| TRPC3 | −1.58 | 4.38E-02 |
| KIAA2022 | −1.09 | 4.40E-02 |
| SRGAP2-A | −1.16 | 4.57E-02 |
| FCRL5 | −1.57 | 4.59E-02 |
| MCF2L2 | −1.3 | 4.62E-02 |
| ZBED6CL | −1.13 | 4.65E-02 |
| HIST2H2BA | −1.06 | 4.87E-02 |
| LINC01366 | −1.62 | 4.94E-02 |
| ZNF709 | 1 | 4.95E-02 |
| CMKLR1 | −1.45 | 5.00E-02 |

Extended Data Table 5. Genes differentially expressed
in THP-1 macrophages overexpressing CHROMR versus empty
vector control by RT2 gene expression profiler

| Gene | Fold change (log2) | P-value (log10) |
|---|---|---|
| IFI27 | 5.54 | 1.7010 |
| IFITM1 | 3.98 | 1.1828 |
| MX1 | 2.89 | 1.5409 |
| MX2 | 2.51 | 1.5187 |
| CXCL10 | 2.36 | 1.9146 |
| IFIT1 | 2.18 | 1.0630 |
| OAS2 | 2.14 | 1.1278 |
| ISG15 | 2.11 | 1.4031 |
| MNDA | 2.10 | 1.5856 |
| TNFSF10 | 2.09 | 2.9614 |
| IFI6 | 1.98 | 1.0040 |
| OAS1 | 1.94 | 1.1123 |
| IFITM3 | 1.73 | 1.0246 |
| IRF7 | 1.63 | 0.8418 |
| DDX58 | 1.62 | 1.3135 |
| STAT1 | 1.54 | 1.0587 |
| BST2 | 1.44 | 1.3741 |
| TLR9 | 1.31 | 0.5360 |
| EIF2AK2 | 1.26 | 1.0214 |
| MYD88 | 1.24 | 1.0224 |
| IFI16 | 1.20 | 1.1422 |
| GBP1 | 1.20 | 1.0900 |
| IFIH1 | 1.04 | 0.6882 |
| IRF9 | 0.96 | 0.8561 |
| TIMP1 | 0.93 | 1.4447 |
| IFIT3 | 0.89 | 0.6743 |
| STAT2 | 0.84 | 0.6799 |
| ADAR | 0.70 | 0.5351 |
| TAP1 | 0.62 | 0.9380 |
| TLR3 | 0.59 | 0.4873 |
| PML | 0.54 | 0.4987 |
| IFITM2 | 0.52 | 0.4813 |
| PSME2 | 0.41 | 0.4247 |
| TICAM1 | 0.40 | 0.6056 |
| TYK2 | 0.39 | 0.4303 |
| HLA-A | 0.39 | 0.3388 |
| MAL | 0.32 | 0.4536 |
| IFI30 | 0.28 | 0.2763 |
| MET | 0.25 | 0.3608 |
| ISG20 | 0.25 | 0.2777 |
| VEGFA | 0.20 | 0.2554 |
| IFNW1 | 0.20 | 0.1928 |
| IRF3 | 0.19 | 0.2269 |
| TRAF3 | 0.19 | 0.2063 |
| IFNA1 | 0.18 | 0.1954 |
| CD80 | 0.18 | 0.1479 |
| CCL2 | 0.18 | 0.1029 |
| IFNA2 | 0.16 | 0.1802 |
| BAG3 | 0.16 | 0.0551 |
| HLA-E | 0.14 | 0.1458 |
| TMEM173 | 0.10 | 0.0462 |
| JAK2 | 0.06 | 0.0332 |
| HLA-G | 0.03 | 0.0039 |
| NMI | 0.00 | 0.0933 |
| STAT3 | 0.00 | 0.0723 |
| SHB | −0.03 | 0.0465 |
| CD86 | −0.04 | 0.1387 |
| CASP1 | −0.06 | 0.1303 |
| HLA-B | −0.06 | 0.0233 |
| CRP | −0.09 | 0.0923 |
| IFNA4 | −0.09 | 0.0923 |
| IFNE | −0.09 | 0.0923 |
| TLR8 | −0.09 | 0.0608 |
| IRF5 | −0.12 | 0.0439 |
| IL15 | −0.12 | 0.0232 |
| CCL5 | −0.14 | 0.1585 |
| CD70 | −0.15 | 0.2267 |
| IFNAR2 | −0.15 | 0.0289 |
| SOCS1 | −0.15 | 0.0213 |
| IRF2 | −0.15 | 0.0045 |
| IRF1 | −0.20 | 0.0399 |
| JAK1 | −0.25 | 0.1785 |
| CIITA | −0.27 | 0.1647 |
| IL6 | −0.27 | 0.0153 |
| CAV1 | −0.29 | 0.2659 |
| IFNAR1 | −0.32 | 0.0774 |
| CDKN1B | −0.34 | 0.3993 |
| SH2D1A | −0.45 | 0.2341 |
| TLR7 | −0.54 | 0.3492 |
| PRKCZ | −0.54 | 0.1866 |
| IFIT2 | −0.84 | 0.4784 |
| IFNB1 | −1.15 | 0.5381 |
| NOS2 | −1.43 | 1.2135 |
| IL10 | −3.06 | 0.9766 |

EXTENDED DATA TABLE 6
Table of oligonucleotides used in this study
qPCR

| Primer | Sequence |
|---|---|
| hGAPDH_F | GAAGGTGAAGGTCGGAGTC (SEQ ID NO: 119) |
| hGAPDH_R | GAAGATGGTGATGGGATTTC (SEQ ID NO: 120) |
| hACTB_F | TGCGTTACACCCTTTCTTGA (SEQ ID NO: 121) |
| hACTB_R | AAAGCCATGCCAATCTCATC (SEQ ID NO: 122) |
| hHPRT1_F | ACCCCACGAAGTGTTGGATA (SEQ ID NO: 123) |
| hHPRT1_R | AAGCAGATGGCCACAGAACT (SEQ ID NO: 124) |
| CHROMR-univ_F | GCAGGAGCTTGAATTTCAGT (SEQ ID NO: 125) |
| CHROMR-univ_R | TGTACTGAGTGGGCATTTAT (SEQ ID NO: 126) |
| CHROMR-1_F | GATAAAAGGCCATCATCAGACA (SEQ ID NO: 127) |
| CHROMR-1_R | CAAGCACAGGATTCCAAGGT (SEQ ID NO: 128) |
| CHROMR-3_F | CTGGAGTGCAGTGCTGTGAT (SEQ ID NO: 129) |
| CHROMR-3_R | ATCCCTTGAGGCCAAAAGTT (SEQ ID NO: 130) |
| CHROMR-4_F | TGCCTTCTGTCTTTTGTCCA (SEQ ID NO: 131) |
| CHROMR-4_R | GATTTGGGGCAAGTTGCTTA (SEQ ID NO: 132) |
| CHROMR-5_F | GCGTTAGCCAGAATGGTTTC (SEQ ID NO: 133) |
| CHROMR-5_R | GGACCACGTGAGGAAAAGTC (SEQ ID NO: 134) |
| CHROMR-6_F | ATGCTCATGCCTCAGTCTCC (SEQ ID NO: 135) |
| CHROMR-6_R | TGGAAAGCCATCTAAAAACCA (SEQ ID NO: 136) |
| CHROMR-7_F | ATACCGGAGCCATGACAGAC (SEQ ID NO: 137) |
| CHROMR-7_R | CGTCATCCTTGAATGTGTCATTA (SEQ ID NO: 138) |
| HOTAIR_F | GGTAGAAAAAGCAACCACGAAGC (SEQ ID NO: 139) |
| HOTAIR_R | ACATAAACCTCTGTCTGTGAGTGCC (SEQ ID NO: 140) |
| NEAT1_F | TCGGGTATGCTGTTGTGAAA (SEQ ID NO: 141) |
| NEAT1_R | TGACGTAACAGAATTAGTTCTTACCA (SEQ ID NO: 142) |

Mutation studies

| Primer | Sequence |
|---|---|
| CHROMR3_mut2_F | CTCAAATGGGGGACCTCCCAAAGCCCATATCCTGGCAGTG |
| | TG (SEQ ID NO: 143) |
| CHROMR3_mut2_R | CACACTGCCAGGATATGGGCTTTGGGAGGTCCCCCATTTG |
| | AG (SEQ ID NO: 144) |

ChIRP probes

| Probe | Sequence |
|---|---|
| #1 | TTTTGTGCCCATAGGTTCTT (SEQ ID NO: 145) |
| #2 | TCTTTTCTGGAAAATTCCTC (SEQ ID NO: 146) |
| #3 | CTTAAGGTGCCATCAAGTTC (SEQ ID NO: 147) |
| #4 | TTTCACACTGCCAGGATATC (SEQ ID NO: 148) |
| #5 | GGTAGGTCTCATGTATTCAT (SEQ ID NO: 149) |
| #6 | GCTAGCTTTCTACATTAGGT (SEQ ID NO: 150) |
| #7 | GTGACAGAGTGAGACCCTTT (SEQ ID NO: 151) |
| #8 | CTACTCAGGAGGCTGAGGTG (SEQ ID NO: 152) |
| #9 | AAAAGTTTGAGACCAGCCTG (SEQ ID NO: 153) |
| #10 | AAGCATTTGACCATGCATGT (SEQ ID NO: 154) |

1. A method comprising administering to an individual who has a disorder associated with type I interferon induced inflammation an agent that inhibits the function and/or reduces the level of long non-coding RNA Cholesterol Homeostasis Regulator of Micro-RNA expression RNA (lncRNA CHROMR) in the individual to thereby reduce the severity of the type I interferon induced inflammation.

2. The method of claim 1, wherein the agent comprises an antisense oligonucleotide targeted to the LncRNA CHROMR.

3. The method of claim 2, wherein the antisense oligonucleotide consists of 7-20 nucleotides.

4. The method of claim 3, wherein the antisense oligonucleotide comprises the sequence CCCCCAT or CTCATAAGAAAACTGA (SEQ ID NO: 1).

5. The method of claim 4, wherein the antisense oligonucleotide comprises the sequence GGACCTCCCCCAT (SEQ ID NO: 17).

6. The method of claim 1, wherein the type I interferon induced inflammation is associated with an autoimmune disorder and is optionally a type I inteferonopathy.

7. The method of claim 6, wherein the autoimmune disorder is Systemic lupus erythematosus (SLE), psoriasis, type I diabetes, rheumatoid arthritis, Sjögrens syndrome, dermatomyositis, Aicardi-Goutières Syndrome, Familial chilblain lupus, STING-associated vasculopathy spastic paraparesis, Singleton-Merten syndrome, Trichohepatoenteric syndrome, infantile encephalopathy, ataxia telangiectasia, Bloom syndrome, common variable immunodeficiency, proteasome-associated autoinflammatory syndrome.

8. The method of claim 6, wherein the type I interferon induced inflammation is associated with a pathology of the cardiovascular system of the individual.

9. The method of claim 8, wherein the pathology of the cardiovascular system comprises heart failure, wherein optionally the heart failure has been induced by treating the individual with interferon-β or is sepsis-induced cardiomyopathy

10. A method comprising selecting an individual that has been identified as disorder associated with type I interferon induced inflammation and administering to the individual an agent that inhibits the function and/or reduces the level of Cholesterol Homeostasis Regulator of Micro-RNA expression RNA (LncRNA CHROMR) in the individual.

11. The method of claim 10, wherein the agent comprises an antisense oligonucleotide targeted to the LncRNA CHROMR.

12. The method of claim 11, wherein the antisense oligonucleotide consists of 7-20 nucleotides.

13. The method of claim 12, wherein the antisense oligonucleotide comprises the sequence CCCCCAT.

14. The method of claim 13, wherein the antisense oligonucleotide comprises the sequence GGACCTCCCCCAT (SEQ ID NO: 17).

15. The method of claim 10, wherein the individual that has been identified as having disorder has been identified as having an autoimmune disorder.

16. The method of claim 15, wherein the autoimmune disorder is Systemic lupus erythematosus (SLE), type I diabetes, psoriasis, rheumatoid arthritis, Sjögrens syndrome, dermatomyositis, or multiple sclerosis.

17. The method of claim 15, wherein the disorder is associated with a pathology of the cardiovascular system of the individual.

18. The method of claim 17, wherein the pathology of the cardiovascular system comprises heart failure, wherein optionally the heart failure has been induced by treating the individual with interferon-beta.

19. An isolated oligonucleotide comprising the sequence CCCCCAT.

20. The isolated oligonucleotide of claim 19 comprising the sequence GGACCTCCCCCAT (SEQ ID NO: 17) or CTCATAAGAAAACTGA (SEQ ID NO: 1).

21. (canceled)


SEQUENCE LISTING (154 sequences)

SEQ ID NO: 1 - DNA, 16 bp, synthetic construct
  source 1..16: mol_type = other DNA
ctcataagaaaactga

SEQ ID NO: 2 - DNA, 13 bp, synthetic construct
  source 1..13: mol_type = other DNA
ggaggtcccccat

SEQ ID NO: 3 - DNA, 12 bp, synthetic construct
  source 1..12: mol_type = other DNA
cggtgactcagc

SEQ ID NO: 4 - DNA, 10 bp, synthetic construct
  source 1..10: mol_type = other DNA
aaaccgaaag

SEQ ID NO: 5 - DNA, 10 bp, synthetic construct
  source 1..10: mol_type = other DNA
acaacttccg

SEQ ID NO: 6 - DNA, 10 bp, synthetic construct
  source 1..10: mol_type = other DNA
ggtcagagtt

SEQ ID NO: 7 - DNA, 10 bp, synthetic construct
  source 1..10: mol_type = other DNA
ccggccggac

SEQ ID NO: 8 - DNA, 10 bp, synthetic construct
  source 1..10: mol_type = other DNA
tcgttccgcc

SEQ ID NO: 9 - DNA, 12 bp, synthetic construct
  source 1..12: mol_type = other DNA
cagacagaacac

SEQ ID NO: 10 - DNA, 12 bp, synthetic construct
  source 1..12: mol_type = other DNA
gcagtcggggac

SEQ ID NO: 11 - DNA, 13 bp, synthetic construct
  source 1..13: mol_type = other DNA
tgtaatcgtaaac

SEQ ID NO: 12 - DNA, 12 bp, synthetic construct
  source 1..12: mol_type = other DNA
gcggaaacgaaa

SEQ ID NO: 13 - DNA, 28 bp, synthetic construct
  source 1..28: mol_type = other DNA
gggggaggtcccaaagcggatatcctgg

SEQ ID NO: 14 - DNA, 28 bp, synthetic construct
  source 1..28: mol_type = other DNA
gggggacctcccaaagcccatatcctgg

SEQ ID NO: 15 - DNA, 30 bp, synthetic construct
  source 1..30: mol_type = other DNA
ggcacaaaaagcaggaacgagtagcggtgg

SEQ ID NO: 16 - DNA, 29 bp, synthetic construct
  source 1..29: mol_type = other DNA
ggctggtctcaaacttttggcctcaaggg

SEQ ID NO: 17 - DNA, 13 bp, synthetic construct
  source 1..13: mol_type = other DNA
ggacctcccccat

SEQ ID NO: 18 - DNA, 13 bp, synthetic construct
  source 1..13: mol_type = other DNA
tgccaggatatcc

SEQ ID NO: 19 - DNA, 13 bp, synthetic construct
  source 1..13: mol_type = other DNA
taatccgagccag

SEQ ID NO: 20 - DNA, 16 bp, synthetic construct
  source 1..16: mol_type = other DNA
gcggtcgggatagttt

SEQ ID NO: 21 - DNA, 10 bp, synthetic construct
  source 1..10: mol_type = other DNA
cctcccccat

SEQ ID NO: 22 - DNA, 10 bp, synthetic construct
  source 1..10: mol_type = other DNA
ctcccccatt

SEQ ID NO: 23 - DNA, 10 bp, synthetic construct
  source 1..10: mol_type = other DNA
tcccccattt

SEQ ID NO: 24 - DNA, 10 bp, synthetic construct
  source 1..10: mol_type = other DNA
cccccatttg

SEQ ID NO: 25 - DNA, 11 bp, synthetic construct
  source 1..11: mol_type = other DNA
acctcccccat

SEQ ID NO: 26 - DNA, 11 bp, synthetic construct
  source 1..11: mol_type = other DNA
cctcccccatt

SEQ ID NO: 27 - DNA, 11 bp, synthetic construct
  source 1..11: mol_type = other DNA
ctcccccattt

SEQ ID NO: 28 - DNA, 11 bp, synthetic construct
  source 1..11: mol_type = other DNA
tcccccatttg

SEQ ID NO: 29 - DNA, 11 bp, synthetic construct
  source 1..11: mol_type = other DNA
cccccatttga

SEQ ID NO: 30 - DNA, 12 bp, synthetic construct
  source 1..12: mol_type = other DNA
gacctcccccat

SEQ ID NO: 31 - DNA, 12 bp, synthetic construct
  source 1..12: mol_type = other DNA
acctcccccatt

SEQ ID NO: 32 - DNA, 12 bp, synthetic construct
  source 1..12: mol_type = other DNA
cctcccccattt

SEQ ID NO: 33 - DNA, 12 bp, synthetic construct
  source 1..12: mol_type = other DNA
ctcccccatttg

SEQ ID NO: 34 - DNA, 12 bp, synthetic construct
  source 1..12: mol_type = other DNA
tcccccatttga

SEQ ID NO: 35 - DNA, 12 bp, synthetic construct
  source 1..12: mol_type = other DNA
cccccatttgag

SEQ ID NO: 36 - DNA, 13 bp, synthetic construct
  source 1..13: mol_type = other DNA
gacctcccccatt

SEQ ID NO: 37 - DNA, 13 bp, synthetic construct
  source 1..13: mol_type = other DNA
acctcccccattt

SEQ ID NO: 38 - DNA, 13 bp, synthetic construct
  source 1..13: mol_type = other DNA
cctcccccatttg

SEQ ID NO: 39 - DNA, 13 bp, synthetic construct
  source 1..13: mol_type = other DNA
ctcccccatttga

SEQ ID NO: 40 - DNA, 13 bp, synthetic construct
  source 1..13: mol_type = other DNA
tcccccatttgag

SEQ ID NO: 41 - DNA, 13 bp, synthetic construct
  source 1..13: mol_type = other DNA
cccccatttgaga

SEQ ID NO: 42 - DNA, 14 bp, synthetic construct
  source 1..14: mol_type = other DNA
gggacctcccccat

SEQ ID NO: 43 - DNA, 14 bp, synthetic construct
  source 1..14: mol_type = other DNA
ggacctcccccatt

SEQ ID NO: 44 - DNA, 14 bp, synthetic construct
  source 1..14: mol_type = other DNA
gacctcccccattt

SEQ ID NO: 45 - DNA, 14 bp, synthetic construct
  source 1..14: mol_type = other DNA
acctcccccatttg

SEQ ID NO: 46 - DNA, 14 bp, synthetic construct
  source 1..14: mol_type = other DNA
cctcccccatttga

SEQ ID NO: 47 - DNA, 14 bp, synthetic construct
  source 1..14: mol_type = other DNA
ctcccccatttgag

SEQ ID NO: 48 - DNA, 14 bp, synthetic construct
  source 1..14: mol_type = other DNA
tcccccatttgaga

SEQ ID NO: 49 - DNA, 14 bp, synthetic construct
  source 1..14: mol_type = other DNA
cccccatttgagaa

SEQ ID NO: 50 - DNA, 15 bp, synthetic construct
  source 1..15: mol_type = other DNA
tgggacctcccccat

SEQ ID NO: 51 - DNA, 15 bp, synthetic construct
  source 1..15: mol_type = other DNA
gggacctcccccatt

SEQ ID NO: 52 - DNA, 15 bp, synthetic construct
  source 1..15: mol_type = other DNA
ggacctcccccattt

SEQ ID NO: 53 - DNA, 15 bp, synthetic construct
  source 1..15: mol_type = other DNA
gacctcccccatttg

SEQ ID NO: 54 - DNA, 15 bp, synthetic construct
  source 1..15: mol_type = other DNA
acctcccccatttga

SEQ ID NO: 55 - DNA, 15 bp, synthetic construct
  source 1..15: mol_type = other DNA
cctcccccatttgag

SEQ ID NO: 56 - DNA, 15 bp, synthetic construct
  source 1..15: mol_type = other DNA
ctcccccatttgaga

SEQ ID NO: 57 - DNA, 15 bp, synthetic construct
  source 1..15: mol_type = other DNA
tcccccatttgagaa

SEQ ID NO: 58 - DNA, 15 bp, synthetic construct
  source 1..15: mol_type = other DNA
cccccatttgagaag

SEQ ID NO: 59 - DNA, 16 bp, synthetic construct
  source 1..16: mol_type = other DNA
ttgggacctcccccat

SEQ ID NO: 60 - DNA, 16 bp, synthetic construct
  source 1..16: mol_type = other DNA
tgggacctcccccatt

SEQ ID NO: 61 - DNA, 16 bp, synthetic construct
  source 1..16: mol_type = other DNA
gggacctcccccattt

SEQ ID NO: 62 - DNA, 16 bp, synthetic construct
  source 1..16: mol_type = other DNA
ggacctcccccatttg

SEQ ID NO: 63 - DNA, 16 bp, synthetic construct
  source 1..16: mol_type = other DNA
gacctcccccatttga

SEQ ID NO: 64 - DNA, 16 bp, synthetic construct
  source 1..16: mol_type = other DNA
acctcccccatttgag

SEQ ID NO: 65 - DNA, 16 bp, synthetic construct
  source 1..16: mol_type = other DNA
cctcccccatttgaga

SEQ ID NO: 66 - DNA, 16 bp, synthetic construct
  source 1..16: mol_type = other DNA
ctcccccatttgagaa

SEQ ID NO: 67 - DNA, 16 bp, synthetic construct
  source 1..16: mol_type = other DNA
tcccccatttgagaag

SEQ ID NO: 68 - DNA, 16 bp, synthetic construct
  source 1..16: mol_type = other DNA
cccccatttgagaagg

SEQ ID NO: 69 - DNA, 17 bp, synthetic construct
  source 1..17: mol_type = other DNA
tttgggacctcccccat

SEQ ID NO: 70 - DNA, 17 bp, synthetic construct
  source 1..17: mol_type = other DNA
ttgggacctcccccatt

SEQ ID NO: 71 - DNA, 17 bp, synthetic construct
  source 1..17: mol_type = other DNA
tgggacctcccccattt

SEQ ID NO: 72 - DNA, 17 bp, synthetic construct
  source 1..17: mol_type = other DNA
gggacctcccccatttg

SEQ ID NO: 73 - DNA, 17 bp, synthetic construct
  source 1..17: mol_type = other DNA
ggacctcccccatttga

SEQ ID NO: 74 - DNA, 17 bp, synthetic construct
  source 1..17: mol_type = other DNA
gacctcccccatttgag

SEQ ID NO: 75 - DNA, 17 bp, synthetic construct
  source 1..17: mol_type = other DNA
acctcccccatttgaga

SEQ ID NO: 76 - DNA, 17 bp, synthetic construct
  source 1..17: mol_type = other DNA
cctcccccatttgagaa

SEQ ID NO: 77 - DNA, 17 bp, synthetic construct
  source 1..17: mol_type = other DNA
ctcccccatttgagaag

SEQ ID NO: 78 - DNA, 17 bp, synthetic construct
  source 1..17: mol_type = other DNA
tcccccatttgagaagg

SEQ ID NO: 79 - DNA, 17 bp, synthetic construct
  source 1..17: mol_type = other DNA
cccccatttgagaaggg

SEQ ID NO: 80 - DNA, 18 bp, synthetic construct
  source 1..18: mol_type = other DNA
ctttgggacctcccccat

SEQ ID NO: 81 - DNA, 18 bp, synthetic construct
  source 1..18: mol_type = other DNA
tttgggacctcccccatt

SEQ ID NO: 82 - DNA, 18 bp, synthetic construct
  source 1..18: mol_type = other DNA
ttgggacctcccccattt

SEQ ID NO: 83 - DNA, 18 bp, synthetic construct
  source 1..18: mol_type = other DNA
tgggacctcccccatttg

SEQ ID NO: 84 - DNA, 18 bp, synthetic construct
  source 1..18: mol_type = other DNA
gggacctcccccatttga

SEQ ID NO: 85 - DNA, 18 bp, synthetic construct
  source 1..18: mol_type = other DNA
ggacctcccccatttgag

SEQ ID NO: 86 - DNA, 18 bp, synthetic construct
  source 1..18: mol_type = other DNA
gacctcccccatttgaga

SEQ ID NO: 87 - DNA, 18 bp, synthetic construct
  source 1..18: mol_type = other DNA
acctcccccatttgagaa

SEQ ID NO: 88 - DNA, 18 bp, synthetic construct
  source 1..18: mol_type = other DNA
cctcccccatttgagaag

SEQ ID NO: 89 - DNA, 18 bp, synthetic construct
  source 1..18: mol_type = other DNA
ctcccccatttgagaagg

SEQ ID NO: 90 - DNA, 18 bp, synthetic construct
  source 1..18: mol_type = other DNA
tcccccatttgagaaggg

SEQ ID NO: 91 - DNA, 18 bp, synthetic construct
  source 1..18: mol_type = other DNA
cccccatttgagaagggc

SEQ ID NO: 92 - DNA, 19 bp, synthetic construct
  source 1..19: mol_type = other DNA
gctttgggacctcccccat

SEQ ID NO: 93 - DNA, 19 bp, synthetic construct
  source 1..19: mol_type = other DNA
ctttgggacctcccccatt

SEQ ID NO: 94 - DNA, 19 bp, synthetic construct
  source 1..19: mol_type = other DNA
tttgggacctcccccattt

SEQ ID NO: 95 - DNA, 19 bp, synthetic construct
  source 1..19: mol_type = other DNA
ttgggacctcccccatttg

SEQ ID NO: 96 - DNA, 19 bp, synthetic construct
  source 1..19: mol_type = other DNA
tgggacctcccccatttga

SEQ ID NO: 97 - DNA, 19 bp, synthetic construct
  source 1..19: mol_type = other DNA
gggacctcccccatttgag

SEQ ID NO: 98 - DNA, 19 bp, synthetic construct
  source 1..19: mol_type = other DNA
ggacctcccccatttgaga

SEQ ID NO: 99 - DNA, 19 bp, synthetic construct
  source 1..19: mol_type = other DNA
gacctcccccatttgagaa

SEQ ID NO: 100 - DNA, 19 bp, synthetic construct
  source 1..19: mol_type = other DNA
acctcccccatttgagaag

SEQ ID NO: 101 - DNA, 19 bp, synthetic construct
  source 1..19: mol_type = other DNA
cctcccccatttgagaagg

SEQ ID NO: 102 - DNA, 19 bp, synthetic construct
  source 1..19: mol_type = other DNA
ctcccccatttgagaaggg

SEQ ID NO: 103 - DNA, 19 bp, synthetic construct
  source 1..19: mol_type = other DNA
tcccccatttgagaagggc

SEQ ID NO: 104 - DNA, 19 bp, synthetic construct
  source 1..19: mol_type = other DNA
cccccatttgagaagggca

SEQ ID NO: 105 - DNA, 20 bp, synthetic construct
  source 1..20: mol_type = other DNA
cgctttgggacctcccccat

SEQ ID NO: 106 - DNA, 20 bp, synthetic construct
  source 1..20: mol_type = other DNA
gctttgggacctcccccatt

SEQ ID NO: 107 - DNA, 20 bp, synthetic construct
  source 1..20: mol_type = other DNA
ctttgggacctcccccattt

SEQ ID NO: 108 - DNA, 20 bp, synthetic construct
  source 1..20: mol_type = other DNA
tttgggacctcccccatttg

SEQ ID NO: 109 - DNA, 20 bp, synthetic construct
  source 1..20: mol_type = other DNA
ttgggacctcccccatttga

SEQ ID NO: 110 - DNA, 20 bp, synthetic construct
  source 1..20: mol_type = other DNA
tgggacctcccccatttgag

SEQ ID NO: 111 - DNA, 20 bp, synthetic construct
  source 1..20: mol_type = other DNA
gggacctcccccatttgaga

SEQ ID NO: 112 - DNA, 20 bp, synthetic construct
  source 1..20: mol_type = other DNA
ggacctcccccatttgagaa

SEQ ID NO: 113 - DNA, 20 bp, synthetic construct
  source 1..20: mol_type = other DNA
gacctcccccatttgagaag

SEQ ID NO: 114 - DNA, 20 bp, synthetic construct
  source 1..20: mol_type = other DNA
acctcccccatttgagaagg

SEQ ID NO: 115 - DNA, 20 bp, synthetic construct
  source 1..20: mol_type = other DNA
cctcccccatttgagaaggg

SEQ ID NO: 116 - DNA, 20 bp, synthetic construct
  source 1..20: mol_type = other DNA
ctcccccatttgagaagggc

SEQ ID NO: 117 - DNA, 20 bp, synthetic construct
  source 1..20: mol_type = other DNA
tcccccatttgagaagggca

SEQ ID NO: 118 - DNA, 20 bp, synthetic construct
  source 1..20: mol_type = other DNA
cccccatttgagaagggcat

SEQ ID NO: 119 - DNA, 19 bp, synthetic construct
  source 1..19: mol_type = other DNA
gaaggtgaaggtcggagtc

SEQ ID NO: 120 - DNA, 20 bp, synthetic construct
  source 1..20: mol_type = other DNA
gaagatggtgatgggatttc

SEQ ID NO: 121 - DNA, 20 bp, synthetic construct
  source 1..20: mol_type = other DNA
tgcgttacaccctttcttga

SEQ ID NO: 122 - DNA, 20 bp, synthetic construct
  source 1..20: mol_type = other DNA
aaagccatgccaatctcatc

SEQ ID NO: 123 - DNA, 20 bp, synthetic construct
  source 1..20: mol_type = other DNA
accccacgaagtgttggata

SEQ ID NO: 124 - DNA, 20 bp, synthetic construct
  source 1..20: mol_type = other DNA
aagcagatggccacagaact

SEQ ID NO: 125 - DNA, 20 bp, synthetic construct
  source 1..20: mol_type = other DNA
gcaggagcttgaatttcagt

SEQ ID NO: 126 - DNA, 20 bp, synthetic construct
  source 1..20: mol_type = other DNA
tgtactgagtgggcatttat

SEQ ID NO: 127 - DNA, 22 bp, synthetic construct
  source 1..22: mol_type = other DNA
gataaaaggccatcatcagaca

SEQ ID NO: 128 - DNA, 20 bp, synthetic construct
  source 1..20: mol_type = other DNA
caagcacaggattccaaggt

SEQ ID NO: 129 - DNA, 20 bp, synthetic construct
  source 1..20: mol_type = other DNA
ctggagtgcagtgctgtgat

SEQ ID NO: 130 - DNA, 20 bp, synthetic construct
  source 1..20: mol_type = other DNA
atcccttgaggccaaaagtt

SEQ ID NO: 131 - DNA, 20 bp, synthetic construct
  source 1..20: mol_type = other DNA
tgccttctgtcttttgtcca

SEQ ID NO: 132 - DNA, 20 bp, synthetic construct
  source 1..20: mol_type = other DNA
gatttggggcaagttgctta

SEQ ID NO: 133 - DNA, 20 bp, synthetic construct
  source 1..20: mol_type = other DNA
gcgttagccagaatggtttc

SEQ ID NO: 134 - DNA, 20 bp, synthetic construct
  source 1..20: mol_type = other DNA
ggaccacgtgaggaaaagtc

SEQ ID NO: 135 - DNA, 20 bp, synthetic construct
  source 1..20: mol_type = other DNA
atgctcatgcctcagtctcc

SEQ ID NO: 136 - DNA, 21 bp, synthetic construct
  source 1..21: mol_type = other DNA
tggaaagccatctaaaaacca

SEQ ID NO: 137 - DNA, 20 bp, synthetic construct
  source 1..20: mol_type = other DNA
ataccggagccatgacagac

SEQ ID NO: 138 - DNA, 23 bp, synthetic construct
  source 1..23: mol_type = other DNA
cgtcatccttgaatgtgtcatta

SEQ ID NO: 139 - DNA, 23 bp, synthetic construct
  source 1..23: mol_type = other DNA
ggtagaaaaagcaaccacgaagc

SEQ ID NO: 140 - DNA, 25 bp, synthetic construct
  source 1..25: mol_type = other DNA
acataaacctctgtctgtgagtgcc

SEQ ID NO: 141 - DNA, 20 bp, synthetic construct
  source 1..20: mol_type = other DNA
tcgggtatgctgttgtgaaa

SEQ ID NO: 142 - DNA, 26 bp, synthetic construct
  source 1..26: mol_type = other DNA
tgacgtaacagaattagttcttacca

SEQ ID NO: 143 - DNA, 42 bp, synthetic construct
  source 1..42: mol_type = other DNA
ctcaaatgggggacctcccaaagcccatatcctggcagtgtg

SEQ ID NO: 144 - DNA, 42 bp, synthetic construct
  source 1..42: mol_type = other DNA
cacactgccaggatatgggctttgggaggtcccccatttgag

SEQ ID NO: 145 - DNA, 20 bp, synthetic construct
  source 1..20: mol_type = other DNA
ttttgtgcccataggttctt

SEQ ID NO: 146 - DNA, 20 bp, synthetic construct
  source 1..20: mol_type = other DNA
tcttttctggaaaattcctc

SEQ ID NO: 147 - DNA, 20 bp, synthetic construct
  source 1..20: mol_type = other DNA
cttaaggtgccatcaagttc

SEQ ID NO: 148 - DNA, 20 bp, synthetic construct
  source 1..20: mol_type = other DNA
tttcacactgccaggatatc

SEQ ID NO: 149 - DNA, 20 bp, synthetic construct
  source 1..20: mol_type = other DNA
ggtaggtctcatgtattcat

SEQ ID NO: 150 - DNA, 20 bp, synthetic construct
  source 1..20: mol_type = other DNA
gctagctttctacattaggt

SEQ ID NO: 151 - DNA, 20 bp, synthetic construct
  source 1..20: mol_type = other DNA
gtgacagagtgagacccttt

SEQ ID NO: 152 - DNA, 20 bp, synthetic construct
  source 1..20: mol_type = other DNA
ctactcaggaggctgaggtg

SEQ ID NO: 153 - DNA, 20 bp, synthetic construct
  source 1..20: mol_type = other DNA
aaaagtttgagaccagcctg

SEQ ID NO: 154 - DNA, 20 bp, synthetic construct
  source 1..20: mol_type = other DNA
aagcatttgaccatgcatgt